SEQUENCE LISTING (12 sequences)

SEQ ID NO: 1 - protein, 3392 aa, Dengue virus type 1
  MISC_FEATURE (2494)..(3392): Nonstructural protein 5 (NS5) in DENV1 Westpac74
MNNQRKKTGRPSFNMLKRARNRVSTVSQLAKRFSKGLLSGQGPMKLVMAFIAFLRFLAIPPTAGILARWGSFKKNGAIKVLRGFKKEISNMLNIMNRRKRSVTMLLMLLPTALAFHLTTRGGEPHMIVSKQERGKSLLFKTSAGVNMCTLIAMDLGELCEDTMTYKCPRITETEPDDVDCWCNATETWVTYGTCSQTGEHRRDKRSVALAPHVGLGLETRTETWMSSEGAWKQIQKVETWALRHPGFTVIALFLAHAIGTSITQKGIIFILLMLVTPSMAMRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKTIHEWCCRSCTLPPLRFKGEDGCWYGMEIRPVKEKEENLVKSMVSAGSGEVDSFSLGLLCISIMIEEVMRSRWSRKMLMTGTLAVFLLLTMGQLTWNDLIRLCIMVGANASDKMGMGTTYLALMATFRMRPMFAVGLLFRRLTSREVLLLTVGLSLVASVELPNSLEELGDGLAMGIMMLKLLTDFQSHQLWATLLSLTFVKTTFSLHYAWKTMAMILSIVSLFPLCLSTTSQKTTWLPVLLGSLGCKPLTMFLITENKIWGRKSWPLNEGIMAVGIVSILLSSLLKNDVPLAGPLIAGGMLIACYVISGSSADLSLEKAAEVSWEEEAEHSGASHNILVEVQDDGTMKIKDEERDDTLTILLKATLLAISGVYPMSIPATLFVWYFWQKKKQRSGVLWDTPSPPEVERAVLDDGIYRILQRGLLGRSQVGVGVFQEGVFHTMWHVTRGAVLMYQGKRLEPSWASVKKDLISYGGGWRFQGSWNAGEEVQVIAVEPGKNPKNVQTAPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIKRKLRTLVLAPTRVVASEMAEALKGMPIRYQTTAVKSEHTGKEIVDLMCHATFTMRLLSPVRVPNYNMIIMDEAHFTDPASIAARGYISTRVGMGEAAAIFMTATPPGSVEAFPQSNAVIQDEERDIPERSWNSGYDWITDFPGKTVWFVPSIKSGNDIANCLRKNGKRVVQLSRKTFDTEYQKTKNNDWDYVVTTDISEMGANFRADRVIDPRRCLKPVILKDGPERVILAGPMPVTVASAAQRRGRIGRNQNKEGDQYIYMGQPLKNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEIWTKEGERKKLRPRWLDARTYSDPLALREFKEFAAGRRSVSGDLILEIGKLPQHLTQRAQNALDNLVMLHNSEQGGKAYRHAMEELPDTIETLMLLALIAVLTGGVTLFFLSGRGLGKTSIGLLCVIASSALLWMASVEPHWIAASIILEFFLMVLLIPEPDRQRTPQDNQLAYVVIGLLFMILTVAANEMGLLETTKKDLGIGHAAAENHHHAAMLDVDLHPASAWTLYAVATTIITPMMRHTIENTTANISLTAIANQAAILMGLDKGWPISKMDIGVPLLALGCYSQVNPLTLTAAVFMLVAHYAIIGPGLQAKATREAQKRTAAGIMKNPTVDGIVAIDLDPVVYDAKFEKQLGQIMLLILCTSQILLMRTTWALCESITLATGPLTTLWEGSPGKFWNTTIAVSMANIFRGSYLAGAGLAFSLMKSLGGGRRGTGAQGETLGEKWKRQLNQLSKSEFNTYKRSGIIEVDRSEAKEGLKRGETTKHAVSRGTAKLRWFVERNLVKPEGKVIDLGCGRGGWSYYCAGLKKVTEVKGYTKGGPGHEEPIPMATYGWNLVKLYSGKDVFFTPPEKCDTLLCDIGESSPNPTIEEGRTLRVLKMVEPWLRGNQFCIKILNPYMPSVVETLEQMQRKHGGMLVRNPLSRNSTHEMYWVSCGTGNIVSAVNMTSRMLLNRFTMAHRKPTYERDVDLGAGTRHVAVEPEVANLDIIGQRIENIKNEHKSTWHYDEDNPYKTWAYHGSYEVKPSGSASSMVNGVVRLLTKPWDVIPMVTQIAMTDTTPFGQQRVFKEKVDTRTPKAKRGTAQIMEVTARWLWGFLSRNKKPRICTREEFTRKVRSNAAIGAVFVDENQWNSAKEAVEDERFWDLVHRERELHKQGKCATCVYNMMGKREKKLGEFGKAKGSRAIWYMWLGARFLEFEALGFMNEDHWFSRENSLSGVEGEGLHKLGYILRDISKIPGGNMYADDTAGWDTRITEDDLQNEAKITDIMEPEHALLATSIFKLTYQNKVVRVQRPAKNGTVMDVISRRDQRGSGQVGTYGLNTFTNMEAQLIRQMESEGIFSPSELETPNLAERVLDWLKKHGTERLKRMAISGDDCVVKPIDDRFATALTALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEDVPYLGKREDQWCGSLIGLTARATWATNIQVAINQVRRLIGNENYLDFMTSMKRFKNESDPEGALW

SEQ ID NO: 2 - protein, 3391 aa, Dengue virus type 2
  MISC_FEATURE (2492)..(3391): Non-structural protein 5 (NS5) in DENV-2 TSV01 wildtype
MNNQRKKARNTPFNMLKRERNRVSTVQQLTKRFSLGMLQGRGPLKLFMALVAFLRFLTIPPTAGILKRWGTIKKSKAINVLRGFRKEIGRMLNILNRRRRTAGIIIMMIPTVMAFHLTTRNGEPHMIVSRQEKGKSLLFKTENGVNMCTLMAMDLGELCEDTITYNCPLLRQNEPEDIDCWCNSTSTWVTYGTCTATGEHRREKRSVALVPHVGMGLETRTETWMSSEGAWKHAQRIETWVLRHPGFTIMAAILAYTIGTTYFQRVLIFILLTAVTPSMTMRCIGISNRDFVEGVSGGSWVDIVLEHGSCVTTMAKNKPTLDFELVKTEAKHPATLRKYCIEAKLTNTTTASRCPTQGEPSLNEEQDKRFVCKHSMVDRGWGNGCGLFGKGGIVTCAMFTCKKNMEGKVVQPENLEYTIVITPHSGEENAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLSWFKKGSSIGQMFETTMRGAKRMAILGDTAWDFGSLGGVFTSIGKALHQVFGAIYGAAFSGVSWTMKILIGVVITWIGMNSRSTSLSVSLVLVGVVTLYLGVMVQADSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKAWGKAKMLSTELHNHTFLIDGPETAECPNTNRAWNSLEVEDYGFGVFTTNIWLKLKERQDVFCDSKLMSAAIKDNRAVHADMGYWIESALNDTWKIEKASFIEVKSCHWPKSHTLWSNGVLESEMIIPKNFAGPVSQHNYRPGYHTQTAGPWHLGRLEMDFDFCEGTTVVVTEDCGNRGPSLRTTTASGKLITEWCCRSCTLPPLRYRGEDGCWYGMEIRPLKEKEENLVNSLVTAGHGQIDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKIVRNMEKYQLAVTIMAILCVPNAVILQNAWKVSCTTLAVVSVSPLLLTSSQQKADWIPLALTIKGLNPTAIFLTTLSRTSKKRSWPLNEAIMAVGMVSILASSLLKNDIPMTGPLVAGGLLTVCYVLTGRSADLELERAADVRWEEQAEISGSSPILSITISEDGSMSIKNEEEEQTLTILIRTGLLVISGLFPASIPITAAAWYLWEVKKQRAGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNTGTIGAVSLDFSPGTSGSPIVDKKGKVVGLYGNGVVTRSGAYVSAIAQTEKSIEDNPEIEDDIFRKKRLTIMDLHPGAGKTKRYLPAIVREAIKRGLRTLILAPTRVVAAEMEEALRGLPIRYQTPAIRAEHTGREIVDLMCHATFTMRLLSPIRVPNYNLIIMDEAHFTDPASIAARGYISTRVEMGEAAGIFMTATPPGSRDPFPQSNAPIMDEEREIPERSWNSGHEWVTDFKGKTVWFVPSIKAGNDIAACLRKNGKKVIQLSRKTFDSEYIKTRTNDWDFVVTTDISEMGANFKAERVIDPRRCMKPVILTDGEERVILAGPMPVTHSSAAQRRGRVGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARNALDNLAVLHTAEAGGRAYNHALSELPETLETLLLLTLLATVTGGIFLFLMSGKGIGKMTLGMCCIITASILLWYAQIQPHWIAASIILEFFLIVLLIPEPEKQRTPQDNQLTYVVIAILTVVAATMANEMGFLEKTKKDFGLGSIATQQPESNILDIDLRPASAWTLYAVATTFITPMLRHSIENSSVNVSLTAIANQATVLMGLGKGWPLSKMDIGVPLLAIGCYSQVNPITLTAALLLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTANTRRGTGNTGETLGEKWKNRLNALGKSEFQIYKKSGIQEVDRTLAKEGIKRGETDHHAVSRGSAKLRWFVERNLVTPEGKVVDLGCGRGGWSYYCGGLKNVKEVKGLTKGGPGHEEPIPMSTYGWNLVRLQSGVDVFFTPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRHKKATYEPDVDLGSGTRNIGIESETPNLDIIGKRIEKIKQEHETSWHYDQDHPYKTWAYHGSYETKQTGSASSMVNGVVRLLTKPWDIIPMVTQMAMTDTTPFGQQRVFKEKVDTRTQEPKEGTKKLMKITAEWLWKELGKKKTPRMCTREEFTRKVRSNAALGAIFTDENKWKSAREAVEDSGFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVVTYGLNTFTNMEAQLIRQMEGEGVFKSIQHLTVTEEIAVKNWLVRVGRERLSRMAISGDDCVVKPLDDRFASALTALNDMGKVRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHATHEWMTTEDMLTVWNRVWIQENPWMEDKTPVESWEEIPYLGKREDQWCGSLIGLTSRATWAKNIQTAINQVRSLIGNEEYTDYMPSMKRFRREEEEAGVLW

SEQ ID NO: 3 - protein, 3390 aa, Dengue virus type 3
  MISC_FEATURE (2491)..(3390): Non-structural protein 5 in DENV-3 D3MY05-34640 Wildtype
MNNQRKKTGKPSINMLKRVRNRVSTGSQLAKRFSRGLLNGQGPMKLVMAFIAFLRFLAIPPTAGILARWGTFKKSGAIKVLRGFKKEISNMLSIINRRKKTSLCLMMMLPATLAFHLTSRDGEPRMIVGKNERGKSLLFKTASGINMCTLIAMDLGEMCDDTVTYKCPLITEVEPEDIDCWCNLTSTWVTYGTCNQAGEHRRDKRSVALAPHVGMGLDTRAQTWMSAEGAWRQVEKVETWAFRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNVTTDSRCPTQGEAILPEEQDQNYVCKHTYVDRGWGNGCGLFGKGSLVTCAKFQCLELIEGKVVQHENLKYTVIITVHTGDQHQVGNETQGVTAEITPQASTVEAILPEYGTLGLECSPRTGLDFNEMILLTMKNKAWMVHRQWFFDLPLPWTSGATTETPTWNKKELLVTFKNAHAKKQEVVVLGSQEGAMHTALTGATEIQTSGGTSIFAGHLKCRLKMDKLELKGMSYAMCSNAFVLKKEVSETQHGTILIKVEYKGEDAPCKIPFSTEDGQGKAHNGRLITANPVVTKKEEPVNIEAEPPFGESNIIIGTGDKALKINWYKKGSSIGKMFEATARGARRMAILGDTAWDFGSVGGVLNSLGKMVHQIFGSAYTALFSGVSWIMKIGIGVLLTWIGLNSKNTSMSFSCIVIGIITLYLGAVVQADMGCVINWKGKELKCGSGIFVTNEVHTWTEQYKFQADSPKRLATAIAGAWENGVCGIRSTTRMENLLWKQIANELNYILWENNIKLTVVVGDIIGILEQGKRTLTPQPMELKYSWKTWGKAKIVTAEIQNSSFIIDGPNTPECPNASRAWNVWEVEDYGFGVFTTNIWLKLREMYTQLCDHRLMSAAVKDERAVHADMGYWIESQKNGSWKLEKASLIEVKTCTWPKSHTLWSNGVLESDMIIPKSLAGPISQHNYRPGYHTQTAGPWHLGKLELDFNYCEGTTVVITENCGTRGPSLRTTTVSGKLIHEWCCRSCTLPPLRYMGEDGCWYGMEIRPINEKEENMVKSLVSAGSGKVDNFTMGVLCLAILFEEVMRGKFGKKHMIAGVLFTFVLLLSGQITWRDMARTLIMIGSNASDRMGMGVTYLALIATFKIQPFLALGFFLRKLTSRENLLLGVGLAMATTLQLPEDIEQMANGIALGLMALKLITQFETYQLWTALVSLMCSNTIFTLTVAWRTATLILAGISLLPVCQSSSMRKTDWLPMTVAAMGVPPLPLFIFSLKDTLKRRSWPLNEGVMAVGLVSILASSLLRNDVPMAGPLVAGGLLIACYVITGTSADLTVEKAADVTWEEEAEQTGVSHNLMITVDDDGTMRIKDDETENILTVLLKTALLIVSGIFPYSIPATLLVWHTWQKQTQRSGVLWDVPSPPETQKAELEEGVYRIKQQGIFGKTQVGVGVQKEGVFHTMWHVTRGAVLTYNGKRLEPNWASVKKDLISYGGGWRLSAQWQKGEEVQVIAVEPGKNPKNFQTMPGIFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGKEIVDLMCHATFTMRLLSPVRVPNYNLIIMDEAHFTDPASIAARGYISTRVGMGEAAAIFMTATPPGTADAFPQSNAPIQDEERDIPERSWNSGNDWITDFAGKTVWFVPSIKAGNDIANCLRKNGKKVIQLSRKTFDTEYQKTKLNDWDFVVTTDISEMGANFKADRVIDPRRCLKPVILTDGPERVILAGPMPVTVASAAQRRGRVGRNPQKENDQYIFTGQPLNNDEDHAHWTEAKMLLDNINTPEGIIPALFEPEREKSAAIDGEYRLKGESRKTFVELMRRGDLPVWLAHKVASEGIKYTDRKWCFDGERNNQILEENMDVEIWTKEGEKKKLRPRWLDARTYSDPLALKEFKDFAAGRKSIALDLVTEIGRVPSHLAHRTRNALDNLVMLHTSEHGGRAYRHAVEELPETMETLLLLGLMILLTGGAMLFLISGKGVGKTSIGLICVVASSGMLWMADIPLQWIASAIVLEFFMMVLLIPEPEKQRTPQDNQLAYVVIGILTLAAIVAANEMGLLETTKRDLGMSKEPGVASPTSYLDVDLHPASAWTLYAVATTVITPMLRHTIENSTANVSLAAIANQAVVLMGLDKGWPISKMDLGVPLLALGCYSQVNPLTLTAAVLLLVTHYAIIGPGLQAKATREAQKRTAAGIMKNPTVDGIMTIDLDPVIYDSKFEKQLGQVMLLVLCAVQLLLMRTSWAFCEALTLATGPITTLWEGSPGKFWNTTIAVSMANIFRGSYLAGAGLAFSIMKSVGTGKRGTGSQGETLGEKWKKKLNQLSWKEFDLYKKSGITEVDRIEAKEGLKRGEITHHAVSRGSAKLQWFVERNMVIPEGRVIDLGCGRGGWSYYCAGLKKVTEVRGYTKGGPGHEEPVPMSTYGWNIVKLMSGKDVFYLPPEKCDTLLCDIGESSPSPTVEESRTIRVLKMVEPWLKNNQFCIKVLNPYMPAVIEHLERLQRKHGGMLVRNPLSRNSTHEMYWISNGTGNIVSSVNMVSRLLLNRFTMTYRKPTIEKDVDLGAGTRHVNAEPETPNMDVIGERIRRIKEEHSSTWHYDDENPYKTWAYHGSYEVKATGSASSMINGVVKLLTKPWDVVPTVTQMAMTDTTPFGQQRVFKEKVDTRTPKPMPGTRKVMEITAEWLWRTLGRNKRPRLCTREEFTKKVRTNAAMGAVFTEENQWDSARAAVEDEEFWKLVDRERELHKLGKCGSCVYNMMGKREKKLGEFGKAKGSRAIWYMWLGARYLEFEALGFLNEDHWFSRENSYSGVEGEGLHKLGYILRDISKIPGGAMYADDTAGWDTRITEDDLHNEEKITQQMDPEHRQLANAIFKLTYQNKVVKVQRPTPKGTVMDIISRKDQRGSGQVGTYGLNTFTNMEAQLIRQMEGEGVLSKTDLENPHLLEKKITQWLETKGVERLKRMAISGDDCVVKPIDDRFANALLALNDMGKVRKDIPQWQPSKGWQDWQQVPFCSHHFHELIMKDGRKLVVPCRPQDELIGRARISQGAGWSLKETACLGKAYAQMWALMYFHRRDLRLASNAICSAVPVHWVPTSRTTWSIHAHHQWMTTEDMLTVWNRVWIEDNPWMEDKTPVTTWEDVPYLGKREDQWCGSLIGLTSRATWAQNILTAIQQVRSLIGNEEFLDYMPSMKRFRKEEESEGAIW

SEQ ID NO: 4 - protein, 3387 aa, Dengue virus type 4
  MISC_FEATURE (2488)..(3387): Non-structural protein 5 (NS5) in DENV-4 D4MY01-22713 Wildtype
MNQRKKVVRPPFNMLKRERNRVSTPQGLVKRFSTGLFSGKGPLRMVLAFITFLRVLSIPPTAGILKRWGQLKKNKAIKILIGFRKEIGRMLNILNRRRRSTMTLLCLIPTVMAFHLSTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYGMRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTAVVKVKYEGAGAPCKIPIEIRDVNKEKVVGRIISSTPFAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWIIRILIGLLVLWIGTNSRNTSMAMTCIAVGGITLFLGFTVQADMGCVVSWNGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVNDLKYSWKTWGKAKIFTPEARNSTFLIDGPDTSECPNERRAWNFFEVEDYGFGMFTTNIWMKFREGSSEVCDHRLMSAAIKDQKAVHADMGYWIESSKNQTWQIEKASLIEVKTCLWPKTHTLWSNGVLESQMLIPRSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTAGQGTSETFSMGLLCLTLFVEECLRRRVTRKHMILVVVITFCAIILGGLTWMDLLRALIMLGDTMSGRIGGQIHLAIMAVFKMSPGYVLGVFLRKLTSRETALMVIGMAMTTVFSIPHDLMELIDGISLGLILLKIVTHFDNTQVGTLALSLTFIRSTTPLVMAWRTIMAVFFVVTLIPLCRTSCLQKQSHWVEITALILGAQALPVYLMTLMKGASRRSWPLNEGIMAVGLVSLLGSALLKNDVPLAGPMVAGGLLLAAYVMSGSSADLSLEKAANVQWDEMADITGSSPIIEVKQDEDGSFSIRDVEETNMITLLVKLALITVSGLYPLAIPVTMALWYIWQVKTQRSGALWDVPSPAATQKATLSEGVYRIMQRGLFGKTQVGVGIHMEGVFHTMWHVTRGSVICHETGRLEPSWADVRNDMISYGGGWRLGDKWDKEEDVQVLAIEPGKNPKHVQTKPGLFKTLTGEIGAVTLDFKPGTSGSPIINKKGKVIGLYGNGVVTKSGDYVSAITQAERIGEPDYEVDEDIFRKKRLTIMDLHPGAGKTKRILPSIVREALKRRLRTLILAPTRVVAAEMEEALRGLPIRYQTPAVKSDHTGREIVDLMCHATFTTRLLSSTRVPNYNLIVMDEAHFTDPCSVAARGYISTRVEMGEAAAIFMTATPPGSIDPFPQSNSPIEDIEREIPERSWNTGFDWITDYQGKTVWFVPSIKAGNDIANCLRKSGKRVIQLSRKTFDTEYPKTKLTDWDFVVTTDISEMGANFRAGRVIDPRRCLKPVILTDGPERVILAGPIPVTPASAAQRRGRIGRNPAQEDDQYVFSGDPLKNDEDHAHWTEAKMLLDNIYTPEGIIPTLFGPEREKTQAIDGEFRLRGEQRKTFVELMRRGDLPVWLSYKVASAGISYKDREWCFTGERNNQILEENMEVEIWTREGEKKKLRPKWLDARVYADPMALKDFKEFASGRKSITLDILTEIATLPTYLSSKAKLALDNIVMLHTTEKGGRAYQHALNELPESLETLMLVALLGAMTAGIFLFFMQGKGIGKLSMGLIAIAVASGLLWVAEIQPQWIAASIILEFFLMVLLIPEPEKQRTPQDNQLIYVILTILTIIGLIAANEMGLIEKTKTDFGFYQVKTETTILDVDLRPASAWTLYAVATTILTPMLRHTIENTSANLSLAAIANQAAVLMGLGKGWPLHRMDLGVPLLAMGCYSQVNPTTLTASLVMLLVHYAIIGPGLQAKATREAQKRTAAGIMKNPTVDGITVIDLEPISYDPKFEKQLGQVMLLVLCVGQLLLMRTTWALCEVLTLATGPIMTLWEGNPGRFWNTTIAVSTANIFRGSYLAGAGLAFSLIKNVQTPRRGTGTTGETLGEKWKRQLNSLDRKEFEEYKRSGILEVDRTEAKSALRDGSKIKHAVSRGSSKIRWIVERGMIKPKGKVVDLGCGRGGWSYYMATLKNVTEVKGYTKGGPGHEEPIPMATYGWNLVKLHSGVDVFYKPTEQVDTLLCDIGESSSNPTIEEGRTLRVLKMVEPWLSSKPEFCIKVLNPYMPTVIEELEKLQRRHGGSLVRCPLSRNSTHEMYWVSGASGNIVSSVNTISKMLLNRFTTRHRKPTYEKDVDLGAGTRSVSTETEKPDMTIIGRRLQRLREEHKETWHYDQENPYRTWAYHGSYEAPSTGSASSMVNGVVKLLTKPWDVIPMVTQLAMTDTTPFGQQRVFKEKVDTRTPQPKPGTRMIMTTTANWLWALLGKKKNPRLCTREEFISKVRSNAAIGAVFQEEQGWTSASEAVNDSRFWELVDKERALHQEGKCESCVYNMMGKREKKLGEFGRAKGSRAIWYMWLGARFLEFEALGFLNEDHWFSRENSWSGVEGEGLHRLGYILEDIDKKDGDLIYADDTAGWDTRITEDDLLNEELITEQMAPHHKTLAKAIFKLTYQNKVVKVLRPTPKGAVMDIISRKDQRGSGQVGTYGLNTFTNMEVQLIRQMEAEGVITQDDMQNPKGLKERVEKWLKECGVDRLKRMAISGDDCVVKPLDERFSTSLLFLNDMGKVRKDIPQWEPSKGWKNWQEVPFCSHHFHKIFMKDGRSLVVPCRNQDELIGRARISQGAGWSLRETACLGKAYAQMWSLMYFHRRDLRLASMAICSAVPTEWFPTSRTTWSIHAHHQWMTTEDMLKVWNRVWIEDNPNMTDKTPVHSWEDIPYLGKREDLWCGSLIGLSSRATWAKNIHTAITQVRNLIGKEEYVDYMPVMKRYSAPSESEGVL

SEQ ID NO: 5 - DNA, 10735 bp, Dengue virus type 1
  misc_feature (7574)..(10270): Non-structural protein 5 sequence in Dengue virus type 1 clone WestPac, cDNA of complete RNA genome (gi 1854036; gb U88535.1; DVU88535)
agttgttagtctacgtggaccgacaagaacagtttcgaatcggaagcttgcttaacgtagttctaacagttttttattagagagcagatctctgatgaacaaccaacggaaaaagacgggtcgaccgtctttcaatatgctgaaacgcgcgagaaaccgcgtgtcaactgtttcacagttggcgaagagattctcaaaaggattgctttcaggccaaggacccatgaaattggtgatggcttttatagcattcctaagatttctagccatacctccaacagcaggaattttggctagatggggctcattcaagaagaatggagcgatcaaagtgttacggggtttcaagaaagaaatctcaaacatgttgaacataatgaacaggaggaaaagatctgtgaccatgctcctcatgctgctgcccacagccctggcgttccatctgaccacccgagggggagagccgcacatgatagttagcaagcaggaaagaggaaaatcacttttgtttaagacctctgcaggtgtcaacatgtgcacccttattgcaatggatttgggagagttatgtgaggacacaatgacctacaaatgcccccggatcactgagacggaaccagatgacgttgactgttggtgcaatgccacggagacatgggtgacctatggaacatgttctcaaactggtgaacaccgacgagacaaacgttccgtcgcactggcaccacacgtagggcttggtctagaaacaagaaccgaaacgtggatgtcctctgaaggcgcttggaaacaaatacaaaaagtggagacctgggctctgagacacccaggattcacggtgatagccctttttctagcacatgccataggaacatccatcacccagaaagggatcatttttattttgctgatgctggtaactccatccatggccatgcggtgcgtgggaataggcaacagagacttcgtggaaggactgtcaggagctacgtgggtggatgtggtactggagcatggaagttgcgtcactaccatggcaaaagacaaaccaacactggacattgaactcttgaagacggaggtcacaaaccctgccgtcctgcgcaaactgtgcattgaagctaaaatatcaaacaccaccaccgattcgagatgtccaacacaaggagaagccacgctggtggaagaacaggacacgaactttgtgtgtcgacgaacgttcgtggacagaggctggggcaatggttgtgggctattcggaaaaggtagcttaataacgtgtgctaagtttaagtgtgtgacaaaactggaaggaaagatagtccaatatgaaaacttaaaatattcagtgatagtcaccgtacacactggagaccagcaccaagttggaaatgagaccacagaacatggaacaactgcaaccataacacctcaagctcccacgtcggaaatacagctgacagactacggagctctaacattggattgttcacctagaacagggctagactttaatgagatggtgttgttgacaatggaaaaaaaatcatggctcgtccacaaacaatggtttctagacttaccactgccttggacctcgggggcttcaacatcccaagagacttggaatagacaagacttgctggtcacatttaagacagctcatgcaaaaaagcaggaagtagtcgtactaggatcacaagaaggagcaatgcacactgcgttgactggagcgacagaaatccaaacgtctggaacgacaacaatttttgcaggacacctgaaatgcagactaaaaatggataaactgactttaaaagggatgtcatatgtaatgtgcacagggtcattcaagttagagaaggaagtggctgagacccagcatggaactgttctagtgcaggttaaatacgaaggaacagatgcaccatgcaagatccccttctcgtcccaagatgagaagggagtaacccagaatgggagattgataacagccaaccccatagtcactgacaaagaaaaaccagtcaacattgaagcggagccaccttttggtgagagctacattgtggtaggagcaggtgaaaaagctttgaaactaagctggttcaagaagggaagcagtatagggaaaatgtttgaagcaactgcccgtggagcacgaaggatggccatcctgggagacactgcatgggacttcggttctataggaggggtgttcacgtctgtgggaaaactgatacaccagatttttgggactgcgtatggagttttgttcagcggtgtttcttggaccatgaagataggaatagggattctgctgacatggctaggattaaactcaaggagcacgtccctttcaatgacgtgtatcgcagttggcatggtcacgctgtacctaggagtcatggttcaggcggactcgggatgtgtaatcaactggaaaggcagagaactcaaatgtggaagcggcatttttgtcaccaatgaagtccacacctggacagagcaatataaattccaggccgactcccctaagagactatcagcggccattgggaaggcatgggaggagggtgtgtgtggaattcgatcagccactcgtctcgagaacatcatgtggaagcaaatatcaaatgaattaaaccacatcttacttgaaaatgacatgaaatttacagtggtcgtaggagacgttagtggaatcttggcccaaggaaagaaaatgattaggccacaacccatggaacacaaatactcgtggaaaagctggggaaaagccaaaatcataggagcagatgtacagaataccaccttcatcatcgacggcccaaacaccccagaatgccctgataaccaaagagcatggaacatttgggaagttgaagactatggatttggaattttcacgacaaacatatggttgaaattgcgtgactcctacactcaagtgtgtgaccaccggctaatgtcagctgccatcaaggatagcaaagcagtccatgctgacatggggtactggatagaaagtgaaaagaacgagacttggaagttggcaagagcctccttcatagaagttaagacatgcatctggccaaaatcccacactctatggagcaatggagtcctggaaagtgagatgataatcccaaagatatatggaggaccaatatctcagcacaactacagaccaggatatttcacacaaacagcagggccgtggcacttgggcaagttagaactagattttgatttatgtgaaggtaccactgttgttgtggatgaacattgtggaaatcgaggaccatctcttagaaccacaacagtcacaggaaagacaatccatgaatggtgctgtagatcttgcacgttaccccccctacgtttcaaaggagaagacgggtgctggtacggcatggaaatcagaccagtcaaggagaaggaagagaacctagttaagtcaatggtctctgcagggtcaggagaagtggacagtttttcactaggactgctatgcatatcaataatgatcgaagaggtaatgagatccagatggagcagaaaaatgctgatgactggaacattggctgtgttcctccttctcacaatgggacaattgacatggaatgatctgatcaggctatgtatcatggttggagccaacgcttcagacaagatggggatgggaacaacgtacctagctttgatggccactttcagaatgagaccaatgttcgcagtcgggctactgtttcgcagattaacatctagagaagttcttcttcttacagttggattgagtctggtggcatctgtagaactaccaaattccttagaggagctaggggatggacttgcaatgggcatcatgatgttgaaattactgactgattttcagtcacatcagctatgggctaccttgctgtctttaacatttgtcaaaacaactttttcattgcactatgcatggaagacaatggctatgatactgtcaattgtatctctcttccctttatgcctgtccacgacttctcaaaaaacaacatggcttccggtgttgctgggatctcttggatgcaaaccactaaccatgtttcttataacagaaaacaaaatctggggaaggaaaagctggcctctcaatgaaggaattatggctgttggaatagttagcattcttctaagttcacttctcaagaatgatgtgccactagctggcccactaatagctggaggcatgctaatagcatgttatgtcatatctggaagctcggccgatttatcactggagaaagcggctgaggtctcctgggaagaagaagcagaacactctggtgcctcacacaacatactagtggaggtccaagatgatggaaccatgaagataaaggatgaagagagagatgacacactcaccattctcctcaaagcaactctgctagcaatctcaggggtatacccaatgtcaataccggcgaccctctttgtgtggtatttttggcagaaaaagaaacagagatcaggagtgctatgggacacacccagccctccagaagtggaaagagcagtccttgatgatggcatttatagaattctccaaagaggattgttgggcaggtctcaagtaggagtaggagtttttcaagaaggcgtgttccacacaatgtggcacgtcaccaggggagctgtcctcatgtaccaagggaagagactggaaccaagttgggccagtgtcaaaaaagacttgatctcatatggaggaggttggaggtttcaaggatcctggaacgcgggagaagaagtgcaggtgattgctgttgaaccggggaagaaccccaaaaatgtacagacagcgccgggtaccttcaagacccctgaaggcgaagttggagccatagctctagactttaaacccggcacatctggatctcctatcgtgaacagagagggaaaaatagtaggtctttatggaaatggagtggtgacaacaagtggtacctacgtcagcgccatagctcaagctaaagcatcacaagaagggcctctaccagagattgaggacgaggtgtttaggaaaagaaacttaacaataatggacctacatccaggatcggggaaaacaagaagatatcttccagccatagtccgtgaggccataagaaggaacgtgcgcacgctagtcttagctcccacaagagttgtcgcttctgaaatggcagaggcgctcaagggaatgccaataaggtatcagacaacagcagtgaagagtgaacacacaggaaaagagatagttgaccttatgtgtcacgccactttcactatgcgtctcctgtctcctgtgagagttcccaattataatatgattatcatggatgaagcacattttaccgatccagccagcatagcagccagagggtatatctcaacccgagtgggtatgggtgaagcagctgcgattttcatgacagccactccccccggatcggtggaggcctttccacagagcaatgcagttatccaagatgaggaaagagacattcctgaaagatcatggaactcaggctatgactggatcactgatttcccaggtaaaacagtctggtttgttccaagcatcaaatcaggaaatgacattgccaactgtttaagaaagaatgggaaacgggtggtccaattgagcagaaaaacttttgacactgagtaccagaaaacaaaaaataacgactgggactatgttgtcacaacagacatatccgaaatgggagcaaacttccgagccgacagggtaatagacccgaggcggtgcctgaaaccggtaatactaaaagatggcccagagcgtgtcattctagccggaccgatgccagtgactgtggctagcgccgcccagaggagaggaagaattggaaggaaccaaaataaggaaggcgatcagtatatttacatgggacagcctctaaacaatgatgaggaccacgcccattggacagaagcaaaaatgctccttgacaacataaacacaccagaagggattatcccagccctctttgagccggagagagaaaagagtgcagcaatagacggggaatacagactacggggtgaagcgaggaaaacgttcgtggagctcatgagaagaggagatctacctgtctggctatcctacaaagttgcctcagaaggcttccagtactccgacagaaggtggtgctttgatggggaaaggaacaaccaggtgttggaggagaacatggacgtggagatctggacaaaagaaggagaaagaaagaaactacgaccccgctggctggatgccagaacatactctgacccactggctctgcgcgaattcaaagagttcgcagcaggaagaagaagcgtctcaggtgacctaatattagaaatagggaaacttccacaacatttaacgcaaagggcccagaacgccttggacaatctggttatgttgcacaactctgaacaaggaggaaaagcctatagacacgccatggaagaactaccagacaccatagaaacgttaatgctcctagctttgatagctgtgctgactggtggagtgacgttgttcttcctatcaggaaggggtctaggaaaaacatccattggcctactctgcgtgattgcctcaagcgcactgctatggatggccagtgtggaaccccattggatagcggcctctatcatactggagttctttctgatggtgttgcttattccagagccggacagacagcgcactccacaagacaaccagctagcatacgtggtgataggtctgttattcatgatattgacagcggcagccaatgagatgggattactggaaaccacaaagaaggacctggggattggtcatgcagctgctgaaaaccaccatcatgctgcaatgctggacgtagacctacatccagcttcagcctggactctctatgcagtggccacaacaattatcactcccatgatgagacacacaattgaaaacacaacggcaaatatttccctgacagctattgcaaaccaggcagctatattgatgggacttgacaagggatggccaatatcaaagatggacataggagttccacttctcgccttggggtgctattctcaggtgaacccgctgacgctgacagcggcggtatttatgctagtggctcattatgccataattggacccggactgcaagcaaaagctactagagaagctcaaaaaaggacagcagccggaataatgaaaaacccaactgtcgacgggatcgttgcaatagatttggaccctgtggtttacgatgcaaaatttgaaaaacagctaggccaaataatgttgttgatactttgcacatcacagatcctcctgatgcggaccacatgggccttgtgtgaatccatcacactagccactggacctctgactacgctttgggagggatctccaggaaaattctggaacaccacgatagcggtgtccatggcaaacatttttaggggaagttatctagcaggagcaggtctggccttttcattaatgaaatctctaggaggaggtaggagaggcacgggagcccaaggggaaacactgggagaaaaatggaaaagacagctaaaccaattgagcaagtcagaattcaacacttacaaaaggagtgggattatagaggtggatagatctgaagccaaagaggggttaaaaagaggagaaccgactaaacacgcagtgtcgagaggaacggccaaactgaggtggtttgtggagaggaaccttgtgaaaccagaagggaaagtcatagacctcggttgtggaagaggtggctggtcatattattgcgctgggctgaagaaagtcacagaagtgaaaggatacacgaaaggaggacctggacatgaggaaccaatcccaatggcaacctatggatggaacctagtaaagctatactccgggaaagatgtattctttacaccacctgagaaatgtgacaccctcttgtgtgatattggtgagtcctctccgaacccaactatagaagaaggaagaacgttacgtgttctaaagatggtggaaccatggctcagaggaaaccaattttgcataaaaattctaaatccctatatgccgagtgtggtagaaactttggagcaaatgcaaagaaaacatggaggaatgctagtgcgaaatccactctcaagaaactccactcatgaaatgtactgggtttcatgtggaacaggaaacattgtgtcagcagtaaacatgacatctagaatgttgctaaatcgattcacaatggctcacaggaagccaacatatgaaagagacgtggacttaggcgctggaacaagacatgtggcagtagaaccagaggtggccaacctagatatcattggccagaggatagagaatataaaaaatggacacaaatcaacatggcactatgatgaggacaatccatacaaaacatgggcctatcatggatcatatgaggtcaagccatcaggatcagcctcatccatggtcaatggtgtggtgagactgctaaccaaaccatgggatgtcattcccatggtcacacaaatagccatgactgacaccacaccctttggacaacagagggtgtttaaagagaaagttgacacgcgtacaccaaaagcgaaacgaggcacagcacaaattatggaggtgacagccaggtggttatggggttttctctctagaaacaaaaaacccagaatctgcacaagagaggagttcacaagaaaagtcaggtcaaacgcagctattggagcagtgttcgttgatgaaaatcaatggaactcagcaaaagaggcagtggaagatgaacggttctgggaccttgtgcacagagagagggagcttcataaacaaggaaaatgtgccacgtgtgtctacaacatgatgggaaagagagagaaaaaattaggagagttcggaaaggcaaaaggaagtcgcgcaatatggtacatgtggttgggagcgcgctttttagagtttgaagcccttggtttcatgaatgaagatcactggttcagcagagagaattcactcagtggagtggaaggagaaggactccacaaacttggatacatactcagagacatatcaaagattccagggggaaatatgtatgcagatgacacagccggatgggacacaagaataacagaggatgatcttcagaatgaggccaaaatcactgacatcatggaacctgaacatgccctattggccacgtcaatctttaagctaacctaccaaaacaaggtagtaagggtgcagagaccagcgaaaaatggaaccgtgatggatgtcatatccagacgtgaccagagaggaagtggacaggttggaacctatggcttaaacaccttcaccaacatggaggcccaactaataagacaaatggagtctgagggaatcttttcacccagcgaattggaaaccccaaatctagccgaaagagtcctcgactggttgaaaaaacatggcaccgagaggctgaaaagaatggcaatcagtggagatgactgtgtggtgaaaccaatcgatgacagatttgcaacagccttaacagctttgaatgacatgggaaaggtaagaaaagacataccgcaatgggaaccttcaaaaggatggaatgattggcaacaagtgcctttctgttcacaccatttccaccagctgattatgaaggatgggagggagatagtggtgccatgccgcaaccaagatgaacttgtaggtagggccagagtatcacaaggcgccggatggagcttgagagaaactgcatgcctaggcaagtcatatgcacaaatgtggcagctgatgtacttccacaggagagacttgagattagcggctaatgctatctgttcagccgttccagttgattgggtcccaaccagccgtaccacctggtcgatccatgcccaccatcaatggatgacaacagaagacatgttgtcagtgtggaatagggtttggatagaggaaaacccatggatggaggacaagactcatgtgtccagttgggaagacgttccatacctaggaaaaagggaagatcgatggtgtggatccctaataggcttaacagcacgagccacctgggccaccaacatacaagtggccataaaccaagtgagaaggctcattgggaatgagaattatctagacttcatgacatcaatgaagagattcaaaaacgagagtgatcccgaaggggcactctggtaagccaactcattcacaaaataaaggaaaataaaaaatcaaacaaggcaagaagtcaggccggattaagccatagcacggtaagagctatgctgcctgtgagccccgtccaaggacgtaaaatgaagtcaggccgaaagccacggttcgagcaagccgtgctgcctgtagctccatcgtggggatgtaaaaacccgggaggctgcaaaccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccaaaaaatggaatggtgctgttgaatcaacaggttct

SEQ ID NO: 6 - DNA, 10723 bp, Dengue virus type 2
  misc_feature (7570)..(10290): Non-structural protein 5 sequence in Dengue virus type 2 strain TSV01, cDNA of complete RNA genome (gi 14585842; gbAY037116.1)
agttgttagtctacgtggaccgacaaagacagattctttgagggagctaagcttaacgtagttctaacagtttttgaattagagagcagatctctgatgaataaccaacggaaaaaggcgagaaatacgcctttcaatatgctgaaacgcgagagaaaccgcgtgtcaactgtgcagcagctgacaaagagattctcacttggaatgctacagggacgaggaccattgaaactgttcatggccctggtggcattccttcgtttcctaacaatcccgccaacagcagggatattaaaaagatggggaacaatcaaaaaatcaaaggctatcaatgtcttgagagggttcaggaaagagattggaaggatgctgaacatcttgaacaggagacgcagaactgcaggtataattattatgatgattccaacagtgatggcgttccatttaaccacacgcaacggagaaccacacatgatcgtcagtagacaagagaaggggaaaagtcttctgtttaaaacagagaacggtgtgaacatgtgtaccctcatggccatggaccttggtgaattgtgtgaagacacaatcacttataattgtcctcttctcaggcagaatgaaccagaagacatagactgtgggtgccactctacgtctacatgggtaacttatgggacatgcaccgccacaggagaacacagaagggaaaaaagatcagtggcactcgttccacatgtgggaatgggactggagacacgaactgaaacatggatgtcatcagaaggggcctggaaacatgcccagagaattgaaacttgggtcttgagacatccaggcttcaccataatggcagcaatcctggcatacaccataggaacgacatatttccaaagagtcctgattttcatcttactgacagctgtcactccttcaatgacaatgcgttgtataggaatatcaaatagggactttgtggaaggggtttcaggaggaagctgggttgacatagtcttggaacatggaagctgtgtgacgacgatggcgaaaaataaaccaacattggattttgaactggtaaaaacagaagccaaacatcccgccactctaaggaagtattgtatagaagcaaagctgaccaacacaacaacagcatctcgctgcccaacacaaggagaacctagcctaaatgaagaacaggacaaaagatttgtctgcaaacactccatggtagacagaggatggggaaatggatgcggattatttggaaagggaggcatcgtgacctgtgcaatgttcacatgcaaaaagaacatggaaggaaaagtcgtgcaaccagaaaacttggagtacaccattgtgataacacctcactcaggggaagagaatgcagtcggaaatgacacaggaaaacacggcaaggaaattaaagtaacaccacagagttccatcacagaagcagaactaacaggctatggcactgtcacgatggaatgctctccgagaacgggcctcgacttcaatgagatggtgttgctgcaaatggaaaacaaggcttggctggtgcacaggcaatggttcttagacctgccgttaccatggctgcccggagcagacacacaaggatcaaattggatacagaaggagacattggtcactttcaaaaatccccatgcaaagaaacaggatgttgttgttttaggatcccaagaaggggctatgcatacagcactcacaggggccacggaaatccagatgtcatcaggaaacttactgttcacaggacatcttaagtgcaggctgagaatggacaaactacagctcaaaggaatgtcatattctatgtgtacaggaaagtttaaagttgtgaaggaaatagcagaaacacaacatggaacaatagttatcagagtacaatatgaaggggacggttctccgtgcaagatcccttttgaaataatggatttggaaaaaagacatgtcttaggtcgcttgattacagtcaacccaattgttacagaaaaagacagcccagtcaacatagaagcagaacctccattcggagacagctacatcattataggagtagaaccgggacaactgaagctcagctggtttaagaaaggaagttctattggccaaatgtttgagacaacaatgagaggagcgaagagaatggccattttaggtgacacagcttgggattttggatccctgggaggagtgttcacatctataggaaaggccctccaccaagtctttggagcaatctatggggctgccttcagtggggtttcatggactatgaaaatcctcataggagtcgtcatcacatggataggaatgaattcacgcagcacctcactgtctgtgtcactagtattagtgggggtcgtgacattatatttgggagttatggtgcaggccgatagtggctgcgttgtgagttggaaaaacaaagaactgaaatgtggcagtgggatttttatcacagacaacgtgcacacatggacagaacaatacaaattccaaccagaatccccttcaaagctggcttcagctatccagaaggctcatgaagagggcatttgtggaatccgctcagtaacaagactggagaatctgatgtggaaacaaataacaccagaactgaatcacattctatcagaaaatgaggtaaagttgactatcatgacaggagacattaaaggaatcatgcaggcaggaaaacgatccctgcggcctcaacccactgagctgaagtactcttggaaagcatggggcaaagcgaaaatgctctccacagagcttcataaccacacctttctcattgatggccccgaaacagcagaatgtcccaacacaaacagagcttggaactcactagaagttgaagactatggctttggagtattcaccaccaacatatggctgaaattgaaagaaaggcaggatgtattttgtgactcaaaactcatgtcagcagccataaaagacaacagagccgtccacgccgatatgggttattggatagaaagcgcactcaatgacacatggaagattgagaaagcctcttttattgaagttaaaagctgccactggccaaagtcacacactctctggagtaatggagtgctagaaagtgagatgataattccaaagaattttgcaggaccagtgtcacaacacaattacagaccaggctatcatacacaaacggcaggaccctggcatctaggtaggcttgagatggactttgatttctgcgaaggaaccacagtggtagtgactgaggactgtggaaacagaggaccctctttaagaacaactactgcttctggaaaactcataacagaatggtgctgccgatcctgcacattaccaccgctaaggtacagaggtgaggatggatgctggtatggaatggaaatcagaccattgaaagagaaagaagagaacttggtcaactctttggtcacagccggacatggacagattgacaacttctcactaggagtcttgggaatggcattgttcctggaagagatgctcaggacccgagtaggaacgaaacatgcaatattactagttgcagtttctttcgtgacattgatcacagggaacatgtcctttcgagatttggggagagtgatggtcatggtgggcgctactatgacggatgacataggcatgggcgtgacttatctcgccctattagcagccttcaaagtcagaccaactttcgcagctggactactcttaagaaagctgacctccaaggaattgatgatgaccaccataggaatcgtactcctctctcagagcaccataccagagactatacttgaactgactgacgcgtgggctttggggatgatggttctcaaaatagtgagaaacatggaaaagtatcaactagcagtgactatcatggccatcttgtgcgtcccaaatgcagtgatattacaaaacgcatggaaagtgagctgcacaacactggcagtggtgtccgtttccccactgcttttaacatcctcacagcagaaagcggattggataccactggcgttgacgatcaaaggcctcaatccaacagccattttcttaacaaccctctcaagaactagcaagaaaaggagctggccactaaatgaggctattatggcagtcgggatggtgagcattttagccagttctctcttaaagaatgatattcccatgacaggaccattagtggctggagggcttctcactgtgtgttacgtgctcactggaagatcggctgatttggaactggagagagctgctgacgtaagatgggaagaacaggcagagatatcaggaagtagtccaattctgtcaataacaatatcggaagatggtagcatgtcgataaaaaatgaagaagaagaacaaacactgaccatactcattagaacaggactgctggtgatatcaggactttttcccgcgtcaataccaatcacggcagctgcatggtacctgtgggaagtgaaaaaacaacgagccggagtattgtgggatgtcccttcacccccacctgtgggaaaggccgaactggaagatggagcctatagaatcaagcagaaagggattctaggatactcgcagatcggagccggagtttacaaagaaggaacattccacacaatgtggcatgtcacacgtggtgctgtcctaatgcataaagggaagagaattgaaccatcatgggcggacgtcaagaaagacctaatatcgtatggaggaggctggaagctagaaggagaatggaaggaaggagaagaagtccaggtcctggcattagagcctggaaagaatccaagagccgtccaaacaaaacccggtctttttaaaactaacactggaaccataggcgccgtatctctggacttctctcctggaacgtcaggatctccaattgtcgacaaaaaaggaaaagttgtgggcctttatggcaacggtgtcgtcacaaggagtggagcatatgtgagtgccatagcccagactgaaaaaagcatcgaagacaatccagagattgaagatgacatctttcgaaagaaaagattgaccatcatggacctccacccaggagcgggaaaaacaaagagataccttccagcaatagttagagaagccataaaacgaggcttgagaacactaatcctggcccccactagagttgtggcggctgaaatggaagaagctctcagaggacttccaataagataccaaaccccagctatcagagctgagcacactgggcgggagattgtggatctaatgtgtcacgccacatttaccatgaggctgctatcaccaattagagtgccaaattacaacctgatcatcatggatgaagcccatttcacagacccagcaagcatagcagctagaggatacatttcaactcgagtagagatgggtgaagcagctgggattttcatgacagctactcctcctggaagcagagacccatttcctcagagcaatgcaccaatcatggatgaagaaagggaaatccctgagcgttcgtggaactctggacatgagtgggttacggattttaaagggaagactgtttggtttgttccaagtataaaagcaggaaatgatatagcagcttgcctgagaaagaatggaaagaaagtgatacaactcagcaggaagacctttgattctgaatatatcaagactaggaccaatgattgggactttgtggtcacgacagacatttcagaaatgggtgctaacttcaaggctgagagggttatagaccccagacgctgcatgaaaccagtcatactaactgacggtgaagagcgggtgatcttggcaggacccatgccagtgacccactctagtgcagcacaaagaagagggagagtaggaagaaatccaaaaaatgaaaatgaccagtacatatacatgggggaacctctggaaaatgatgaagactgtgcacactggaaagaagctaagatgcttctagataacatcaacacgcctgaaggaatcattcccagcatgttcgaaccagagcgtgaaaaggtggatgccattgatggtgaataccgcttgagaggagaagcgaggaaaacttttgtggacctaatgagaagaggagacctaccagtctggctagcctacagagtggctgctgaaggtatcaactacgcagacagaagatggtgctttgatggagtcaagaacaaccaaatcttggaagaaaatgtggaagtggaaatttggacaaaagaaggagaaaggaagaaattaaaacccagatggttggatgccaggatctactctgacccactggcgctcaaagaattcaaggaattcgcagctggaagaaagtccctgactctgaatctaatcacagagatgggtaggctcccaaccttcatgacccagaaagcaaggaatgcactggacaacttagcagtcctgcatacggctgaagcaggcggaagggcgtacaatcatgctcttagtgaactgccggagaccctggagacattgcttttactgacacttttggccacagtcacgggcggaatcttcctgttcttgatgagcggaaagggtatagggaagatgaccctgggaatgtgctgcataatcacggccagtattctcctatggtatgcacaaatacagccacactggatagcagcctcaataatactggagttttttctcatagtcttgctcattccagaaccagaaaagcagagaacaccccaagataaccaattaacttatgttgtcatagccatccttacagtggtggccgcaaccatggcaaacgagatgggtttcctggaaaaaacaaagaaagatttcggattgggaagcattgcaacccagcaacctgagagcaacatcctggacatagatctacgtcctgcatcagcttggactctatatgccgtggcaacaactttcatcacaccaatgttgagacatagcattgaaaattcctcagtgaatgtgtctctaacagccattgccaaccaagccacagtgttaatgggtcttgggaaaggatggccattgtcaaaaatggacatcggagttccccttctcgctatcgggtgctattcacaagtcaaccccataactctcacggcagcccttctcttattggtagcacattatgctatcatagggccaggactccaagcgaaagcaactagagaggctcagaaaagagcagcagcgggcatcatgaaaaacccaactgtggatggaataacagtgattgacctagatccaataccctatgatccaaagtttgaaaagcagttgggacaagtaatgctcttagtcctctgcgtgacccaagtattgatgatgaggactacatgggctttatgtgaagctctaaccttagcgaccgggcccatctctacactgtgggaaggaaatccagggagattttggaatacaactattgcagtgtcaatggctaacatttttagagggagttacctggccggagccggacttctcttttctatcatgaagaacacggccaacacaagaaggggaactggcaacacaggagagacgcttggagagaaatggaaaaaccggttgaatgcattggggaagagtgaattccagatctataagaaaagtggaatccaggaagtggatagaaccttagcaaaagaaggcatcaaaagaggagaaacggaccatcacgctgtgtcgcgaggctcggcgaaactgagatggttcgtcgagagaaacctggtcacaccagaagggaaagtagtggaccttggctgcggcagggggggctggtcatactattgtgggggactaaagaatgtaaaagaagtcaaaggcctaacaaaaggaggaccaggacacgaagaacccattcccatgtcaacatatggttggaatctggtgcgtcttcaaagtggagttgatgttttttttactccgccagaaaagtgtgacacattactgtgtgacataggggagtcgtcaccaaaccccacggttgaggcaggacgaacactcagagttctaaacttagtggaaaattggctgaacaacaacacccaattttgcataaaggttctcaacccatatatgccctcagttatagaaaaaatggaagcgctacaaaggaaatacggaggagctttggtgaggaatccactctcacgaaattccacacacgagatgtactgggtatccaatgcttccgggaacatagtgtcatcagtgaacatgatttcaagaatgttgattaacagattcacaatgagacataagaaggccacatacgagccagatgttgacctcggaagcggaacccgcaacatcggaattgaaagtgagacaccaaatttagacataattgggaaaagaatagagaaaataaaacaagagcatgaaacatcatggcactatgaccaagaccacccatacaaaacgtgggcctaccatggcagctacgaaacaaaacagactggatcagcatcatccatggtgaacggagtggttaggctgctaacaaaaccttgggacatcatccctatggtgacacagatggcaatgacagacacgactccatttgggcaacagcgcgttttcaaagagaaagtggacacgagaacccaagaaccgaaagaaggcacgaaaaaactaatgaaaatcacggcagaatggctctggaaagaactaggaaagaaaaagacacctaggatgtgcaccagagaagaattcacaagaaaggtgagaagcaatgcagccttaggtgccatattcactgatgagaacaagtggaagtcggcacgtgaggctgttgaagatagtggattttgggaattggttgacaaggaaaggaatctccatcttgaaggaaagtgtgagacatgtgtgtataacatgatgggaaagagagagaagaagctaggggagttcggcaaagcaaaaggcagcagagccatatggtacatgtggcttggagcacgcttcttagagtttgaagccctaggattcttgaatgaagatcactggttttccagagagaactccctgagtggagtggaaggagaagggctgcacaaactaggctacattttaagagacgtgagtaagaaagaaggaggagcaatgtacgccgatgacaccgcaggatgggacacaagaatcacactagaggacttaaaaaatgaagaaatggtgacaaaccacatggaaggagaacacaagaaacttgctgaagccattttcaaattaacgtaccaaaacaaggtggtgcgtgtgcaaagaccaacaccaagaggcacagtaatggacattatatcgagaagagaccaaagaggtagtggacaagttgtcacctacggcctcaatactttcaccaacatggaagcccaactgatcagacagatggagggagaaggagtcttcaaaagcatccagcacctgacagtcacagaagaaattgcagtgaaaaactggttagtaagagtggggcgtgagaggttatcaagaatggccatcagtggagatgattgtgttgtgaaacccttagatgacaggtttgcaagcgctctaacagctctaaatgacatgggaaaggttaggaaagacatacaacaatgggaaccttcaagaggatggaacgattggacacaagtgcccttttgttcacaccatttccatgagttaatcatgaaggacggtcgtgtactcgtagttccatgtagaaaccaagatgaactgattggtagggcccgaatttcccagggagccgggtggtccttgcgggaaacagcctgtttggggaagtcttacgcccaaatgtggagcctgatgtacttccacagacgtgaccttaggctggcggcaaatgccatttgctcggcagtcccatcacattgggttccaacaagtcgaacaacctggtccatacacgccacacatgagtggatgacaacagaagacatgctgacagtctggaacagggtgtggattcaagaaaacccatggatggaagacaaaactccagtagaatcatgggaggaaatcccatatttggggaaaagagaagaccaatggtgcggctcattgattgggctaacaagcagggccacctgggcaaagaacatccaaacagcaataaatcaagttagatccctaataggcaatgaggaatacacagactacatgccatccatgaaaagattcagaagagaagaggaagaggcaggagtcttgtggtagaaagcaggaacatcatgagacaaagtcagaagtcaggtcggattaagccatagtacggaaaaaactatgctacctgtgagccccgtccaaggacgttaaaagaagtcaggccactacaagtgccatagcttgagtaaactatgcagcctgtagcttcacctgagaaggtgtaaaaaatctgggaggccacaaaccatggaagctgtacgcatggcgtagtggactagcggttagaggagacccctcccttacaaatcgcagcaacaatgggggcccaaggtgagatgaagctgtagtctcactgaaaggactagaggttagaggagacccccccgaaataaaaaacagcatattgacgctgggaaagaccagagatcctgctgtctcctcagcatcattccaggcacagaacgccagaaaatggaatggtgctgttgaatcaacaggttct

SEQ ID NO: 7 - DNA, 10706 bp, Dengue virus type 3
  misc_feature (7631)..(10290): Non-structural protein 5 in Dengue virus type 3, isolate D3MY05-34640, cDNA of complete genomic RNA (gi 255031230; emb FN429918.1)
agttgttagtctacgtggaccgacaagaacagtttcgactcggaagcttgcttaacgtagtactgacagtttttattagagagcagatctctgatgaacaaccaacggaaaaagacgggaaaaccgtctatcaatatgctgaaacgcgtgagaaaccgtgtgtcaactggatcacagttggcgaagagattctcaagaggattgctgaatggccaaggaccaatgaaattggttatggcgttcatagctttcctcagatttctagccattccaccgacagcgggaatcttggctagatggggaaccttcaagaagtcgggggctattaaggtcctgagaggcttcaagaaggagatctcaaatatgctgagcattatcaacagacggaaaaagacatcgctctgtctcatgatgatgttaccagcaacacttgctttccacttaacttcacgagatggagagccgcgcatgattgtggggaagaatgaaagaggaaaatccctactttttaagacagcctctggaatcaacatgtgcacactcatagccatggatttgggagaaatgtgtgatgacacggtcacttacaaatgccccctcattactgaagtggagcctgaagacattgactgctggtgcaacctcacatcgacgtgggtgacctacggaacgtgcaaccaagctggagagcatagacgcgataagagatcggtggcgttagctcctcacgtcggcatgggactggacacacgcgcccaaacctggatgtcggctgagggagcttggagacaagtcgagaaggtagagacatgggcctttaggcacccagggttcacaatactagccctatttcttgcccattacataggcacttccttgacccagaaagtggtcattttcatactactaatgctggtcaccccatccatgacaatgagatgtgtgggagtaggaaacagagattttgtggaaggcctatcaggagccacgtgggttgacgtggtgctcgaacatggtgggtgtgtgaccaccatggctaagaacaagcccacgctggacatagagctccagaagaccgaggccacccaactggcaaccttaaggaaactatgcattgagggaaaaatcaccaacgtgacaaccgactcaaggtgccccacccaaggggaagcgattctacctgaggagcaggaccagaactacgtgtgtaagcacacatacgtggacagaggctggggaaacggttgtggtttgtttggcaagggaagcttggtaacatgcgcaaaattccaatgtttggaattaatagagggaaaagtggtgcagcatgagaacctcaaatacaccgtcatcatcacagtgcacacaggagaccaacaccaggtgggaaatgaaacgcagggagtcacggctgagataacaccccaggcatcaaccgtcgaagccatcttacctgaatatggaacccttgggctagaatgctcacctcggacaggtctggatttcaatgaaatgattttgttgacaatgaagaacaaagcatggatggtacatagacaatggttttttgacctacctttaccatggacatcaggggctacaacagaaacaccaacctggaataagaaagagcttcttgtgacattcaaaaacgcacacgcaaaaaaacaagaagtagttgtccttggatcgcaagagggagcaatgcacacagcactgacaggagctacagagatccaaacctcaggaggcacaagtatttttgcggggcacttgaaatgcagactcaagatggacaaattggaactcaaggggatgagctatgcaatgtgctcaaatgcctttgtgttgaagaaagaagtctctgaaacacaacatgggacaatactcattaaggtcgagtacaaaggagaagacgcaccttgcaagattcctttctccacggaggatggacaagggaaagcccacaatggcagactgatcacagctaacccagtggtgaccaagaaggaggaacctgtcaatattgaggcagaacctccttttggggaaagtaacataataattggaactggagacaaagccttgaaaatcaattggtacaagaagggaagctcgattgggaagatgttcgaggccactgccagaggtgcaaggcgcatggccatcttgggagacacagcctgggactttggatcagtaggtggtgttttaaattcactagggaaaatggtgcaccaaatatttggaagtgcttacacggccctttttagtggagtctcctggataatgaaaattggaataggtgtccttttaacttggatagggttgaattcaaaaaacacttctatgtcattttcatgcattgtgataggaatcatcacactctatctgggagccgtggtgcaagctgacatggggtgtgtcataaactggaaaggcaaagaactcaaatgtggaagtggaattttcgttactaatgaggtccacacctggacagagcaatacaaatttcaagcagactcccccaaaagactggcgacagccattgcaggcgcctgggagaatggagtgtgcggaatcaggtcgacaaccagaatggagaacctcttgtggaagcaaatagccaatgaactaaactacatattatgggaaaacaacatcaaattgacggtagttgtgggtgatataattgggatcttagagcaagggaaaagaacactaacaccacaacccatggagctaaaatattcatggaaaacatggggaaaggcgaaaatagtgacagctgaaatacaaaattcctcctttataatagatgggccaaacacaccagagtgtccaaatgcctcaagagcatggaatgtgtgggaggtggaagattacggcttcggagtcttcacaaccaatatatggctgaaactccgagaaatgtacacccaactatgtgaccacaggctaatgtcggcagccgttaaggatgagagagccgtacacgccgacatgggctattggatagaaagccaaaagaatggtagttggaagctagaaaaggcatctctcatagaggtgaaaacctgcacatggccaaaatcacacactctctggagcaatggtgtgttggagagtgacatgatcatcccaaagagtctggctggtcctatctcacaacacaattacaggcctggataccacacccaaacggcaggaccatggcacctaggaaaattggagctggacttcaactattgcgaaggaacaacagttgtcatcacagaaaattgtgggacaagaggcccatcactgaggacaacaacagtgtcagggaagttgatacacgaatggtgttgccgctcgtgtacacttcctcccctgcgatatatgggagaagacggctgctggtatggcatggaaattagacccattaatgagaaagaagagaacatggtaaagtctttagtctcagcagggagtggaaaggtggataacttcacaatgggtgtcttgtgtcttgcaatcctctttgaagaggtaatgagaggaaaattcgggaaaaagcacatgattgcaggggttctcttcacatttgtgctccttctctcagggcaaataacatggagagacatggcgcgcacactcataatgattggatccaacgcctctgacagaatgggaatgggcgttacttacctagcattgattgcaacatttaaaattcagccatttttggctttgggattcttcctgaggaaactgacatccagagaaaatttattattgggagttgggttggccatggcaacaacgttgcaactgccagaggacattgaacaaatggcgaatggaatagccttggggctcatggctctcaaattgataacacaatttgaaacataccaactatggacggcattagtctccctaatgtgttcaaacacaattttcacgttgactgttgcctggagaacagccaccctgattttggccggaatttcgcttttgccagtgtgccagtcttcgagcatgaggaaaacagattggctcccaatgactgtggcagctatgggagttccacctctaccactttttattttcagtttgaaagacacactcaaaaggagaagctggccactgaatgagggggtgatggccgttggacttgtgagcattctagcaagttctctccttaggaatgacgtacccatggctggaccattagtggctgggggcttgctgatagcgtgctacgtcataactggcacgtcagcagacctcactgtagaaaaagcagcagatgtgacatgggaggaagaggctgagcaaacaggagtgtcccacaatttaatgatcacagttgatgatgatggaacaatgagaataaaagatgatgagactgagaacatcttaacagtgcttttgaaaacagcattactaatagtgtcaggcatttttccatactccatacctgcaacacttttggtctggcacacttggcaaaagcaaacccaaagatccggcgtcctgtgggacgtacccagccccccagagacacagaaagcagaactggaagaaggggtctataggatcaagcagcaaggaatttttggaaagacccaagtgggggttggagtacaaaaagaaggagttttccacactatgtggcacgtcacaagaggagcagtgttgacatacaatggaaaaagactggaaccaaactgggctagcgtaaaaaaagatctgatttcatacggaggaggatggagactgagtgcacaatggcaaaaaggagaagaggtgcaggttattgccgtagagcctgggaagaacccaaagaactttcaaaccatgccaggcattttccagacaacaacaggggagataggagcgattgcactggacttcaagcctggaacttcaggatctcccatcataaacagagagggaaaggtagtgggactatatggcaatggagtggtcacaaagaatggtggctatgtcagtggaatagctcaaacaaacgcagaaccagacggaccgacaccagagttggaagaagagatgttcaaaaagcgaaatctaaccataatggatctccaccccgggtcaggaaagacgcggaaataccttccagctatagttagagaggcaatcaagagacgcttaaggactctaattttggcaccaacaagggtggttgcagctgagatggaagaagcattgaaggggctcccaataaggtatcaaacaactgcaacaaaatctgaacacacagggaaagagattgttgatctaatgtgtcacgcaacgttcacaatgcgtttgctgtcaccagtcagagttccaaactacaacttgataataatggatgaggctcatttcacagatccagccagcatagcggctagagggtacatatcaactcgtgtaggaatgggagaggcagccgcaattttcatgacagctacacctcctggaacagctgatgcctttcctcagagcaacgcaccaattcaagatgaagaaagagacataccagaacgctcatggaattcaggcaatgattggattaccgactttgccggaaagactgtgtggtttgtccccagcattaaagctgggaatgacatagcaaactgcttgcggaaaaatggaaaaaaggtcattcaacttagtaggaaaacttttgacacagaatatcaaaagactaaactaaatgattgggactttgtggtgacaacagacatttcagaaatgggagccaatttcaaagcagacagagtgatcgacccaagaagatgtctcaagccagtcattttgacagacggacccgagcgcgtgatcctggcaggaccaatgccagtcaccgtagcgagtgctgcgcaaaggagagggagagttggcaggaacccacaaaaagaaaatgaccaatacatattcacgggccagcctcttaacaatgatgaagaccacgctcactggacagaagcaaaaatgctgctagacaacatcaacacaccagaaggaatcataccagctctctttgaaccagaaagggagaagtcagccgccatagacggcgaataccgcctgaaaggtgagtccaggaagaccttcgtggaactcatgaggaggggtgacctcccagtttggctagctcataaagtagcatcagaaggaatcaaatacacagacagaaaatggtgttttgatggagagcgcaacaatcaaattttggaggagaacatggatgtggaaatctggacaaaggaaggagaaaagaaaaaattgaggcccaggtggcttgatgcccgcacttattcagatcccttagcgctcaaggaattcaaggactttgcggctggtagaaagtcgatcgcccttgatcttgtgacagaaataggaagagtaccttcacatttagctcacagaacgagaaacgctctggacaatctggtgatgttgcacacgtcagaacacggtgggagggcctacaggcatgcagtggaggaattaccagaaacaatggaaacactcttactcctgggactcatgatcctgttaacaggtggggcgatgcttttcttgatatcaggcaaaggagttggaaagacttcaataggactcatttgtgtagttgcttccagcggcatgttatggatggctgatattccactccaatggattgcgtcggccatagtcctggagttttttatgatggtgctacttataccagaaccagaaaagcagagaactccccaagacaatcaactcgcatatgtcgtgataggcatacttacattggctgcaatagtagcagccaatgaaatgggattgttggaaaccacaaagagagatttggggatgtccaaagaaccaggtgttgcttcgccaaccagctatttggatgtggatttgcacccagcatcagcctggacattgtacgctgtggccacaacagtaataacaccaatgttgagacataccatagagaattccacagcaaatgtgtccctggcagctatagccaaccaggcagtggtcctgatgggtttagacaaaggatggccgatatcgaaaatggacttaggcgtgccactattggcactgggttgctattcacaagtgaacccactaactctcacagcggcagtactcctgctagtcacgcattatgctattataggtccaggattgcaggcaaaagccactcgtgaagctcaaaaaaggacggctgctggaataatgaagaatccaacggtggatgggataatgacaatagacctagatcctgtaatatacgattcaaaatttgaaaaacaactaggacaagttatgctcctggtcctgtgtgcagttcaacttttgttaatgagaacatcatgggcattttgtgaagctctaaccctagccacaggaccaataacaacactctgggaaggatcacctgggaagttctggaacaccacgatagctgtttccatggcgaacatctttagagggagctatttagcaggagctgggcttgctttttctatcatgaaatcagttggaacaggaaagagaggaacagggtcacaaggtgaaactttgggagaaaagtggaaaaagaaattgaatcaattatcctggaaagagtttgacctctacaaaaaatccggaatcactgaagtggatagaatagaagccaaagaagggttgaaaagaggagaaataacacaccatgccgtatccagaggcagcgcaaaacttcaatggttcgtggagagaaacatggtcattcctgaaggaagagtcatagacctgggctgtggaagaggaggctggtcatattattgtgcaggattgaaaaaagttacagaagtgcgaggatacacaaaaggcggcccaggacatgaagagccagtacctatgtctacatatggatggaacatagtcaagctgatgagtggaaaggatgtgttttatcttccacctgaaaagtgtgacactctattgtgtgacattggagaatcttcaccaagcccaacagtggaggagagcagaaccataagagtcttgaaaatggttgaaccatggctaaaaaataaccagttttgcattaaagtattgaacccttacatgccagctgtgattgagcacctagaaagactacaaaggaaacatggaggaatgcttgtgagaaatccactctcacgaaactccacgcacgaaatgtactggatatctaatggcacaggcaacatcgtctcttcagtcaacatggtatccagactgctactgaacagattcacaatgacatataggaaacccaccatagagaaagatgtggatttaggagcggggacccgacatgtcaatgcggaaccagaaacgcccaacatggatgtcattggggaaagaataagaaggatcaaggaggagcatagttcaacatggcactatgatgatgaaaatccttacaaaacgtgggcttaccatggatcatatgaagttaaggccacaggctcagcctcctccatgataaatggagtcgtgaaactcctcacgaaaccatgggatgtggtgcccacggtgacacagatggcaatgacggacacgaccccttttggccagcaaagggtttttaaagagaaagtggacaccaggacacccaaacccatgccaggaacaagaaaggttatggagatcacagcggaatggctttggagaaccctgggaaggaacaaaagacccagattatgcacgagagaggagttcacaaaaaaggtcagaaccaacgcagctatgggcgccgtattcacagaggagaaccaatgggacagtgctagagctgccgttgaagatgaagaattctggaaactcgtggacagagaacgtgaactccacaaactgggcaagtgtggaagctgcgtttataacatgatgggcaagagagagaagaaacttggagagtttggcaaagcaaaaggcagtagagccatatggtacatgtggttgggagccaggtaccttgagttcgaggcactcggattcttaaatgaagaccattggttctcacgtgaaaactcttacagtggagtagaaggggaaggactgcataagctgggatacatcttgagagacatttccaagatacccggaggagctatgtatgctgatgacacagctgggtgggacacaagaataacagaagatgacctgcacaatgaggaaaaaatcacacagcaaatggatcctgaacacaggcagttagcaaacgctatatttaagctcacataccaaaacaaagtggtcaaagttcaacgaccaactccaaagggcacggtaatggacatcatatctaggaaagaccaaagaggcagtggacaggtgggaacttatgggctgaatacattcaccaacatggaagcccagttgatcagacaaatggaaggagaaggtgtgttgtcgaagacagacctagagaaccctcatctgctagagaagaaaattacacaatggttggaaaccaaaggagtggagaggttaaaaagaatggccatcagcggggatgattgcgtggtgaaaccaatcgacgacaggttcgccaatgccctgcttgccctgaatgacatgggaaaagttaggaaggacataccccaatggcagccatcgaagggatggcaagattggcaacaggtccctttctgctcccaccactttcatgaattgattatgaaagatggaagaaagttggtggtcccctgcagacctcaggatgaattaatagggagagcgagaatctctcagggagcaggatggagccttaaagaaactgcatgcctagggaaagcctacgctcaaatgtgggctctcatgtattttcacagaagagatcttagactagcatccaatgccatatgttcagcagtaccagtccattgggtccccacaagcagaacgacgtggtccatccatgctcaccatcagtggatgactacagaagacatgcttactgtttggaacagggtgtggatagaggacaatccatggatggaagacaaaactccagtcacaacttgggaagatgttccatatctagggaagagagaagaccaatggtgcggatcactcattggtctcacttccagagcaacctgggcccagaacatacttactgcaatccaacaggtaagaagccttataggcaatgaagagtttctggactacatgccttcgatgaagagattcaggaaggaggaggagtcagagggagccatttggtaaacgcaggaagcggaaaagaggcaaactgtcaggccactttaagccacagtacggaagaagctgtgcagcctgtgagccccgtccaaggacgttaaaagaagaagtcaggcccaaaagccacggtttgagcaaaccgtgctgcctgtagctccgtcgtggggacgtaaaacctgggaggctgcaaactgtggaagctgtacgcacggtgtagcagactagcggttagaggagacccctcccatgacacaacgcagcagcggggcccgagcactgagggaagctgtacctccttgcaaaggactagaggttagaggagaccccccgcaaacaaaaacagcatattgacgctgggagagaccagagatcctgctgtctcctcagcatcattccaggcacagaacgccagaaaatggaatggtgctgttgaatcaacaggttct

SEQ ID NO: 8 - DNA, 10652 bp, Dengue virus type 4
  misc_feature (7564)..(10290): Non-structural protein 5 in Dengue virus type 4, isolate D4MY01-22713, cDNA of complete genomic RNA (gi 255031234; emb FN429920.1)
agttgttagtctgtgtggaccgacaaggacagttccaaatcggaagcttgcttaacacagttctaacagtttgttttaaatagagagcagatctctggaaaaatgaaccaacgaaaaaaggtggttagaccacctttcaatatgctgaaacgcgagagaaaccgcgtatcaacccctcaagggttggtgaagagattctcaaccggacttttctccgggaaaggacccttacggatggtgctagcattcatcacgtttttgcgagtcctttccatcccgccaacagcagggattctgaaaagatggggacagttgaaaaagaataaggccatcaagatactgattggattcaggaaggagataggtcgcatgttaaacatcttgaataggagaagaaggtcaacaatgacattgctgtgtttgattcccaccgtaatggcgtttcacctgtcaacaagagacggcgaacccctcatgatagtggcaaaacacgaaagggggagacctctcttgtttaagacaacagaaggaatcaacaaatgcaccctcattgccatggacctgggtgaaatgtgtgaagacactgtcacatataaatgtcctctactggttaacaccgaacctgaagacattgattgctggtgcaatctcacgtccacctgggtcatgtacgggacatgcacccagagcggagaacggaggcgagagaagcgctcagtagctttaacaccacattcaggaatgggattggaaacaagagctgagacatggatgtcatcggaaggggcttggaaacatgctcagagagtggaaagctggatactcagaaacccaggattcgcgctcctggcaggatttatggcttacatgattgggcaaacaggaatccagcgaactgttttctttgtcctaatgatgctagtcgccccatcctacggaatgcgatgcgtaggggtagggaacagagactttgtggaaggagtctcgggtggagcatgggtcgacttggtgctagaacatggaggatgcgtcacaactatggcccagggaaaaccaaccttggattttgaactgaccaagacaacagctaaggaagtggctctgttgagaacctattgcattgaagcttcgatatcaaacataaccacggcaacaagatgtccaacgcaaggagagccttatctcaaagaagaacaagaccaacaatacatttgccggagagacgtggtagacagagggtggggtaatggctgtggcctgtttggaaaaggaggagttgtgacatgtgcgaagttttcatgctcggggaagataacaggcaatctggtccaaattgaaaaccttgaatatacagtagttgtgacagtccacaatggagacacccatgcagtaggaaatgacacatccaatcatggagtgacagccacgataactcccaggtcaccatcggtagaagttaaattgccggactatggagaactaacactcgattgtgaacctaggtccggaattgatttcaatgagatgatcctgatgaaaatgaaaaagaaaacgtggcttgtgcacaagcaatggtttttggacctacctctaccatggacagcaggagcagacacatcagaagttcattggaattataaagagagaatggtgacattcaaggttcctcatgccaagagacaggatgtgacagtgctaggatctcaggagggagctatgcattctgccctcgccggagccacagaagtggattctggtgatggaaatcacatgtttgcaggacatctcaagtgcaaagtccgtatggagaaattgagaattaagggaatgtcatatacgatgtgttcaggaaagttctcaattgacaaagagatggcagaaacacagcatgggacagcagtggtgaaagtcaagtatgaaggcgctggagctccgtgtaaaatccccatagagataagagacgtgaacaaggaaaaagtagttgggcgcatcatctcatctaccccttttgctgagaataccaacagcgtaaccaacatagaattagaacccccttttggggacagctacatagtgataggtgttggaaatagtgcattaacactccattggttcagaaaagggagctccattggcaagatgtttgagtccacatacagaggtgcaaaacgaatggccattctaggtgaaacagcttgggattttggttctgttggtggactgttcacatcactgggaaaggctgtacaccaggtttttggaagtgtgtatacaaccatgtttggaggggtctcatggataattagaatcctaattgggctcttagtattgtggattggcacgaattcaagaaacacttcaatggcaatgacgtgcatagctgttggaggaatcactctgtttctaggtttcacagttcaagcagacatgggttgtgtggtgtcatggaatgggaaggaactaaaatgtggaagcggaatttttgtggttgacaacgtgcacacttggacagaacagtacaaattccaaccagagtccccagcgaggctagcgtctgcaatattgaatgcccacaaagatggggtctgtggaatcagatcaaccacgaggctggaaaatgttatgtggaagcaaataaccaacgagctaaactatgttctctgggaaggaggacatgacctcactgtagtggctggggacgtgaagggggtgttgaccaaaggcaagagagcactcacacctccagtgaatgatctgaaatattcatggaagacatggggaaaagcaaaaatctttaccccagaagcaagaaatagcacatttttaatagacggaccagacacctccgaatgccccaatgaacgaagagcatggaacttttttgaggtagaagactatggatttggcatgttcacgaccaacatatggatgaaattccgagaaggaagttcagaagtgtgtgaccatagattaatgtcggcggcaatcaaagatcagaaagctgtgcatgctgacatgggttattggatagagagctcaaaaaaccagacctggcagatagagaaagcatcccttattgaagtgaaaacatgtctgtggcccaaaacccacacgctgtggagcaatggagtgctggaaagtcagatgctcattccaagatcatatgcaggccctttttcacagcataattaccgccagggctatgccacgcaaaccgtgggcccatggcacttaggcaaattggagatagactttggagaatgccccggaacaacagtcacaatccaggaggattgtgaccatagaggcccatctttgaggaccaccactgcatctggaaaactggtcacgcaatggtgctgccgctcctgcacgatgcctcccttaaggttcttgggagaagatggatgttggtatgggatggagatcaggcccttgagtgaaaaagaagagaacatggttaaatcacaggtaacggccggacagggtacatcagaaactttttctatggggctgctatgcctgaccttgttcgtggaagaatgcttgagaagaagagtcactaggaaacacatgatattggttgtggtaatcaccttctgtgctatcatcctgggaggtctcacatggatggacttactacgagcccttatcatgttgggggacactatgtctggtagaataggaggacagattcacctagccatcatggcagtgttcaagatgtcaccaggatatgtgctgggtgtgtttttaaggaaacttacttcaagagagacagcactaatggtaataggaatggccatgacaacggtgttttcaattccacatgacctcatggaactcattgatggaatatcgctggggttgatattgttaaaaatagtaacacattttgataacacccaagtgggaaccttagccctttccctgactttcataagatcaacaacaccattagtcatggcttggaggaccattatggctgtgttctttgtggtcacactcattcctttgtgtaggacaagctgtcttcaaaaacagtcccattgggtagaaataacagcacttatcttaggagcccaggctttgccagtgtacctaatgaccctcatgaaaggagcctcaagaagatcttggcctcttaatgagggcataatggctgtgggtttggtgagcctcttaggaagcgccctcctaaagaatgatgttcctttagctggcccaatggtggcaggaggcttacttctagcggcttacgtaatgagtggtagctcggcagacttgtcactagagaaggctgccaatgtgcagtgggatgaaatggcggacataactggctcaagcccaatcatagaagtaaagcaggatgaagatggctctttctccatacgggacgtcgaggaaaccaacatgataaccctcttggtgaaactggcactgataacagtatcaggtctttaccccttggcaattccagtcacaatggcattatggtatatttggcaagtgaaaacacaaagatcaggagctctgtgggacgtcccctcacccgctgccactcagaaagccacactgtctgaaggagtgtataggatcatgcaaagagggttgtttgggaaaactcaggttggagtagggatacacatggaaggtgtattccacacaatgtggcacgtgacaagaggatcagtgatctgccatgagacagggagattagagccatcttgggctgacgtcaggaatgacatgatatcatacggtgggggatggaggctcggagacaaatgggacaaagaagaagatgttcaggtcctagccatagaaccaggaaaaaatcctaaacatgtccaaacgaaaccaggccttttcaagaccctaactggagaaattggagcagtaactctggatttcaaacccggaacgtctggctctcctatcattaacaagaaagggaaagttattggactctatggaaatggagtagttaccaaatcaggtgattacgtcagtgccataacgcaagctgaaagaattggtgagccagattatgaagtggatgaggacatttttcgaaagaaaagattaaccataatggacttacaccccggagctggaaagacaaaaagaattctcccatcaatagtcagagaagctttaaaaaggaggctgcgaactttgattttggctcctacgagagtggtggcagccgagatggaagaggccctacgcggactgccaatccgttatcagaccccagctgtgaaatcagatcacacaggaagagagattgtggacctcatgtgtcatgcaaccttcacaacgagacttttgtcatcaaccagggttccaaattataacctcatagtgatggatgaagcacatttcactgacccttgtagtgtcgcggctagaggatacatttcaaccagggtggaaatgggagaggcagcagccatcttcatgactgcaacccctcctggatcgatagatcccttcccccagagcaacagcccgatagaagacatcgaaagggaaatcccagaaaggtcatggaacacagggttcgactggataacagactaccaagggaaaactgtgtggtttgttcccagcataaaagctggaaatgacattgcaaattgtttgcgaaagtcgggaaagagagtgatccagttgagcaggaaaacctttgacacagagtatccaaaaacgaaactcacggactgggattttgtggtaaccacagacatatctgaaatgggggccaatttcagagctgggagagtgatagaccccaggagatgcctcaagccagttatcctaacagatgggccagagagagttattctagcaggtccaatcccagtaactccagcaagtgccgctcagagaagagggcgaataggtaggaatccagcacaagaagatgaccaatacgttttctccggagacccactaaagaatgatgaagatcatgcccactggacagaagcgaagatgttgcttgacaatatctacacccctgaagggataattccaacattgtttggtccggaaagggaaaaaacccaagccattgatggagagtttcgcctcagaggggaacaaaggaagacttttgtggaattaatgaggagaggagaccttccggtgtggctgagctacaaggtagcttctgccggtatctcttacaaagaccgagaatggtgcttcacaggggaaaggaacaaccaaattttagaagaaaacatggaggttgaaatctggactagagagggagaaaagaaaaaactaaggccaaaatggttagatgcacgtgtgtacgctgaccccatggctttgaaggatttcaaggagtttgccagtggaagaaagagcataactcttgacatcctaacagagattgccaccttgccaacttacctctcctctaaggccaagctagcccttgacaacatagtcatgctccacacaacagaaaaaggagggagggcctaccaacacgccttgaacgaactcccggaatcactggaaacactcatgcttgtagccctactaggtgctatgacagccggtatcttcctgtttttcatgcaggggaaaggaatagggaaattgtcaatgggtctgatagccatagctgtggctagtggtttgctttgggtagcagaaattcagccccagtggatagcagcttcaatcatactggagttttttcttatggtgctgttgataccagaaccagaaaaacaaaggaccccacaagacaatcaattgatctacgtcatattgaccattctcaccattattggtcttatagcagccaatgagatggggctgattgaaaaaacaaaaacggactttgggttttaccaggtaaaaacagaaaccaccattcttgatgtggatttgagaccagcctcagcatggacactctatgcagtggccaccactattctgactcccatgctgagacacaccatagagaacacgtctgccaacctatctctagcagccattgccaaccaagcagctgtcttaatgggacttggaaaaggatggccgctccacagaatggacctcggtgtgccgctgttagcaatgggatgttattctcaagtaaacccaacaaccctgacagcatccttagtcatgcttttagtccattatgccataataggtccaggattgcaggcaaaagccacaagagaggcccagaaaaggacagctgctgggatcatgaagaaccccacggtggacgggataacagtaatagatctagaaccaatatcttatgacccaaaatttgaaaaacagttagggcaagtcatgctactggtcctgtgtgttggacaattacttttgatgagaacaacatgggctctctgcgaagtattgactttggccacaggaccaatcatgaccttgtgggagggcaacccgggaaggttttggaacacgaccatagctgtttccacagccaacatttttaggggaagctacttggcgggagctggactagctttttcactcataaagaatgtacaaacccctaggaggggaactgggaccacaggagagacactgggagagaagtggaagagacagctgaactcattagatagaaaagagtttgaagagtacaaaagaagtggaatactagaagtggataggactgaagccaagtctgccctaagagatggatctaaaatcaagcatgcggtgtccagagggtctagtaagatcagatggattgttgaaagagggatgataaagccaaaaggaaaggtcgtggatcttggttgtgggaggggaggatggtcctattacatggcgacactcaagaacgtgactgaagtgaaaggatatacaaaaggaggtccaggacatgaagaaccaatccccatggctacttatggctggaatttggtcaaactccattcaggggttgatgtgttctacaaacccactgagcaggtggacaccctgctttgtgacattggggagtcatcctctaatccgacaatagaggaaggaagaacattgagagtcttgaagatggtggagccatggctctcttcaaaacctgagttctgcatcaaagtcctcaacccctacatgccaacagttatagaagagctggagaagctgcagagaagacatggtggaagcctcgtcagatgcccgctatccaggaattctactcatgagatgtattgggtgtcaggtgcgtcgggaaacatcgtgagttctgtgaacacaatatcaaagatgctgttgaacaggttcacaacaaggcataggaaacccacttatgagaaggacgtagatcttggggcaggaacgagaagtgtctccactgaaacagaaaaaccagacatgacaattattgggagaaggcttcagagactgcgagaagagcacaaagaaacttggcactatgaccaggaaaacccatacagaacctgggcgtatcatggaagctatgaagctccttcgacaggctcagcatcctccatggtgaacggggtagtaaaactgctgacaaaaccttgggatgtgattccaatggtgacccagttggccatgacagacacaaccccttttgggcaacaaagagtgttcaaggagaaggtggataccagaacaccacaaccaaaacccggcacacgaatgattatgaccacgacagccaattggctgtgggccctcctcgggaagaagaaaaatcccagactgtgcacaagggaagagttcatctcaaaagttagatcaaatgcagccataggcgcagtctttcaggaagaacagggatggacatcagccagtgaagctgtgaatgacagccggttctgggaactggttgacaaagaaagggctctgcaccaggaaggaaaatgtgaatcgtgtgtctataacatgatgggaaaacgtgagaaaaaattaggagagtttggtagggccaagggaagccgagcaatctggtacatgtggctgggggcgcggttcctggaatttgaagccctgggttttttgaatgaagatcactggtttagcagagaaaactcatggagtggagtggaaggggaaggtctgcatagattgggatacatcttggaggacatagacaagaaggatggagatctaatatatgctgacgacacagcaggctgggacacgagaatcactgaggatgaccttttaaatgaagaactgatcacagaacagatggcccctcaccacaagaccctagccaaggccattttcaaactaacctatcaaaacaaagtggtgaaagtcctcagacccacaccgaaaggagcggtaatggacattatatccaggaaagaccaaagaggtagtggacaggtgggaacatatggtttaaacacattcactaacatggaagttcaactcatccgccaaatggaagctgaaggagtcatcacacaagacgacatgcagaacccaaaagggttgaaagaaagagttgagaaatggctgaaagagtgtggcgtcgacaggttgaagaggatggcaatcagtggagatgattgcgtggtgaagcccctagatgagaggtttagcacctccctcctcttcttgaatgacatgggaaaggtgaggaaagacatcccgcagtgggaaccatccaagggatggaaaaactggcaagaggttcctttttgctcccaccattttcacaagatcttcatgaaagatggccgctcactagttgttccatgcagaaaccaggatgaactgataggaagagccagaatctcgcagggggctggatggagcttaagggaaacagcctgtctgggcaaagcttacgcccagatgtggtcgcttatgtacttccatagaagggacctgcgtttagcctccatggccatatgctcagcagttccaacggaatggtttccaacaagcagaacaacatggtcgatccacgctcatcaccagtggatgaccactgaagacatgcttaaagtgtggaacagagtgtggatagaagacaaccccaatatgactgacaagactccagtccattcgtgggaagacataccttacctagggaaaagagaggatttgtggtgtggatccctgattggactttcttccagagccacctgggcgaagaacattcacacggccataacccaggtcaggaatctgatcggaaaagaggaatacgtggattacatgccagtcatgaaaagatacagcgctccctccgagagtgaaggagttctgtaattatcaacaacaaacaccaaagagaccattgaagtcaggccacttgtgccacggcttgagcaaaccgtgctgcctgtagctccgccaataatgggaggcgtaaaattcccagggaggccatgcgccacggaagctgtacgcgtggcatattggactagcggttagaggagacccctcccatcactgacaaaacgcagcaaaaagggggcccgaagccaggaggaagctgtacttctggtggaaggactagaggttagaggagacccccccaacacaaaaacagcatattgacgctgggaaagaccagagatcctgctgtctctgcaacatcaatccaggcacagagcgccgcgagatggattggtgttgttgatccaacaggttct

SEQ ID NO: 9 - protein, 3389 aa, Dengue virus type 1
  MISC_FEATURE (2494)..(3389): Non-structural protein 5 in DENV-1 Westpac74 MT mutant passage 10
MNNQRKKTGRPSFNMLKRARNRVSTVSQLAKRFSKGLLSGQGPMKLVMAFIAFLRFLAIPPTAGILARWGSFKKNGAIKVLRGFKKEISNMLNIMNRRKRSVTMLLMLLPTALAFHLTTRGGEPHMIVSKQERGKSLLFKTSAGVNMCTLIAMDLGELCEDTMTYKCPRITETEPDDVDCWCNATETWVTYGTCSQTGEHRRDKRSVALAPHVGLGLETRTETWMSSEGAWKQIQKVETWALRHPGFTVIALFLAHAIGTSITQKGIIFILLMLVTPSMAMRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKTIHEWCCRSCTLPPLRFKGEDGCWYGMEIRPVKEKEENLVKSMVSAGSGEVDSFSLGLLCISIMIEEVMRSRWSRKMLMTGTLAVFLLLTMGQLTWNDLIRLCIMVGANASDKMGMGTTYLALMATFRMRPMFAVGLLFRRLTSREVLLLTVGLSLVASVELPNSLEELGDGLAMGIMMLKLLTDFQSHQLWATLLSLTFVKTTFSLHYAWKTMAMILSIVSLFPLCLSTTSQKTTWLPVLLGSLGCKPLTMFLITENKIWGRKSWPLNEGIMAVGIVSILLSSLLKNDVPLAGPLIAGGMLIACYVISGSSADLSLEKAAEVSWEEEAEHSGASHNILVEVQDDGTMKIKDEERDDTLTILLKATLLAISGVYPMSIPATLFVWYFWQKKKQRSGVLWDTPSPPEVERAVLDDGIYRILQRGLLGRSQVGVGVFQEGVFHTMWHVTRGAVLMYQGKRLEPSWASVKKDLISYGGGWRFQGSWNAGEEVQVIAVEPGKNPKNVQTAPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIKRKLRTLVLAPTRVVASEMAEALKGMPIRYQTTAVKSEHTGKEIVDLMCHATFTMRLLSPVRVPNYNMIIMDEAHFTDPASIAARGYISTRVGMGEAAAIFMTATPPGSVEAFPQSNAVIQDEERDIPERSWNSGYDWITDFPGKTVWFVPSIKSGNDIANCLRKNGKRVVQLSRKTFDTEYQKTKNNDWDYVVTTDISEMGANFRADRVIDPRRCLKPVILKDGPERVILAGPMPVTVASAAQRRGRIGRNQNKEGDQYIYMGQPLKNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEIWTKEGERKKLRPRWLDARTYSDPLALREFKEFAAGRRSVSGDLILEIGKLPQHLTQRAQNALDNLVMLHNSEQGGKAYRHAMEELPDTIETLMLLALIAVLTGGVTLFFLSGRGLGKTSIGLLCVIASSALLWMASVEPHWIAASIILEFFLMVLLIPEPDRQRTPQDNQLAYVVIGLLFMILTVAANEMGLLETTKKDLGIGHAAAENHHHAAMLDVDLHPASAWTLYAVATTIITPMMRHTIENTTANISLTAIANQAAILMGLDKGWPISKMDIGVPLLALGCYSQVNPLTLTAAVFMLVAHYAIIGPGLQAKATREAQKRTAAGIMKNPTVDGIVAIDLDPVVYDAKFEKQLGQIMLLILCTSQILLMRTTWALCESITLATGPLTTLWEGSPGKFWNTTIAVSMANIFRGSYLAGAGLAFSLMKSLGGGRRGTGAQGETLGEKWKRQLNQLSKSEFNTYKRSGIIEVDRSEAKEGLKRGETTKHAVSRGTAALRWFVERNLVKPEGKVIDLGCGRGGWSYYCAGLKKVTEVKGYTKGGPGHEEPIPMATYGWNLVKLYSGKDVFFTPPEKCDTLLCDIGESSPNPTIEEGRTLRVLKMVEPWLRGNQFCIKILNPYMPSVVETLEQMQRKHGGMLVRNPLSRNSTHAMYWVSCGTGNIVSAVNMTSRMLLNRFTMAHRKPTYERDVDLGAGTRHVAVEPEVANLDIIGQRIENIKNEHKSTWHYDEDNPYKTWAYHGSYEVKPSGSASSMVNGVVRLLTKPWDVIPMVTQIAMTDTTPFGQQRVFKEKVDTRTPKAKRGTAQIMEVTARWLWGFLSRNKKPRICTREEFTRKVRSNAAIGAVFVDENQWNSAKEAVEDERFWDLVHRERELHKQGKCATCVYNMMGKREKKLGEFGKAKGSRAIWYMWLGARFLEFEALGFMNEDHWFSRENSLSGVEGEGLHKLGYILRDISKIPGGNMYADDTAGWDTRITEDDLQNEAKITDIMEPEHALLATSIFKLTYQNKVVRVQRPAKNGTVMDVISRRDQRGSGQVGTYGLNTFTNMEAQLIRQMESEGIFSPSELETPNLAERVLDWLKKHGTERLKRMAISGDDCVVKPIDDRFATALTALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEDVPYLGKREDQWCGSLIGLTARATWATNIQVAINQVRRLIGNENYLTSMKRFKNESDPEGALW

SEQ ID NO: 10 - protein, 3391 aa, Dengue virus type 2
  MISC_FEATURE (2492)..(3391): Non-structural protein 5 in DENV-2 MT mutant passage 10
MNNQRKKARNTPFNMLKRERNRVSTVQQLTKRFSLGMLQGRGPLKLFMALVAFLRFLTIPPTAGILKRWGTIKKSKAINVLRGFRKEIGRMLNILNRRRRTAGIIIMMIPTVMAFHLTTRNGEPHMIVSRQEKGKSLLFKTENGVNMCTLMAMDLGELCEDTITYNCPLLRQNEPEDIDCWCNSTSTWVTYGTCTATGEHRREKRSVALVPHVGMGLETRTETWMSSEGAWKHAQRIETWVLRHPGFTIMAAILAYTIGTTYFQRVLIFILLTAVTPSMTMRCIGISNRDFVEGVSGGSWVDIVLEHGSCVTTMAKNKPTLDFELVKTEAKHPATLRKYCIEAKLTNTTTASRCPTQGEPSLNEEQDKRFVCKHSMVDRGWGNGCGLFGKGGIVTCAMFTCKKNMEGKVVQPENLEYTIVITPHSGEENAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLSWFKKGSSIGQMFETTMRGAKRMAILGDTAWDFGSLGGVFTSIGKALHQVFGAIYGAAFSGVSWTMKILIGVVITWIGMNSRSTSLSVSLVLVGVVTLYLGVMVQADSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKAWGKAKMLSTELHNHTFLIDGPETAECPNTNRAWNSLEVEDYGFGVFTTNIWLKLKERQDVFCDSKLMSAAIKDNRAVHADMGYWIESALNDTWKIEKASFIEVKSCHWPKSHTLWSNGVLESEMIIPKNFAGPVSQHNYRPGYHTQTAGPWHLGRLEMDFDFCEGTTVVVTEDCGNRGPSLRTTTASGKLITEWCCRSCTLPPLRYRGEDGCWYGMEIRPLKEKEENLVNSLVTAGHGQIDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKIVRNMEKYQLAVTIMAILCVPNAVILQNAWKVSCTTLAVVSVSPLLLTSSQQKADWIPLALTIKGLNPTAIFLTTLSRTSKKRSWPLNEAIMAVGMVSILASSLLKNDIPMTGPLVAGGLLTVCYVLTGRSADLELERAADVRWEEQAEISGSSPILSITISEDGSMSIKNEEEEQTLTILIRTGLLVISGLFPASIPITAAAWYLWEVKKQRAGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNTGTIGAVSLDFSPGTSGSPIVDKKGKVVGLYGNGVVTRSGAYVSAIAQTEKSIEDNPEIEDDIFRKKRLTIMDLHPGAGKTKRYLPAIVREAIKRGLRTLILAPTRVVAAEMEEALRGLPIRYQTPAIRAEHTGREIVDLMCHATFTMRLLSPIRVPNYNLIIMDEAHFTDPASIAARGYISTRVEMGEAAGIFMTATPPGSRDPFPQSNAPIMDEEREIPERSWNSGHEWVTDFKGKTVWFVPSIKAGNDIAACLRKNGKKVIQLSRKTFDSEYIKTRTNDWDFVVTTDISEMGANFKAERVIDPRRCMKPVILTDGEERVILAGPMPVTHSSAAQRRGRVGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARNALDNLAVLHTAEAGGRAYNHALSELPETLETLLLLTLLATVTGGIFLFLMSGKGIGKMTLGMCCIITASILLWYAQIQPHWIAASIILEFFLIVLLIPEPEKQRTPQDNQLTYVVIAILTVVAATMANEMGFLEKTKKDFGLGSIATQQPESNILDIDLRPASAWTLYAVATTFITPMLRHSIENSSVNVSLTAIANQATVLMGLGKGWPLSKMDIGVPLLAIGCYSQVNPITLTAALLLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTANTRRGTGNTGETLGEKWKNRLNALGKSEFQIYKKSGIQEVDRTLAKEGIKRGETDHHAVSRGSAALRWFVERNLVTPEGKVVDLGCGRGGWSYYCGGLKNVKEVKGLTKGGPGHEEPIPMSTYGWNLVRLQSGVDVFFTPPEKCDTLLCDIGESSPNPTVEAGRTLRVLNLVENWLNNNTQFCIKVLNPYMPSVIEKMEALQRKYGGALVRNPLSRNSTHAMYWVSNASGNIVSSVNMISRMLINRFTMRHKKATYEPDVDLGSGTRNIGIESETPNLDIIGKRIEKIKQEHETSWHYDQDHPYKTWAYHGSYETKQTGSASSMVNGVVRLLTKPWDIIPMVTQMAMTDTTPFGQQRVFKEKVDTRTQEPKEGTKKLMKITAEWLWKELGKKKTPRMCTREEFTRKVRSNAALGAIFTDENKWKSAREAVEDSGFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVVTYGLNTFTNMEAQLIRQMEGEGVFKSIQHLTVTEEIAVKNWLVRVGRERLSRMAISGDDCVVKPLDDRFASALTALNDMGKVRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHATHEWMTTEDMLTVWNRVWIQENPWMEDKTPVESWEEIPYLGKREDQWCGSLIGLTSRATWAKNIQTAINQVRSLIGNEEYTDYMPSMKRFRREEEEAGVLW

SEQ ID NO: 11 - protein, 3390 aa, Dengue virus type 3
  MISC_FEATURE (2491)..(3390): Non-structural protein 5 in DENV-3 MT mutant passage 5
MNNQRKKTGKPSINMLKRVRNRVSTGSQLAKRFSRGLLNGQGPMKLVMAFIAFLRFLAIPPTAGILARWGTFKKSGAIKVLRGFKKEISNMLSIINRRKKTSLCLMMMLPATLAFHLTSRDGEPRMIVGKNERGKSLLFKTASGINMCTLIAMDLGEMCDDTVTYKCPLITEVEPEDIDCWCNLTSTWVTYGTCNQAGEHRRDKRSVALAPHVGMGLDTRAQTWMSAEGAWRQVEKVETWAFRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNVTTDSRCPTQGEAILPEEQDQNYVCKHTYVDRGWGNGCGLFGKGSLVTCAKFQCLELIEGKVVQHENLKYTVIITVHTGDQHQVGNETQGVTAEITPQASTVEAILPEYGTLGLECSPRTGLDFNEMILLTMKNKAWMVHRQWFFDLPLPWTSGATTETPTWNKKELLVTFKNAHAKKQEVVVLGSQEGAMHTALTGATEIQTSGGTSIFAGHLKCRLKMDKLELKGMSYAMCSNAFVLKKEVSETQHGTILIKVEYKGEDAPCKIPFSTEDGQGKAHNGRLITANPVVTKKEEPVNIEAEPPFGESNIIIGTGDKALKINWYKKGSSIGKMFEATARGARRMAILGDTAWDFGSVGGVLNSLGKMVHQIFGSAYTALFSGVSWIMKIGIGVLLTWIGLNSKNTSMSFSCIVIGIITLYLGAVVQADMGCVINWKGKELKCGSGIFVTNEVHTWTEQYKFQADSPKRLATAIAGAWENGVCGIRSTTRMENLLWKQIANELNYILWENNIKLTVVVGDIIGILEQGKRTLTPQPMELKYSWKTWGKAKIVTAEIQNSSFIIDGPNTPECPNASRAWNVWEVEDYGFGVFTTNIWLKLREMYTQLCDHRLMSAAVKDERAVHADMGYWIESQKNGSWKLEKASLIEVKTCTWPKSHTLWSNGVLESDMIIPKSLAGPISQHNYRPGYHTQTAGPWHLGKLELDFNYCEGTTVVITENCGTRGPSLRTTTVSGKLIHEWCCRSCTLPPLRYMGEDGCWYGMEIRPINEKEENMVKSLVSAGSGKVDNFTMGVLCLAILFEEVMRGKFGKKHMIAGVLFTFVLLLSGQITWRDMARTLIMIGSNASDRMGMGVTYLALIATFKIQPFLALGFFLRKLTSRENLLLGVGLAMATTLQLPEDIEQMANGIALGLMALKLITQFETYQLWTALVSLMCSNTIFTLTVAWRTATLILAGISLLPVCQSSSMRKTDWLPMTVAAMGVPPLPLFIFSLKDTLKRRSWPLNEGVMAVGLVSILASSLLRNDVPMAGPLVAGGLLIACYVITGTSADLTVEKAADVTWEEEAEQTGVSHNLMITVDDDGTMRIKDDETENILTVLLKTALLIVSGIFPYSIPATLLVWHTWQKQTQRSGVLWDVPSPPETQKAELEEGVYRIKQQGIFGKTQVGVGVQKEGVFHTMWHVTRGAVLTYNGKRLEPNWASVKKDLISYGGGWRLSAQWQKGEEVQVIAVEPGKNPKNFQTMPGIFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGKEIVDLMCHATFTMRLLSPVRVPNYNLIIMDEAHFTDPASIAARGYISTRVGMGEAAAIFMTATPPGTADAFPQSNAPIQDEERDIPERSWNSGNDWITDFAGKTVWFVPSIKAGNDIANCLRKNGKKVIQLSRKTFDTEYQKTKLNDWDFVVTTDISEMGANFKADRVIDPRRCLKPVILTDGPERVILAGPMPVTVASAAQRRGRVGRNPQKENDQYIFTGQPLNNDEDHAHWTEAKMLLDNINTPEGIIPALFEPEREKSAAIDGEYRLKGESRKTFVELMRRGDLPVWLAHKVASEGIKYTDRKWCFDGERNNQILEENMDVEIWTKEGEKKKLRPRWLDARTYSDPLALKEFKDFAAGRKSIALDLVTEIGRVPSHLAHRTRNALDNLVMLHTSEHGGRAYRHAVEELPETMETLLLLGLMILLTGGAMLFLISGKGVGKTSIGLICVVASSGMLWMADIPLQWIASAIVLEFFMMVLLIPEPEKQRTPQDNQLAYVVIGILTLAAIVAANEMGLLETTKRDLGMSKEPGVASPTSYLDVDLHPASAWTLYAVATTVITPMLRHTIENSTANVSLAAIANQAVVLMGLDKGWPISKMDLGVPLLALGCYSQVNPLTLTAAVLLLVTHYAIIGPGLQAKATREAQKRTAAGIMKNPTVDGIMTIDLDPVIYDSKFEKQLGQVMLLVLCAVQLLLMRTSWAFCEALTLATGPITTLWEGSPGKFWNTTIAVSMANIFRGSYLAGAGLAFSIMKSVGTGKRGTGSQGETLGEKWKKKLNQLSWKEFDLYKKSGITEVDRIEAKEGLKRGEITHHAVSRGSAALQWFVERNMVIPEGRVIDLGCGRGGWSYYCAGLKKVTEVRGYTKGGPGHEEPVPMSTYGWNIVKLMSGKDVFYLPPEKCDTLLCDIGESSPSPTVEESRTIRVLKMVEPWLKNNQFCIKVLNPYMPAVIEHLERLQRKHGGMLVRNPLSRNSTHAMYWISNGTGNIVSSVNMVSRLLLNRFTMTYRKPTIEKDVDLGAGTRHVNAEPETPNMDVIGERIRRIKEEHSSTWHYDDENPYKTWAYHGSYEVKATGSASSMINGVVKLLTKPWDVVPTVTQMAMTDTTPFGQQRVFKEKVDTRTPKPMPGTRKVMEITAEWLWRTLGRNKRPRLCTREEFTKKVRTNAAMGAVFTEENQWDSARAAVEDEEFWKLVDRERELHKLGKCGSCVYNMMGKREKKLGEFGKAKGSRAIWYMWLGARYLEFEALGFLNEDHWFSRENSYSGVEGEGLHKLGYILRDISKIPGGAMYADDTAGWDTRITEDDLHNEEKITQQMDPEHRQLANAIFKLTYQNKVVKVQRPTPKGTVMDIISRKDQRGSGQVGTYGLNTFTNMEAQLIRQMEGEGVLSKTDLENPHLLEKKITQWLETKGVERLKRMAISGDDCVVKPIDDRFANALLALNDMGKVRKDIPQWQPSKGWQDWQQVPFCSHHFHELIMKDGRKLVVPCRPQDELIGRARISQGAGWSLKETACLGKAYAQMWALMYFHRRDLRLASNAICSAVPVHWVPTSRTTWSIHAHHQWMTTEDMLTVWNRVWIEDNPWMEDKTPVTTWEDVPYLGKREDQWCGSLIGLTSRATWAQNILTAIQQVRSLIGNEEFLDYMPSMKRFRKEEESEGAIW

SEQ ID NO: 12 - protein, 3387 aa, Dengue virus type 4
  MISC_FEATURE (2488)..(3387): Non-structural protein 5 of DENV-4 MT mutant passage 5
MNQRKKVVRPPFNMLKRERNRVSTPQGLVKRFSTGLFSGKGPLRMVLAFITFLRVLSIPPTAGILKRWGQLKKNKAIKILIGFRKEIGRMLNILNRRRRSTMTLLCLIPTVMAFHLSTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYGMRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTAVVKVKYEGAGAPCKIPIEIRDVNKEKVVGRIISSTPFAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWIIRILIGLLVLWIGTNSRNTSMAMTCIAVGGITLFLGFTVQADMGCVVSWNGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVNDLKYSWKTWGKAKIFTPEARNSTFLIDGPDTSECPNERRAWNFFEVEDYGFGMFTTNIWMKFREGSSEVCDHRLMSAAIKDQKAVHADMGYWIESSKNQTWQIEKASLIEVKTCLWPKTHTLWSNGVLESQMLIPRSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTAGQGTSETFSMGLLCLTLFVEECLRRRVTRKHMILVVVITFCAIILGGLTWMDLLRALIMLGDTMSGRIGGQIHLAIMAVFKMSPGYVLGVFLRKLTSRETALMVIGMAMTTVFSIPHDLMELIDGISLGLILLKIVTHFDNTQVGTLALSLTFIRSTTPLVMAWRTIMAVFFVVTLIPLCRTSCLQKQSHWVEITALILGAQALPVYLMTLMKGASRRSWPLNEGIMAVGLVSLLGSALLKNDVPLAGPMVAGGLLLAAYVMSGSSADLSLEKAANVQWDEMADITGSSPIIEVKQDEDGSFSIRDVEETNMITLLVKLALITVSGLYPLAIPVTMALWYIWQVKTQRSGALWDVPSPAATQKATLSEGVYRIMQRGLFGKTQVGVGIHMEGVFHTMWHVTRGSVICHETGRLEPSWADVRNDMISYGGGWRLGDKWDKEEDVQVLAIEPGKNPKHVQTKPGLFKTLTGEIGAVTLDFKPGTSGSPIINKKGKVIGLYGNGVVTKSGDYVSAITQAERIGEPDYEVDEDIFRKKRLTIMDLHPGAGKTKRILPSIVREALKRRLRTLILAPTRVVAAEMEEALRGLPIRYQTPAVKSDHTGREIVDLMCHATFTTRLLSSTRVPNYNLIVMDEAHFTDPCSVAARGYISTRVEMGEAAAIFMTATPPGSIDPFPQSNSPIEDIEREIPERSWNTGFDWITDYQGKTVWFVPSIKAGNDIANCLRKSGKRVIQLSRKTFDTEYPKTKLTDWDFVVTTDISEMGANFRAGRVIDPRRCLKPVILTDGPERVILAGPIPVTPASAAQRRGRIGRNPAQEDDQYVFSGDPLKNDEDHAHWTEAKMLLDNIYTPEGIIPTLFGPEREKTQAIDGEFRLRGEQRKTFVELMRRGDLPVWLSYKVASAGISYKDREWCFTGERNNQILEENMEVEIWTREGEKKKLRPKWLDARVYADPMALKDFKEFASGRKSITLDILTEIATLPTYLSSKAKLALDNIVMLHTTEKGGRAYQHALNELPESLETLMLVALLGAMTAGIFLFFMQGKGIGKLSMGLIAIAVASGLLWVAEIQPQWIAASIILEFFLMVLLIPEPEKQRTPQDNQLIYVILTILTIIGLIAANEMGLIEKTKTDFGFYQVKTETTILDVDLRPASAWTLYAVATTILTPMLRHTIENTSANLSLAAIANQAAVLMGLGKGWPLHRMDLGVPLLAMGCYSQVNPTTLTASLVMLLVHYAIIGPGLQAKATREAQKRTAAGIMKNPTVDGITVIDLEPISYDPKFEKQLGQVMLLVLCVGQLLLMRTTWALCEVLTLATGPIMTLWEGNPGRFWNTTIAVSTANIFRGSYLAGAGLAFSLIKNVQTPRRGTGTTGETLGEKWKRQLNSLDRKEFEEYKRSGILEVDRTEAKSALRDGSKIKHAVSRGSSAIRWIVERGMIKPKGKVVDLGCGRGGWSYYMATLKNVTEVKGYTKGGPGHEEPIPMATYGWNLVKLHSGVDVFYKPTEQVDTLLCDIGESSSNPTIEEGRTLRVLKMVEPWLSSKPEFCIKVLNPYMPTVIEELEKLQRRHGGSLVRCPLSRNSTHAMYWVSGASGNIVSSVNTISKMLLNRFTTRHRKPTYEKDVDLGAGTRSVSTETEKPDMTIIGRRLQRLREEHKETWHYDQENPYRTWAYHGSYEAPSTGSASSMVNGVVKLLTKPWDVIPMVTQLAMTDTTPFGQQRVFKEKVDTRTPQPKPGTRMIMTTTANWLWALLGKKKNPRLCTREEFISKVRSNAAIGAVFQEEQGWTSASEAVNDSRFWELVDKERALHQEGKCESCVYNMMGKREKKLGEFGRAKGSRAIWYMWLGARFLEFEALGFLNEDHWFSRENSWSGVEGEGLHRLGYILEDIDKKDGDLIYADDTAGWDTRITEDDLLNEELITEQMAPHHKTLAKAIFKLTYQNKVVKVLRPTPKGAVMDIISRKDQRGSGQVGTYGLNTFTNMEVQLIRQMEAEGVITQDDMQNPKGLKERVEKWLKECGVDRLKRMAISGDDCVVKPLDERFSTSLLFLNDMGKVRKDIPQWEPSKGWKNWQEVPFCSHHFHKIFMKDGRSLVVPCRNQDELIGRARISQGAGWSLRETACLGKAYAQMWSLMYFHRRDLRLASMAICSAVPTEWFPTSRTTWSIHAHHQWMTTEDMLKVWNRVWIEDNPNMTDKTPVHSWEDIPYLGKREDLWCGSLIGLSSRATWAKNIHTAITQVRNLIGKEEYVDYMPVMKRYSAPSESEGVL

(19) United States
(12) Patent Application Publication
(10) Pub. No.: US 2015/0231226 A1
(43) Pub. Date: Aug. 20, 2015

(54) NOVEL ATTENUATED DENGUE VIRUS STRAINS FOR VACCINE APPLICATION

(71) Applicants: Agency for Science, Technology and Research, Singapore (SG); Novartis AG, Basel (CH); Beijing Institute of Microbiology and Epidemiology, Beijing (CN)

(72) Inventors: Katja Fink, Singapore (SG); Pei-Yong Shi, Singapore (SG); Cheng Feng Qin, Singapore (SG)

(73) Assignees: Agency for Science, Technology and Research, Singapore (SG); Novartis AG, Basel (CH); Beijing Institute of Microbiology and Epidemiology, Beijing (CN)

(21) Appl. No.: 14/430,099

(22) PCT Filed: Sep. 23, 2013
(86) PCT No.: PCT/SG2013/000413

§ 371 Date: Mar. 20, 2015

(30) Foreign Application Priority Data
Sep. 21, 2012 (SG) 201207042-1

(51) Int. Cl. A61K 39/12; C12N 7/00

(52) U.S. Cl. CPC A61K 39/12; C12N 7/00; C12N 2770/24134; C12N 2770/24121; A61K 2039/5252

(57) ABSTRACT

The present invention discloses a method of eliciting an immune response and a method of vaccination comprising administration of a mutated flavivirus. The mutated flavivirus comprises at least one mutation in a nucleic acid sequence encoding for the non-structural protein 5 of the flavivirus sequence resulting in inactivation of the 2′O-methyltransferase.

### CROSS-REFERENCE TO RELATED APPLICATIONS

This application claims the benefit of priority of SG provisional application No. 201207042-1, filed Sep. 21, 2012, the contents of it being hereby incorporated by reference in its entirety for all purposes.

### FIELD OF THE INVENTION

The invention relates to the field of immunology and virology, and to mutated viruses, vaccines, pharmaceutical compositions and related methods.

### BACKGROUND OF THE INVENTION

Flavivirus is a genus of the family Flaviviridae. This genus includes the dengue virus (DENV), tick borne encephalitis virus (TBEV), West Nile virus (WNV), and several other viruses, which may cause encephalitis. Flaviviruses are positive-sense, single-stranded RNA viruses. The flaviviruses' genome encodes for three structural (C, prM, and E), and seven non-structural proteins (NS1, NS2A, NS2B, NS3, NS4A, NS4B, and NS5), the latter being the largest and most highly conserved of the dengue proteins. NS5 is a multifunctional protein, and its N-terminus is the S-adenosyl-L-methionine dependent methyltransferase (SAM) domain (amino acids 1-320), which possesses the methyltransferase (MTase) and guanylyl transferase activity responsible for capping and methylating the capped the positive strand genomic RNA on its 5′ terminus.

Dengue virus (DENV) causes dengue fever (DF) and more severe forms of the disease, namely dengue hemorrhagic fever (DHF) and dengue shock syndrome (DSS). DENV includes four serotypes (DENV1-4), each of which is capable of causing severe disease. Over the past decade, cases have increased in frequency, severity and geographical spread. Every year one hundred million new cases of dengue fever and 250.000 dengue hemorrhagic fever/dengue shock syndrome are estimated. At present, despite worldwide intensive research efforts, no vaccine or cure for dengue infection is available. Vaccine development is complex because of multiple factors: i) an effective vaccine must consist of a tetravalent formulation protecting against each of the four serotypes because multiple serotypes typically circulate in a geographical region, and ii) a sub-protective vaccine potentially increases the risk of vaccinated individuals to become more susceptible to the more severe forms of dengue disease during repeated infection because of a known association of pre-existing immunity with severity. Since most infections occur in developing countries, an ideal vaccine should be affordable as well as highly protective. This requires a highly immunogenic vaccine, inducing a robust level of immunity, ideally with only one inoculation.

Due to the limitations of current vaccine candidates in clinical testing, development of “second generation” vaccines is needed.

Thus, an object of the invention is to ameliorate at least one of the above-mentioned problems.

### SUMMARY OF THE INVENTION

Accordingly in a first aspect of the invention, there is provided a method of eliciting an immune response comprising administration of a mutated flavivirus comprising at least one mutation in a nucleic acid sequence encoding for NS5 of the flavivirus sequence, whereby the at least one mutation results in inactivation of the 2′O-methyltransferase.

In a second aspect, there is provided a method of vaccination, comprising administration of at least one vaccine which is a mutated flavivirus comprising at least one mutation in a nucleic acid sequence encoding for NS5 of the flavivirus sequence, whereby at least one mutation results in inactivation of the 2′O-methyltransferase.

Other aspects and advantages of the invention will become apparent to those skilled in the art from a review of the ensuing description, which proceeds with reference to the following illustrative drawings of preferred embodiments.

### BRIEF DESCRIPTION OF THE DRAWINGS

The invention will be better understood with reference to the detailed description when considered in conjunction with the non-limiting examples and the accompanying drawings, in which:

FIG. 1 (a) depicts a computer generated surface representation of DENV-2 MTase structure, showing active site residues K61, K81, D146, and E217. SAH (S-adenosyl-L-homocysteine), a by-product of the methylation reaction, is shown in stick. The image was prepared using DENV-2 methyltransferase (MTase; PDB code: IL9K33) and PyMOL. (b) shows images of thin layer chromatography (TLC) plates and the effects of E217 and K61+E217A mutations on N7 and 2′-O MTase activities. Recombinant MTases were assayed for GpppA-RNA→m7GpppA-RNA and m7GpppA-RNA→m7GpppAm-RNA conversions to indicate N7 and 2′-O methylation activities, respectively. Relative methylation activities were indicated below the TLC images with wild type (WT) activity set as 100%. (c) is a series of micrographs of immunofluorescence analysis (IFA) in cells. BHK-21 cells were electroporated with equal amounts of WT and mutant genome length RNAs of DENV-2 and subsequently analyzed for viral protein E expression. At indicated days post-transfection, the cells were subjected to IFA using mouse antibody 4G2 against DENV E protein and anti-mouse IgG conjugated with FITC as primary and secondary antibodies, respectively. (d) shows photographs of the result of plaque assays. The plaque morphology of WT and mutant viruses recovered from viral RNA-transfected cells (passage 0), as well as the viruses after culturing in Vero cells for 10 rounds (passage 10) were analyzed by plaque assays. Both WT and mutant RNAs produced infectious viruses (passage 0) with similar plaque morphologies. Thus demonstrating that the infectivity of the mutant viruses is unaffected. (e) is a plot depicting the growth kinetics of viruses in different cell lines. Vero and mosquito C3/36 cells were infected with WT and mutant DENV-2 at an MOI of 0.1. Viral titers were measured at indicated time points using plaque assays. Average results of three experiments are presented.

FIG. 2 (a) shows an image of SDS-PAGE gel analyzing the DENV-1 and DENV-2 MTases that were expressed and purified. The recombinant proteins were analyzed on a 12% SDS-PAGE. DENV-1 and DENV-2 MTases contained the N-terminal 262 and 296 amino acids of NS5 protein, respectively. Molecular masses of protein markers are labeled. Amino acid E216 of DENV-1 MTase is equivalent to amino acid E217 of DENV-2 MTase. (b) shows a TLC plate, representing the effects of E216A and K61+E216A mutations of MTase on N7- and 2′-O methylation activities. Relative methylation activities were indicated below the TLC images with WT activity set as 100%. (c) shows pictures of immunofluorescence analysis (IFA) in BHK-21 cells. BHK-21 cells were transfected with equal amounts of WT and mutant genome-length RNAs of DENV-2. The cells were examined for viral E protein expression at indicated days post transfection. (d) shows images of cell-covered well plates to show plaque morphology of WT and mutant DENV-1 recovered from viral RNA-transfected cells (passage 0), as well as the viruses after culturing on Vero cells for 10 rounds (passage 10) were analyzed by plaque assays. (e) shows a series of graphs depicting the growth kinetics of DENV-1. Vero and C3/36 cells were infected with WT and mutant DENV-1 at an MOI of 0.1, and measured for viral yields at indicated time points. Average results of three experiments are presented.

FIG. 3 (a) shows detailed section of chromatograms obtained from DNA sequencing and data obtained from the indicated mutant virus passaged 10 times on Vero cells or (b) HEK-DC-SIGN cells. (c) Mice were infected with 2.75×10⁵ PFU of the indicated virus and viral RNA was isolated from plasma three days post-infection. Shown are sequences of RT-PCR products from the mutated region. The mutation sites are indicated with boxes. Thus FIG. 3 demonstrates the genetic stability of the E216/E217A mutation in vitro after repetitive passaging and in vivo after murine infection.

FIG. 4 (a) shows graphs depicting the viremia kinetics of AG129 mice infected with WT DENV-1 (strain West Pacific 74), DENV-1 K61A, and DENV-1 E216A or a combination of DENV-1 E216A and DENV-2 E217A in vivo. Mice were infected intraperitoneally (i.p.) with 2.75×10⁵ plaque forming units (pfu) of the indicated virus/mutant virus. Viral titers in the serum were measured at indicated time points by real-time PCR. (b) Viral titers in serum of mice vaccinated i.p. with 2.75×10⁵ pfu DENV-2 WT, DENV-2-E217A (strain TSV01) alone or in combination with or DENV1-E216A (2.75×10⁵ pfu DENV-1 E216A plus 2.75×10⁵ pfu DENV-2 E217A). Blood was taken at indicated time points and viral titers were measured by plaque assay. The dotted line represents the limit of detection. Each symbol represents one mouse. (c) shows graphs representing the viral titers in the plasma of mice vaccinated with 2′-O MTase mutant and challenged with the WT strains as indicated. Numbers in gray boxes indicate WT virus, whereas numbers in white boxes indicate 2′-O MTase mutant virus. Mice were vaccinated i.p. with 2.75×10⁵ pfu of the indicated 2′-O MTase mutant serotype and challenged 30 days later with 5×10⁵ pfu WT DENV-1 strain (strain 05K3126 used for challenge due to its high virulence in mice) or 3×10⁶ pfu WT DENV-2. Blood was taken at indicated time points and viral titers were measured by plaque assay. ND: not detected. (d and e) are scatter plots depicting the IgG titers of mice vaccinated and challenged, as described above. Blood was taken at indicated time points post-challenge and IgG antibody titers against DENV-1 (d) and DENV-2 (e) were measured by ELISA. Data are representative of two experiments with three to four mice per group in each experiment (a, b) or two pooled experiments (c-e) with a total of 9 mice per group. Bars represent means with SD (a) or means with SEM (b-e). Thus, FIG. 4 demonstrates that dengue MTase mutants are attenuated and immunogenic.

FIG. 5 (a) is a contour plot obtained by flow cytometry of intracellular IFN-γ measured in spleen CD4 and CD8 cells (lymphocyte gate, viable cells, cell doublets excluded) of unvaccinated or vaccinated mice; representative graphs for each group are shown. (b) Shows box plot graphs showing quantitative analysis of IFN-γ production. Bars are means±SEM from two independent experiments with 2-3 mice per group in each experiment. P value was determined with an unpaired student's t test. Splenocytes of IFNAR mice infected with DENV-2 E217A or DENV-2 WT were harvested at day 7 and were re-stimulated with DENV-2 virus or with NS4B and NS5 peptides for the quantification of IFN-γ production in CD4 and CD8 cells, respectively. Thus, FIG. 5 demonstrates that T cell IFN-γ production is elicited by 2′-O-MTase mutant DENV-2.

FIG. 6 (a) shows a survival chart of mice that were vaccinated intraperitoneally (i.p) with 2.75×10⁵ pfu DENV-1 WT, DENV-1 E216A, DENV-2 WT, DENV-2 E217A (strain TSV01) alone or in combination with DENV-1 E216A (2.75×10⁵ pfu DENV-1 E216A plus 2.75×10⁵ pfu DENV-2 E217A), or were unvaccinated (PBS). Thirty days post-vaccination, mice were challenged intraperitoneally with 10⁷ pfu of the virulent DENV-2 strain, D2Y98P, and the health status monitored twice daily. (b) shows a graph representing the viral titers measured by real-time PCR in blood taken at indicated time points. (c) shows a column graph of TNF-α levels in plasma of mice, which was measured at day three post-challenge according to the manufacturer's protocol (eBioscience). Data represent means±SEM from 3 experiments with a total of 7-10 mice (a) or means±SEM from two experiments with a total of 6-8 mice (b-c). Statistical analysis was performed using 1-way ANOVA Tukey's multiple comparison test (***P<0.001). Thus, FIG. 6 demonstrates that 2′-O MTase mutant protects against challenge with an aggressive mouse-adapted DENV-2 strain.

FIG. 7 (a) shows a graph depicting the percentage of infected cells in culture. Cells were seeded in a 24-well plate, treated for 24 h with increasing amounts of IFN-β and infected with DENV-2 WT or E217A DENV-2. At 72 h post-infection, cells were harvested and analyzed by flow cytometry using 4G2 antibody (against viral envelope protein). (b) shows a graph representing viral titers in culture fluids measured by plaque assay. Data are representative of three experiments. Means and SD are shown. Statistical analysis was performed using Student's t-test (***, p<0.001; *, p<0.05). (c) HEK293-DC-SIGN cells were transiently transfected with vector alone, human IFIT-1 (ISG56), IFIT-2 (ISG54), IFIT-3 (ISG60), or IFIT-5 (ISG58). On day 2 post-transfection, cells were infected with DENV-2 WT or E217A DENV-2 at an MOI of 5. The cells were analyzed for viral envelope protein expression by flow cytometry at 72 h post-infection. Results represent the mean±SEM of six independent experiments. Percentage of infected cells was normalized to cells transfected with empty vector. (d) shows column graphs showing virus output from transfected cells determined in the supernatant by plaque assay. The transfection efficiency was 30-50%, (determined by parallel experiments with a Green Fluorescent Protein (GFP) expression plasmid). (e) shows a line graph depicting the growth kinetics of E217A DENV-2 and DENV-2 WT in HEK293-DC-SIGN cells. Statistical analysis was performed using one-way ANOVA Bonferroni's multiple comparison test (**, p<0.01). Accordingly, FIG. 7 demonstrates that 2′-O MTase mutant DENV-2 has altered sensitivity to IFN-β, which is partially mediated by IFIT1.

FIGS. 8 (a), (b) and (c) show graphs showing results of plasma analysis from AG129 mice analyzed 30 days after vaccination with mutant or wild-type DENV virus. Upper graphs in panels (a), (b) and (c) show antibody-dependent enhancement (ADE) assays using K562 cells and lower graphs show the corresponding neutralization assay using U937-DC-SIGN as target cells. Groups of mice were vaccinated with (a) DENV-1 E216A, DENV-1 WT, DENV-1 E216A and DENV-2 E217A combined or PBS; (b) DENV-2 E217A or DENV-2 WT. (c) shows graph depicting the rates of infection as well as normalized infection based on the level of antibody 4G2, which was used as a technical control. Symbols in panels (a) and (b) are the means±SEM of three mice per group, tested in duplicate. The shown experiment is representative for one of two. The mean±SD from the two independent experiments (n=3-4 per group) are shown in Table 1.

FIG. 9 (a) is a set of graphs depicting DENV-1 or DENV-2 in the presence of serum of infected K562 cells, diluted as indicated in the x axes. Symbols are means±SEM of three sera per group from two independent ADE assays testing the sera in duplicate each. (b) is a graph that shows the same sera as in (a) tested for neutralization by using U937-DC-SIGN as target cells. Symbols are means±SD of three sera per group, tested in duplicate each. (c) and (d) are a set graphs showing the detection of infected cells using 4G2 antibody as a technical control for the infection of (c) K562 cells or (d) U937-DC-SIGN cells. Symbols are means±SD of duplicate values. The serum of three monkeys per group was analyzed for ADE activity. Sera from day 5 after challenge with DENV-2 WT virus in unvaccinated animals (day 5 post-infection) or 5 days after challenge in animals vaccinated with E217A DENV-2 virus 64 days earlier (day 5 post-challenge).

FIG. 10 (a) shows column graphs representing HEK293-DC-SIGN cells and (b) U937-DC-SIGN cells, which were seeded in a 24-well plate, incubated for 24 hours with 0, 20 or 200 IU/ml of IFN-β and infected at an MOI of 1 with E217A or WT DENV-2. 48 hours post-infection the percentage of infected cells was determined by flow cytometry. 100 μl of the supernatant (passage p1) was transferred to newly seeded IFN-β pre-treated cells. The remaining supernatant was kept for isolation of viral RNA and sequencing. This procedure was repeated two more times (p2 and p3). P3 was collected after 96 instead of 48 hours to allow any potential mutants to have enough time to grow to high titers. Thus, FIG. 10 demonstrates that E217A does not mutate and escape IFN-β pressure in human cell lines HEK293-DCSIGN and U937-DC-SIGN.

FIG. 11 is a scatter plot that shows data of ten female mosquitoes inoculated intrathoracically with 0.17 μl of DENV-2 WT or E217A DENV-2 at a titer of 10⁵ pfu/ml. Seven days later mosquitoes were killed by freezing and homogenized. Viral RNA was quantified by real-time qRT-PCR. Mean and 95% CI intervals are indicated by horizontal bars, each point represents a single female mosquito. P=0.105, unpaired t test. FIG. 11 demonstrates that the genome copy number of the WT virus was approximately 35% higher than that of the mutant virus (p=0.1054). Overall, the results demonstrate that the 2′-O-MTase mutant virus is compromised in vector fitness.

FIG. 12 is a set of graphs showing plotted growth curves for WT and double mutant strains of DENV-1, DENV-2, DENV-3 and DENV-4 in C6/36 cells up to six days post-infection. Cells were infected with an MOI of 0.01 and the virus quantified using plaque assay. Data are means±SD of three independent experiments.

FIG. 13 is a set of graphs showing plotted growth curves for WT and double mutant strains of DENV-1, DENV-2, DENV-3 and DENV-4 in Vero cells up to six days post infection. Cells were infected with a MOI of 0.01 and the virus quantified using plaque assay. Data are means±SD of three independent experiments.

FIG. 14 is representative pictures of cells of 24-well plates showing plaque morphology of stained Vero cells infected with double mutant DENV-3 or DENV-4 virus. The double mutant viruses recovered from viral RNA-transfected cells (passage 0) as well as the virus after culturing on Vero cells for 5 rounds (passage 5) were analyzed by plaque assay.

FIG. 15 is a bar graph depicting infected cells analyzed by flow cytometry. U937-DC-SIGN cells were pre-treated with increasing concentrations of 0, 2, 20 and 200 U of IFN-β, 24 h before infection with double mutant DENV strains (white bars) or wild type DENV virus (black bars). The percentage of infected cells under each condition was analyzed by flow cytometry 24 h after infection.

FIG. 16 is a set of graphs depicting the growth kinetics of wildtype and mutant viruses in AG129 mice. Mice were infected with 10⁵ pfu wildtype of double mutant DENV-1, DENV-2 or DENV-4, or with 3.3×10⁴ pfu wildtype or double mutant DENV-3 and blood was collected at day 1, 3, 5 and 7 after infection for detection of viral RNA with qRT-PCR.

FIG. 17 is a set of graphs showing the immunogenicity of wildtype versus mutant viruses by measuring (a) end-point titers of DENV-specific antibodies and (b, c) neutralizing titers in mice vaccinated with double mutant DENV1, 2, 3 and 4 viruses or the respective WT viruses. ELISA plates were coated with UV-inactivated whole virus particles of DENV1, 2, 3 or 4 and plasma was added at decreasing concentrations to determine the end-point titer of DENV-specific antibodies. Each symbol represents one mouse. Means±SD are shown. B-C) Neutralizing titers of three mice per group were measured in a flow-cytometry based assay. B) NT50 values for plasma from mice infected with the indicated WT of MT viruses. Each symbol represents one mouse.) One mouse in the DENV-3 MT group and two mice in the DENV-4 group had neutralizing titers that were too low for an accurate curve fit and the NT50 values were arbitrarily set to 10 for illustration purpose. C) Average neutralization curves per mouse group. Mouse sera were diluted 1:5.0 to 1:12′150 and incubated with DENV1, 2, 3 or 4 according to the infection serotype before infection of U937-DC-SIGN cells as described in Materials and Methods. The curves are means±SEM for three mice per group, each plasma sample measured in duplicates.

FIG. 18 is a set of bar graphs showing virus titers in mice vaccinated with double MT mutant DENV, wildtype DENV or unvaccinated mice (PBS) and challenged with wildtype DENV. Each dot represents one mouse and bars show means±SD. Thirty days after vaccination with double mutant DENV-MT, DENV-WT or PBS, the mice were challenged with wildtype DENV virus, using different strains than the ones used for vaccination. Challenge dosages were as follows: WT DENV-1: 2×10⁷ pfu/mouse, WT DENV-2: 1×10⁷ pfu/mouse, WT DENV-3: 2×10⁷ pfu/mouse, WT DENV-4: 1.6×10⁸ pfu/mouse. At day 3 after challenge, the virus titer in the blood of the mice was assessed by qRT-PCR to test whether the mice were protected.

### DETAILED DESCRIPTION OF THE PRESENT INVENTION

Dengue is prevalent in densely populated areas in tropical countries. Progressive urbanization in Asia and South America has accelerated the global expansion of dengue-endemic areas and this has resulted in a continuous increase in the number of cases, despite this no vaccine is available yet. Due to the limitations of current vaccine candidates in clinical testing, development of “second generation” vaccines is needed. Viruses defective in 2′-O methylation are attenuated in vitro and in vivo.

Accordingly, the inventors developed flavivirus virus mutants, such as dengue virus mutants lacking 2′-O methyltransferase (2′-O MTase) disclosed herein. As explained in more detail in some of the examples below, the flavivirus mutants are highly sensitive to type I interferon, are attenuated in mice and rhesus monkeys and elicit a strong adaptive immune response. Targeting conserved amino acid sequences between various serotypes of a given flavivirus contributes to the development of a vaccine inducing protection against all types of Dengue borne diseases.

Live attenuated vaccines are replication-competent viruses that can induce an immune response without causing disease. Prominent examples of successful live attenuated vaccines that provide long-term immunity are vaccinia virus, poliovirus (Sabin), and two members of the Flaviviridae, namely yellow fever virus (YF-17D) and Japanese encephalitis virus (JEV). Live-attenuated DENV vaccines have been shown to induce protective neutralizing antibody titers in mice, monkeys and humans. In addition, evidence that a balanced T cell response contributes to protection is accumulating. In, a human challenge model, where participants were vaccinated with a tetravalent, live attenuated vaccine strain followed by challenge with DENV-1 or -3, those individuals who were protected showed a sustained IFN-γ response. Live attenuated vaccines include natural DENV T cell epitopes and efficiently trigger both CD4 and CD8 T cells via infection of antigen-presenting cells.

Flaviviruses replicate in the cytoplasm. The cytoplasm-replicating viruses have evolved N7- and 2′-O-methyltransferases (MTase) to methylate their viral mRNA 5′ cap structures. Surprisingly, the inventors found that while 2′-O MTase is not essential for viral replication in vitro, viruses bearing mutations in the highly conserved methyltransferase catalytic K-D-K-E tetrad are severely attenuated in the host, due to the inability of the virus to shield viral RNA from recognition by host innate immune factors.

Thus, in a first aspect, there is provided a method of eliciting an immune response comprising administration of a mutated flavivirus comprising at least one mutation in a nucleic acid sequence encoding for NS5 of the flavivirus sequence, whereby the at least one mutation results in inactivation of the 2′O-methyltransferase.

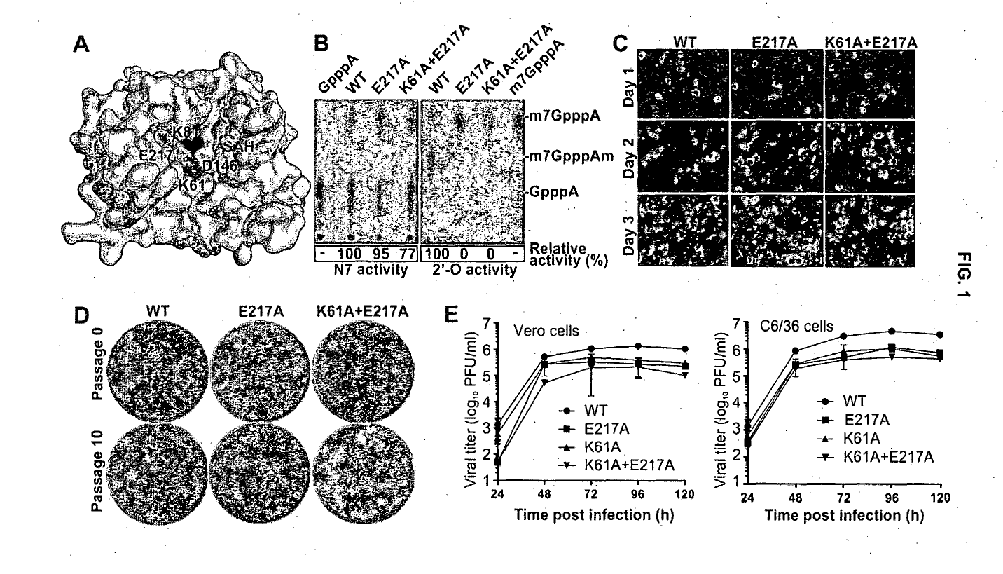

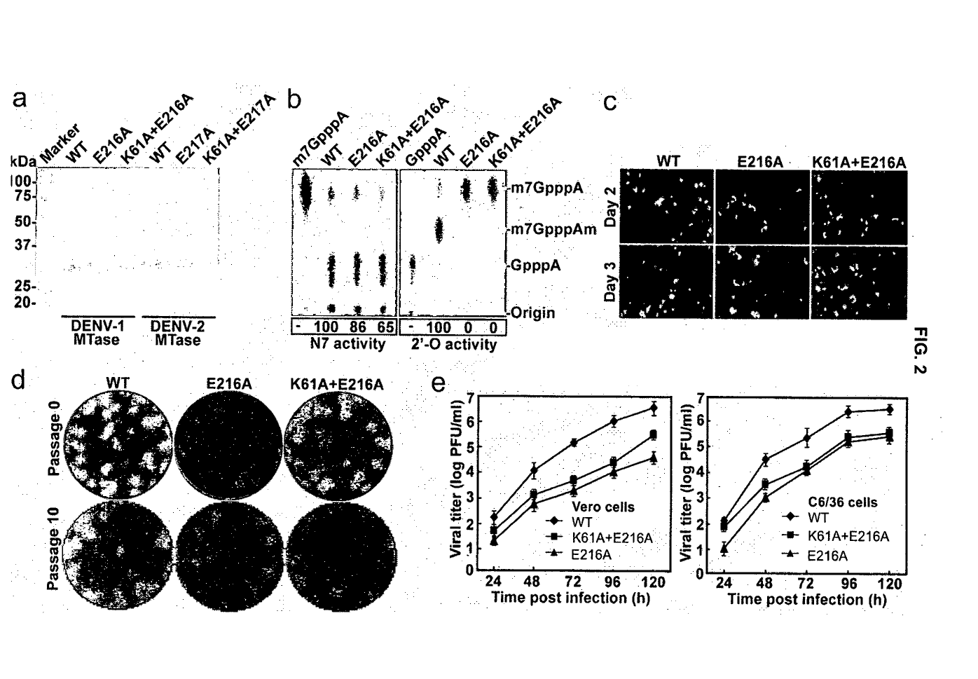

The inventors have shown, as exemplified in the examples below, such as example 1 and 2 and FIG. 1, that the amino acid of the highly conserved catalytic motif KDKE tetrad 2′-O MTase are essential for methylation of their own viral genomic nucleic acid. Accordingly, the viral nucleic acid of the flavivirus is shielded from recognition by the host innate immune factors that interact with downstream signaling molecules and activate an antiviral cascade.

As used herein, the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double-stranded (duplex). Duplex nucleic acids may be homoduplex or heteroduplex.

As used herein, the term “mutation” or grammatical variants thereof, in general relates to an altered genetic sequence which results in the gene coding for a non-functioning protein or a protein with substantially reduced or altered function. In the present context, the term “mutation” also relates to a modification of the genome or part of a nucleic acid sequence of any biological organism, virus or extrachromosomal genetic element. The mutation can be performed by replacing one nucleotide by another in the viral nucleic acid sequence, thus creating a different amino acid. The technique used may comprise alanine scanning mutagenesis for example. Such techniques are well known to the person skilled in the art. It allows by using PCR, a set of primers and a vector comprising a sequence of interest to create changes in nucleotide sequences at desired positions. The mutation can be induced artificially using, but not limited to, chemicals and radiation, but can also occur spontaneously during nucleic acid replication in cell division. Some mutations may result in a premature stop codon. When artificially created, in the context of the invention, a mutation is by extension, the replacement of an amino acid encoded by a given nucleic acid sequence to another amino acid in a flavivirus. Thus, the virus carrying a mutation is referred to as a mutant virus in reference to a wild-type virus. The wild-type virus thus refers to a virus that serves as a reference for example, in light of the exemplary genomic sequences found in databases known to the person skilled in the art.

For example, the nucleotide sequences may be mutated such that the activity of the encoded proteins in vivo is abrogated. In another example the nucleotide sequences may be codon optimized, for example the codons may be optimized for human use. In preferred examples, the nucleotide sequences of the invention are both mutated to abrogate the normal in vivo function of the encoded proteins, and codon optimized for human use.

As regards codon optimization, the nucleic acid molecules of the invention have a nucleotide sequence that encodes the proteins of the invention and may be designed to employ codons that are used in the genes of the subject in which the antigen is to be produced. Many viruses, including flaviviruses, use a large number of rare codons and, by altering these codons to correspond to codons commonly used in the desired subject, enhanced expression of the proteins, may be achieved. In one example, the codons used are “humanized” codons, i.e., the codons are those that appear frequently in highly expressed human genes, instead of those codons that are frequently used by flaviviruses. Such codon usage provides for efficient expression of the recombinant flaviviruses proteins in human cells. Any suitable method of codon optimization may be used. Such methods, and the selection of such methods, are well known to those of skill in the art. Thus, the nucleotide sequences of the invention may readily be codon optimized.

The invention further encompasses nucleotide sequences encoding functionally and/or antigenically equivalent variants and derivatives of the viruses and antigens of the invention and functionally equivalent fragments thereof. These functionally equivalent variants, derivatives, and fragments display the ability to retain the capacity to elicit an immune response against the virus and antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs, are those which will not significantly affect properties of the encoded virus or polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one example, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the virus, antigen, epitope, immunogen, peptide or polypeptide of interest. For example, it is well known to the person skilled in the art that flaviviruses, such as dengue viruses, may have numerous sequences that mutate according to geographic locations and time. Thus, the method as described herein may also be useful to elicit an immune response comprising administration of mutated flaviviruses, whose nucleotide sequence vary from the exemplified sequences described herein. For the purposes of the present invention, sequence identity or homology is determined by comparing the sequences when aligned so as to maximize overlap and identity while minimizing sequence gaps. In particular, sequence identity may be determined using any of a number of mathematical algorithms.

The term “recombinant” when referring to a molecular species, such as a nucleic acid or protein, indicates that the material (e.g., a nucleic acid or protein) has been synthetically (non-naturally) altered by human intervention. The alteration to yield the synthetic material can be performed on the material within or removed from its natural environment or state. For example, a naturally occurring nucleic acid is considered a recombinant nucleic acid if it is altered, or if it is transcribed from DNA which has been altered, by means of human intervention, e.g., performed on the cell from which it originates. By extension, a mutated flavivirus is a flavivirus, whose genome has been mutated.

Sequence analysis can also be used to detect specific mutations in flaviviruses. Therefore, in one example, determination of the presence or absence of a mutation in a flavivirus of interest entails directly sequencing DNA or RNA obtained from a subject. If desired, PCR is used to amplify a portion of a nucleic acid encoding the flavivirus genome, and the presence of a specific mutation is detected directly by sequencing the relevant site(s) of the DNA or RNA in the sample.

Mutations in the NS5 coding sequence such as in the 2′-O MTase coding sequence may lead to altered expression levels, e.g., a decrease in the expression level of an mRNA or protein, which leads to an abnormal phenotype. Such mutations are detected via, e.g., ELISA, radioimmunoassays, immunofluorescence, Northern blotting, and Western blotting to compare 2′-O MTase expression levels in a subject to a biologically-matched control or reference. These detection processes are described in the art.

Any method of detecting mutant proteins is appropriate for use in the context of the invention, and many are known in the art. For example, 2′-O MTase may be isolated from a cellular, sample and subjected to amino acid sequencing, the results of which are compared to a reference amino acid sequence. Mutant 2′-O MTase also can be identified by detecting altered molecular weights compared to wild-type 2′-O MTase using gel electrophoresis (e.g., SDS-PAGE). Immunoassays, e.g., immunofluorescent immunoassays, immunoprecipitations, radioimmunoasays, ELISA, and Western blotting, also can be used. Examples of specific point mutations in the NS5 2′-O-MT are given below.

It should be understood that the proteins, including the 2′-O MTase may differ from the exact sequences illustrated and described herein. Thus, the invention contemplates deletions; additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described, but possessing minor amino acid substitutions that do not substantially affect the immunogenicity of the protein are, therefore, within the scope of the invention.

Thus, the method described herein, may comprise a mutated flavivirus, wherein there are at least two mutations, which lead to the inactivation of the 2′O-methyltransferase. In one example, there is provided the mutated flavivirus as described herein, wherein the at least one amino acid is a polar amino acid. The polar amino acid may be involved in the catalytic activity of a protein of the flavivirus of the invention that contributes to the virulence of said virus. Thus, replacing the polar amino acid with another amino acid, for example, a non-polar amino acid may help reduce, abrogate, prevent or inhibit the activity of the enzyme. The catalytic motif KDKE of NS5 of the flavivirus contains such polar amino acids. Thus, in one example, there is provided the method as described herein, wherein the at least one mutation or the at least two mutations are in the KDKE motif. In a further example, there is provided the method as described herein, whereby the mutations result in replacement of a polar amino acid in the KDKE motif of NS5 of the flavivirus. In one example, the mutated flavivirus comprises one or two or three or four point mutations in the KDKE motif.

Since the invention provides for a method of eliciting immune response, it is understood that any mutations elsewhere in the flavivirus genome that abrogates the pathological conditions in the host after administration of the mutated flavivirus may be of interest. For example, there is provide the method as described herein, wherein the mutated flavivirus comprises at least one, at least two, at least three, at least four or more further mutations in a motif comprising, but not limited to, a GTP-pocket, a SAM-pocket and a RNA binding site of the non-structural protein 5 of the flavivirus.

Accordingly, in a further example, there is provided the method as described herein, wherein the further mutation results in replacement of a polar amino acid in the GTP-pocket, and/or SAM-pocket and/or RNA binding site of the non-structural protein 5 of the flavivirus. As described above, any suitable mutation can be envisaged as long as the flavivirus maintains its immunogenic capacity but loses its pathogenic potential. In other words, a mutation may affect the function of a protein that contributes to the ability of the flavivirus to incur a disease or pathology in an infected host. The proteins of the flavivirus that can be mutated may be involved, for example, in replication, in methylation, in RNA metabolism, in transport of the virus, in metabolism, in infection or in any other function that allows the flavivirus to contribute to the pathology associated with the infection of the host.

The at least one, at least two, at least three, at least four, at least five or more mutations may or may not contribute to the inactivation of the 2′O-MTase of the NS5 of the flavivirus. The mutations may be point mutations, i.e. one nucleic acid mutation corresponds to the change of one amino acid. For example, the mutations may comprise, but are not limited, to one mutation, two mutations, three mutations, four mutations, five mutations or more, mutations resulting in the replacement of one, two, three, four, five or more amino acids. The mutation may be a deletion, an insertion, a point mutation or a combination thereof. Example of specific point mutations is given in the examples herein below.

In one example, there is provided the mutated flavivirus as described herein, wherein the at least one mutation results in the replacement of a polar amino acid with a non-polar amino acid at Lysine 61 (K61), or Lysine 81 (K81), or glutamic acid 217 (E217) or equivalent respective amino acid positions in the KDKE motif of NS5 of the flavivirus. Thus, in one example, there is provided the method as described herein, wherein the at least one mutation results in the replacement of a polar amino acid with a non-polar amino acid at Lysine 61, or Lysine 181, or glutamic acid 217 or equivalent respective amino acid positions in the KDKE motif of NS5 of the flavivirus. As will be described in more detail below, the above-mentioned amino acids are essential amino acid for the function of the 2′-O methyltransferase. In the specific example above, the dengue virus DENV-2 (having the polyprotein amino acid sequence of SEQ ID NO: 2) or DENV-4 (having the polyprotein amino acid sequence of SEQ ID NO: 4) will have their 2′O-MT activity abrogated by such mutations. The mutation may be at Lysine 61, or Lysine 81, or Glutamic acid 217 or a combination thereof. An equivalent respective amino respective position for E217 in the NS5 protein of the DENV-1 (having the polyprotein amino acid sequence of SEQ ID NO: 1) or DENV-3 (having the polyprotein amino acid sequence of SEQ ID NO: 3) dengue virus is E216 (glutamic acid 216 at position 216 starting from the first amino acid of the NS5 protein of DENV-1).

In one example, there is provided the method as described herein, wherein the mutations that result in the replacement of a polar amino acid with a non-polar amino acid is the amino acid at Lysine 61 and Glutamic acid 217, or at equivalent respective positions in the KDKE motif of NS5 of the flavivirus. In yet another example, there is provided the method as described herein, wherein the further mutation in the GTP-pocket is at Lysine 14 and/or Lysine 29 or at equivalent respective amino acid positions in the GTP-pocket of NS5 of the flavivirus. The mutations, as described above in the GTP-pocket of NS5 of the flavivirus, may affect the 2′-O methylation ability of the protein.

Another useful mutation may be in the SAM-binding pocket. For example, mutation of the isoleucine at position 147 of NS5 of the flavivirus may also affect the 2′-O methylation activity of the protein. Therefore, in one example, there is provided the method as described herein, wherein the further mutation in the SAM-pocket is at Isoleucine 147 or at equivalent respective amino acid positions in the SAM-pocket of NS5 of the flavivirus.

The RNA-binding site of NS5 of the flavivirus may be mutated for example at position Glutamic acid 35 and/or Tryptophan 87. Mutation of Glutamic acid 35 and/or Tryptophan 87 in a flavivirus such as dengue virus also affects the 2′ O-methylation activity of NS5. In one example, there is provided the method as described herein, wherein the further mutation in the RNA binding site is at Glutamic acid 35 and/or Tryptophan 87 or at equivalent respective amino acid positions in the RNA-binding site of NS of the flavivirus.

The mutations in NS5 of the flavivirus may be combined to further inactivate the activity of the protein. Thus, the disclosure provides for mutated flaviviruses having at least two mutations or two mutations, as described above and herein. In the example below, it will be evident that some combinations of mutations improve the inactivation of the enzymes. For example, as described in more details in the examples below, if both the K61 and E217 are replaced by alanine in the 2′O-MT of a DENV-2 dengue virus, the activity of the enzyme is greatly diminished, when compared to the non-mutated enzyme. Other combinations are described in the examples and the Table below. The replacement of one amino acid with another is known to the skilled artisan, and may include manipulating the nucleic acid to mutate the sequence of the gene of interest to modify the amino acid that may be encoded.

In one example, there is provided the method as described herein, wherein when there are at least two mutations, at least two amino acids are replaced with non-polar amino acid at positions comprising, but not limited to Lysine 61, Lysine 181, Glutamic acid 216, and equivalent respective amino acids positions in the KDKE motif.

Thus, there is provided the method as described herein, wherein further mutations comprise mutations at positions comprising, but not limited to Lysine 14 and Lysine 29 in the GTP-pocket, Isoleucine 147 in the SAM-pocket, Glutamic acid 35 and Tryptophan 87 in the RNA binding site and equivalent respective amino acids positions.

In a further example, there is provided the method of any of the preceding claims, wherein the mutated flavivirus has three mutations in the nucleic acid sequence encoding for a non-structural protein 5 of the flavivirus sequence, whereby the three mutations result in inactivation of the 2 ‘O-methyltransferase.

As explained above in some of the examples below, the inventors characterized the N7- and 2’-O methylation activity by mutating the amino acids of the KDKE tetrad and surprisingly found that such a mutation abolished the 2′-O methylation activity of the 2′-O MTase of NS5 of the flavivirus. The N7-methylation activity was reduced. Advantageously, the activity of the same MTase was abolished in all the four serotypes of DENV when a mutation of the amino acids of the KDKE tetrad was performed to replace at least one polar amino acid with a non-polar amino acid.

The term “equivalent respective amino acid position” as used herein refers to identical or conserved amino acid between different viruses or serotypes of a given flavivirus having the same functional or structural position. For example, the glutamic acid at position 216 in the NS5 protein of serotype DENV-1 of dengue virus is an equivalent respective amino acid position of the glutamic acid at position 217 in the NS5 protein of the serotype DENV-2 of dengue virus in the KDKE motif. The position is in reference to the first amino acid (N-terminal) of the 2′-O methyltransferase of the NS5 protein of SEQ ID NO: 1 and SEQ ID NO: 2, respectively.

In another example, there is provided the mutated flavivirus as described herein, wherein the at least one mutation that results in the replacement of a polar amino acid is the amino acid at Lysine 61 of the non-structural protein 5 of the flavivirus. In another example, there is provided the mutated flavivirus as described herein, wherein the at least one mutation that results in the replacement of a polar amino acid is the amino acid at Lysine 61 or Glutamic acid 217 in the KDKE motif of NS5 of the flavivirus. As shown in some of the examples below, the replacement of either K61 or E217 or a combination of K61 and E217 to alanine is efficient in abrogating/inhibiting or at least diminishing, the 2′-O methylation activity of the enzyme (e.g. example 1).

In some examples, there is provided the mutated flavivirus as described herein, wherein NS5 of the flavivirus may have two mutations resulting in the expression of an amino acid whereby two amino acids are replaced with a non-polar amino acid at two positions comprising, but not limited to, Lysine 61 or Lysine 81 or glutamic acid 216 or glutamic acid 217 or equivalent respective amino acids in the KDKE motif.

In further examples, there is provided the flavivirus as disclosed herein, wherein in case there is only one mutation, at least one or at least two or at least three or more further mutations can be comprised that results in the expression of an amino acid at a position comprising, but not limited to, Lysine 61, Lysine 81, glutamic acid 217, Lysine 14, Lysine 29, Isoleucine 147, Glutamic acid 35, Tryptophan 87 and equivalent respective amino acids in the KDKE motif, GTP-pocket, SAM-pocket or RNA binding site.

The GTP-pocket, SAM-pocket and RNA binding sites have been identified as being potential crucial sites for the enzymatic activity of NS5 of the flavivirus. As mentioned above, NS5 is highly conserved among members of the flavivirus, thus important structural and functional amino acids of the S-adenosyl-L-methionine dependent methyltransferase (SAM) domain, and the key amino acids of the domains possessing the methyltransferase and guanylyl transferase activity may be mutated as well. Additionally, the amino acid responsible for the RNA binding to the enzyme may be mutated to alter the modification of the RNA.

Thus, in another example, there is provided the mutated flavivirus as described herein, wherein NS5 of the flavivirus has two mutations resulting in the expression of an amino acid whereby two amino acids are replaced with a non-polar amino acid at two positions comprising, but not limited to, Lysine 61, Lysine 81, glutamic acid 216, glutamic acid 217, and equivalent respective amino acids in the KDKE motif. In one example, there is provided the flavivirus as described herein, wherein the group further comprises Lysine 14, Lysine 29, Isoleucine 147, Glutamic acid 35, Tryptophan 87 and equivalent respective amino acids in the KDKE motif, GTP-pocket, SAM-pocket or RNA binding site.

In a further example, there is provided the flavivirus as described herein, wherein the two amino acids are the amino acids at Lysine 61 or Glutamic acid 216 in the KDKE motif of NS5 of the flavivirus. In one example, there is provided the flavivirus as described herein, wherein the two amino acids are the amino acids at Lysine 61 or Glutamic acid 217 in the KDKE motif of NS5 of the flavivirus. Advantageously, the mutations of the invention result in the inactivation or reduction or abolition or inhibition of the catalytic activity of the enzyme as disclosed herein, such as 2′-O MTase.

In one example, there is provided the method as described herein, wherein when there are at least two mutations, at least two amino acids are replaced with non-polar amino acid at positions comprising, but not limited to Lysine 61, Lysine 181, Glutamic acid 216, and equivalent respective amino acids positions in the KDKE motif Possible double mutations may comprise a flavivirus, such as the dengue virus having K61A/K181A mutations, K61A/E216A mutations, K181A/E216A mutations, K61A/E217A mutations, or K181A/E217A mutations. The mutations may result in an absent or inhibited 2′-OMTase activity of the NS5 protein of the flavivirus.

In one example, there is provided a mutated flavivirus comprising a nucleic acid sequence wherein at NS5 of the flavivirus sequence at least one mutation results in an expression of an amino acid whereby at least one amino acid is replaced with a non-polar amino acid in the GTP-pocket, SAM-pocket or RNA binding site of NS5 of the flavivirus. In other examples, there is provided the mutated flavivirus as described herein, wherein the at least one amino acid comprises, but is not limited to, amino acids at Lysine 14, Lysine 29, Isoleucine 147, Glutamic acid 35, Tryptophan 87 or equivalent respective amino acids in the GTP-pocket, SAM-pocket or RNA binding site of NS5 of the flavivirus. Thus, in another example, there is provided a method as described herein, wherein further mutations comprise mutations at positions comprising, but not limited to Lysine 14 and Lysine 29 in the GTP-pocket, Isoleucine 147 in the SAM-pocket, Glutamic acid 35 and Tryptophan 87 in the RNA binding site and equivalent respective amino acids positions.

In another example, there is provided the method as described herein, wherein the mutated flavivirus has three mutations in the nucleic acid sequence encoding for NS5 of the flavivirus sequence, whereby the three mutations result in inactivation of the 2′O-methyltransferase.

It is to be understood that all the exemplary mutations of the nucleic acid sequence of the NS5 of the flavivirus described herein, may result in a virus that is still capable of eliciting an immune response in the host. In other words, the mutated virus may be an attenuated virus and may be used as an immunogen. Thus, in one example, there is provided the mutated flavivirus as described herein, wherein the flavivirus is an attenuated virus. Accordingly, there is provided the method as described herein, wherein the flavivirus is an attenuated virus.

The inventors demonstrated in some of the examples below that exemplary viruses as described herein, such as mutated dengue viruses, are attenuated viruses. The viruses have lost their pathological abilities, i.e. they do not induce the diseases typically associated with virulent dengue viruses when administered in a host.

Moreover, advantageously, no spontaneous mutations were observed in the mutated viruses when cultured for a number of passages on Vero Cells. That is, after for example 5 passages, no spontaneous mutations were observed in the virus 2′O MTase that would revert to the WT form or reactivate the enzyme. Thus, there is evidence of the genetic stability of mutated flaviviruses such as dengue viruses after passaging in vitro.

The present disclosure provides evidence in the examples below that the mutated flavivirus, such as the dengue virus of the invention is highly attenuated in mice and non-human primates. For example, a mutated dengue virus as described herein induces a broad and protective immune response. The inventors demonstrated that the dengue virus as disclosed herein is safe, as injection does not cause a flavivirus-related disease, is effective in its ability to induce a neutralizing antibody response, which protects against challenge with virulent WT virus.

As used herein, the term “attenuated virus” is a viable (“live”) virus, in which the virulence of the infectious agent has been reduced, e.g. though passaging the virus in a specific cell line, or through genetic manipulation of the viral genome. The attenuation of the virus pertains to its virulence (pathogenicity), but does not necessarily affect the replicative capability of a virus. An attenuated virus can still be capable of replication. Thus, it may be a strain of a virus whose pathogenicity has been reduced so that it will initiate the immune response without causing the specific disease. In the context of the present invention, an attenuated virus may be a flavivirus whose pathogenicity has been abrogated or reduced by inactivating at least one viral enzyme involved in virulence. Examples of such enzymes may include an enzyme that allows the virus to escape from the host immune detection such as 2′-O MTase, as described in more details in the examples below or an enzyme involved in the replication of the virus. An attenuated virus is a viable virus in which the virulence of the infectious agent has been reduced, e.g. though passaging the virus in a specific cell line, or through genetic manipulation of the viral genome.

The mutated flavivirus as described herein may be an inactivated virus. The term “inactivated” in the context of a dengue virus vaccine means that the virus is incapable of replication in vivo or in vitro. For example, the term inactivated may refer to an attenuated virus that has been replicated, e.g., in vitro, and then deactivated using chemical or physical means so that it is no longer capable of replicating. The term can also include antigens produced by further processing (e.g., splitting, fractionation, and the like), and components produced by recombinant means, e.g., in cell culture.

As used herein, the terms “antigen” or “immunogen” are used interchangeably to refer to a compound, composition, or substance that can stimulate the production of antibodies and/or a T cell response in an animal, including compositions that are injected, absorbed or otherwise introduced into an animal. The term “antigen” includes all related antigenic epitope substances, typically a protein, which is capable of inducing an immune response in a subject. The term also refers to proteins that are immunologically active in the sense that once administered to a subject (either directly or by administering to the subject a nucleotide sequence or vector that encodes the protein) it is able to evoke an immune response of the humoral and/or cellular type directed against that protein.

In some examples, the flavivirus as described herein is a dengue virus of any serotype or a tick borne encephalitis virus (TBEV) of any serotype. In some examples, the mutated flavivirus as described herein is a dengue virus. Thus, in one example, there is provided the method as described herein, wherein the flavivirus is a dengue virus.

In a further example, the mutated flavivirus, as described herein, is a dengue virus comprising at least one or at least two or at least three or at least four or more dengue virus ribonucleic acid sequences that may comprise, but is not limited to, a dengue virus 1 ribonucleic acid sequence (DENV-1), a dengue virus 2 ribonucleic acid sequence (DENV-2), a dengue virus 3 ribonucleic acid sequence (DENV-3) and a dengue virus 4 ribonucleic acid sequence (DENV-4). The cDNA can be obtained from the flavivirus ribonucleic acid sequence and the cDNA can be cloned in an appropriate vector. Once in a vector, the virus may be sequenced, mutated or expressed. For example, there is provided a vector comprising the nucleic acid sequence of the genome of dengue virus comprising, but not limited to, the nucleic acid sequence of the DENV-1, DENV-2, DENV-3 and DENV-4 of SEQ ID NO: 5 to 8, respectively.

There is further provided the mutated flavivirus as described herein, wherein the non-polar amino acid that is used to replace a key amino acid in the NS5 protein of the flavivirus is an alanine, a cysteine, a glycine, an isoleucine, a leucine, a methionine, a phenylalanine, a proline, a tryptophan, a tyrosine, or a valine.

In some examples, there is provided the flavivirus as described herein, wherein the flavivirus is a tick borne encephalitis virus (TBEV) of any serotype.

The term “serotype” as used herein refers to distinct antigenic variations within a species of bacteria, virus or immune cells. In other words, it refers to a group of intimately related microorganisms distinguished by a common set of antigens. The term may also be used to refer to the set of antigens characteristic of such a group. Preferably, the nucleic acid sequence may be contained in a vector such as an infectious cDNA clone or an infectious virus particle derived from the vector. Any other suitable means of delivering the nucleic acid to a host for the purpose of vaccination known in the art may also be used. Preferably, the flavivirus is a dengue virus of any serotype or a tick borne encephalitis virus (TBEV) of any serotype.

In the context of the invention, serotype refers to distinct antigenic variations of a flavivirus such as, for example, one of the four distinct antigenic variations of the dengue virus, termed DENV-1, DENV-2, DENV-3 and DENV-4.

In one example, the non-polar amino acid as described herein may comprise, but is not limited to, an alanine, a cysteine, a glycine, an isoleucine, a leucine, a methionine, a phenylalanine, a proline, a tryptophan, a tyrosine, or a valine. In a further example, the non-polar amino acid is an Alanine. The choice of a non-polar amino acid to be used to replace a polar amino acid is determined by the structural organization of the amino acids involved in the catalytic activity of 2′-O methyltransferase, for example.

In one example, there is provided a vaccine comprising a mutated flavivirus as described herein. As used herein, the term “vaccine” is an antigenic, biological preparation used to induce immunity against a particular disease-causing pathogen. For example, as used herein, a vaccine may include a flavivirus vaccine, such as a dengue vaccine. A vaccine can comprise, but is not limited to, a protein, or part thereof, an antigen, a microorganism or a virus. Any microorganisms used as a vaccine may be inactivated prior to treatment. Vaccines can be given as a prophylaxis or as a therapeutic. The disclosure contemplates any types of vaccines known in the art. Thus, vaccination may relate to for example, administration of a vaccine to a subject in need thereof. In the context of the invention, the term “immunization” relates to the biological process that occurs within the human body after vaccination and that, as a result, confers immunity against an infectious agent.

In one example, the vaccine as used herein may comprise, but is not limited to, 1, 2, 3, 4, 5, 6, 7, 8 or more mutated flaviviruses, as disclosed herein. Each mutated flavivirus that may be administered to elicit an immune response, or to vaccinate a subject, may therefore comprise, independently, one or more mutations as described herein. The mutated flaviviruses may have the same or a different serotype.

Thus, in one aspect of the invention, there is provided a method of vaccination, comprising administration of at least one vaccine which is a mutated flavivirus, comprising at least one mutation in a nucleic acid sequence encoding for NS5 of the flavivirus sequence, whereby the at least one mutation results in inactivation of the 2′O-methyltransferase. For example, the method of vaccination, as described herein, may comprise, but is not limited to, administration of at least one, at least two, at least three, at least four, at least five, at least six or more vaccines, which are mutated viruses. Thus, the method also provides for the administration of for example, 1, 2, 3, 4, 5, 6, 7 or 8 vaccines comprising a mutated flavivirus.

In yet another example, there is provided the method, as described above, wherein the mutated flavivirus is as defined herein. In one example, there is provided the method, as described herein, wherein the mutated flavivirus is a mutated DENV-1 dengue virus having NS5 amino acid sequence of SEQ ID NO: 9, wherein Glutamic Acid 216 in the KDKE motif of NS5 of the DENV-1 dengue virus is replaced by Alanine. In one example, there is provided the method as defined herein, wherein the mutated flavivirus is a mutated DENV-1 dengue virus, wherein Lysine 61 and Glutamic Acid 216 in the KDKE motif of NS5 of the DENV-1 dengue virus are replaced by Alanine.

In one example, there is provided the method as defined herein, the method as disclosed herein, wherein the mutated flavivirus is a mutated DENV-2 dengue virus having NS5 amino acid sequence of SEQ ID NO: 10, wherein Glutamic Acid 217 in the KDKE motif of NS5 of the DENV-2 dengue virus is replaced by Alanine. In one example, there is provided the method as defined herein, wherein the mutated flavivirus is a mutated DENV-2 dengue virus, wherein Lysine 61 and Glutamic Acid 217 in the KDKE motif of NS5 of the DENV-2 dengue virus are replaced by Alanine.

In one example, there is provided the method as defined herein, wherein the mutated flavivirus is a mutated DENV-3 dengue virus having NS5 amino acid sequence of SEQ ID NO: 11, wherein Glutamic Acid 216 in the KDKE motif of NS5 of the DENV-3 dengue virus is replaced by Alanine. In one example, there is provided the method as defined herein, wherein the mutated flavivirus is a mutated DENV-3 dengue virus, wherein Lysine 61 and Glutamic Acid 216 in the KDKE motif of the NS5 of the DENV-3 dengue virus are replaced by Alanine.

In one example, there is provided the method as defined herein, wherein the mutated flavivirus is a mutated DENV-4 dengue virus having the NS5 amino acid sequence of SEQ ID NO: 12, wherein Glutamic Acid 217 in the KDKE motif of NS5 of the DENV-4 dengue virus is replaced by Alanine. In one example, there is provided the method, as defined herein, wherein the mutated flavivirus is a mutated DENV-4 dengue virus, wherein Lysine 61 and Glutamic Acid 217 in the KDKE motif of the NS5 of the DENV-4 dengue, virus are replaced by Alanine.

An “immune response” is a response of a cell of the immune system, such as a B cell, T cell, or monocyte, to a stimulus. An immune response can be a B cell response, which results in the production of specific antibodies, such as antigen-specific neutralizing antibodies. An immune response can also be a T cell response, such as a CD4+ response or a CD8+ response. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo.

A “subject” or an “individual” is a living multi-cellular vertebrate organism. In the context of this disclosure, the subject can be an experimental subject, such as a non-human animal, e.g., a mouse, a cotton rat, or a non-human primate. Alternatively, the subject can be a human subject.

In yet another example there is provided a pharmaceutical composition comprising a mutated flavivirus, as described herein, and a pharmaceutically acceptable carrier or adjuvant. In one example, the pharmaceutical composition may comprise, but is not limited to, one or two or three or four or five or six or seven or eight or more mutated flaviviruses, as described herein. The pharmaceutical compositions of the invention may contain additional substances, such as wetting or emulsifying agents, buffering agents, or adjuvants to enhance the effectiveness of the vaccines. The pharmaceutical composition may be an immunogenic composition. The pharmaceutical/immunogenic compositions disclosed herein are suitable for preventing, ameliorating and/or treating disease caused by infection with dengue virus.

The pharmaceutical composition disclosed herein may include one or more purified mutated flavivirus. The term “purification” (e.g., with respect to a pathogen or a composition containing a pathogen) refers to the process of removing components from a composition, the presence of which is not desired. Purification is a relative term, and does not require that all traces of the undesirable component be removed from the composition. In the context of vaccine production, purification includes such processes as centrifugation, dialysis, ion-exchange chromatography, and size-exclusion chromatography, affinity-purification or precipitation. Thus, the term “purified” does not require absolute purity; rather, it is intended as a relative term. Thus, for example, a purified virus preparation is one in which the virus is more enriched than it is in its generative environment, for instance within a cell, or population of cells in which it is replicated naturally, or in an artificial environment. A preparation of substantially pure viruses can be purified, such that the desired virus or viral component represents at least 50% of the total protein content of the preparation. In certain examples, a substantially pure virus will represent at least 60%, at least 70%, at least 80%, at least 85%, at least 90%, or at least 95% or more of the total protein content of the preparation.

An “isolated” biological component (such as a virus, nucleic acid molecule, protein or organelle) has been substantially separated or purified away from other biological components in the cell and/or organism in which the component occurs or, is produced. Viruses and viral components, e.g., proteins, which have been “isolated”, include viruses, and proteins, purified by standard purification methods. The term also embraces viruses and viral components (such as viral proteins) prepared by recombinant expression in a host cell.

As used herein, the term “adjuvant” is an agent that enhances the production of an antigen-specific immune response, compared to administration of the antigen in the absence of the agent. Common adjuvants include aluminum containing adjuvants, that include a suspension of minerals (or mineral salts, such as aluminum hydroxide, aluminum phosphate, aluminum hydro xyphosphate), onto which antigen is adsorbed. In the context of the present disclosure, the adjuvants are aluminum- (alum-) free adjuvants, which are formulated in the absence of any such aluminum salts. Alum-free adjuvants include oil and water emulsions, such as water-in-oil, and oil-in-water (and variants thereof, including double emulsions and reversible emulsions), liposaccharides, lipopolysaccharides, immunostimulatory nucleic acids (such as CpG oligonucleotides), liposomes, Toll-like Receptor agonists (particularly, TLR2, TLR4, TLR7/8 and TLR9 agonists), and various combinations of such components.

Adjuvants may also be included. Adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, poly IC or poly AU acids, polyarginine with or without CpG (also known in the art as IC31), certain natural substances (e.g., wax D from Mycobacterium tuberculosis, substances found in Cornyebacterium parvum, Bordetella pertussis, or members of the genus Brucella), flagellin (Toll-like receptor 5 ligand), saponins such as QS21, QS17, and QS7, monophosphoryl lipid A, in particular, 3-de-O-acylated monophosphoryl lipid A (3D-MPL), imiquimod (also known in the art as IQM), and the CCR5 inhibitor CMPD 167.

Aluminum hydroxide or phosphate (alum) is commonly used at 0.05 to 0.1% solutions in phosphate buffered saline. Other adjuvants that may be used, especially with DNA vaccines, are cholera toxin, especially CTA1-DD/ISCOMs, cytokines such as, but not limited to, IL-2, IL-4, GM-CSF, IL-12, IL-15 IGF-1, IFN-α, IFN-β, and IFN-γ, immunoregulatory proteins such as CD4OL (ADX40), and the CD1a ligand of natural killer cells (also known as CRONY or α-galactosyl ceramide), immunostimulatory fusion proteins such as IL-2 fused to the Fc fragment of immunoglobulins and co-stimulatory molecules B7.1 and B7.2, all of which may be administered either as proteins or in the form of DNA, on the same expression vectors as those encoding the flavivirus as described herein or on separate expression vectors.

In an example, the adjuvants may be lecithin is combined with an acrylic polymer (Adjuplex-LAP), lecithin coated oil droplets in an oil-in-water emulsion (Adjuplex-LE) or lecithin and acrylic polymer in an oil-in-water emulsion (Adjuplex-LAO) (Advanced BioAdjuvants (ABA)). The mutated flavivirus(es) is mixed with a suitable aluminum-free adjuvant to produce an immunogenic composition suitable for immunizing human subjects, in order to elicit high titers of virus neutralizing antibodies and protect the immunized human from disease caused by dengue virus. Typically, the mutated flavivirus(es) are formulated in a pharmaceutically acceptable carrier or excipient.

Pharmaceutically acceptable carriers and excipients are well known and can be selected by those of skill in the art. For example, the carrier or excipient can favorably include a buffer. Optionally, the carrier or excipient also contains at least one component that stabilizes solubility and/or stability. Examples of solubilizing/stabilizing agents include detergents, for example, laurel sarcosine and/or polyoxyethethylene sorbitan monooleate. Alternative solubilizing/stabilizing agents include arginine, and glass forming polyols (such as sucrose, trehalose and the like). Numerous pharmaceutically acceptable carriers and/or pharmaceutically acceptable excipients are known in the art.

Accordingly, suitable excipients and carriers can be selected by those of skill in the art to produce a formulation suitable for delivery to a subject by a selected route of administration. Suitable excipients include, without limitation: glycerol, Polyethylene glycol (PEG), Sorbitol, Trehalose, N-lauroylsarcosine sodium salt, L-proline, Non detergent sulfobetaine, Guanidine hydrochloride, Urea, Trimethylamine oxide, KCl, Ca2+, Mg2+, Mn2+, Zn2+ and other divalent cation related salts, Dithiothreitol, Dithioerytrol, and β-mercaptoethanol. Other excipients can be detergents (including: polyoxyethethylene sorbitan monooleate, Triton X-00, NP-40, Empigen BB, Octylglucoside, Lauroyl maltoside, Zwittergent 3-08, Zwittergent 3-0, Zwittergent 3-2, Zwittergent 3-4, Zwittergent 3-6, CHAPS, Sodium deoxycholate, Sodium dodecyl sulphate, Cetyltrimethylammonium bromide).

When provided prophylactically, the pharmaceutical compositions, as disclosed herein, may be ideally administered to a subject in advance of infection, such as flaviviruses infection, or therapeutic administration upon evidence of flaviviruses infection, or in advance of any symptom due to, for example, Dengue fever, especially in high-risk subjects. The prophylactic administration of the immunogenic compositions may serve to provide protective immunity of a subject against flavivirus infection, such as dengue virus infection or therapeutic administration to prevent or attenuate the progression of dengue fever in a subject already infected with dengue virus. When provided therapeutically, the pharmaceutical compositions may serve to ameliorate and treat flavivirus infection, symptoms and are advantageously used as soon after infection as possible, preferably before appearance of any symptoms of dengue fever but may also be used at (or after) the onset of the disease symptoms.

The pharmaceutical compositions may be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods. Further, delivery of nucleic acids to animal tissue has been achieved by cationic liposomes, direct injection of naked nucleic acids into animal muscle tissue, or intradermal injection of nucleic acids using “gene gun” technology. Alternatively, delivery routes may be oral, intranasal or by any other suitable route. Delivery may also be accomplished via a mucosal surface such as the anal, vaginal or oral mucosa. Immunization schedules (or regimens) are well known for animals (including humans) and may be readily determined for the particular subject and immunogenic composition. Hence, the immunogens may be administered one or more times to the subject. Preferably, there is a set time interval between separate administrations of the immunogenic composition. While this interval varies for every subject, typically it ranges from 10 days to several weeks, and is often 2, 4, 6 or 8 weeks. For humans, the interval is typically from 2 to 6 weeks. The immunization regimes typically have from 1 to 6 administrations of the immunogenic composition, but may have as few as one or two or four. The methods of inducing an immune response may also include administration of an adjuvant with the immunogens. In some instances, annual, biannual or other long interval (5-10 years) booster immunization may supplement the initial immunization protocol.

The pharmaceutical compositions may be administered using any suitable delivery method including, but not limited to, buccal, sublingual, rectal, topical, nasal, intramuscular, intradermal, subcutaneous, intravenous, intradermal, mucosal, and topical delivery. Such techniques are well known to those of skill in the art. Thus, there is provided the method as disclosed herein, wherein administration may comprise, but is not limited to, buccal, sublingual, rectal, topical, nasal, intramuscular, intradermal and subcutaneous administration. More specific examples of delivery methods are intramuscular injection, intradermal injection, and subcutaneous injection. However, delivery need not be limited to injection methods. In one example there is provided the method as described above, wherein administration comprises, but is not limited to, buccal, sublingual, rectal, topical, nasal, intramuscular, intradermal and subcutaneous delivery. In one example below, the vaccine is injected intraperiteonally.

In one example, the administration as disclosed herein may comprise, but is not limited, to one, two, three, four, five, six, seven, eight or more mutated flaviviruses, wherein the mutated flaviviruses may be different viruses, such as dengue virus or tick borne encephalitis virus, or may be the same flaviviruses having the same or different serotypes. The administration of the mutated flaviviruses, as described above, may improve the immune response and protection against various strains or serotypes of flaviviruses. For example, the administration for eliciting an immune response or vaccination may comprise, but is not limited to, dengue viruses of each one of the four serotypes, each serotypes comprising at least one mutation. Thus, it is understood that administration may comprise, for example, dengue viruses, having one, two, three, four, five, six, seven, eight or more different nucleic acid sequences, as described herein.

In one example there is provided a method of preventing a disease caused by dengue virus by administering to a subject a vaccine as described herein. For example, the pharmaceutical compositions or vaccine can include a single strain of dengue virus (i.e., a monovalent composition), or they can contain more than one strain of dengue virus (i.e., a multivalent composition). For example, the vaccine may comprise, but is not limited, to 1, 2, 3, 4, 5, 6, 7, 8 or more mutated dengue viruses, as disclosed herein. Typically, a multivalent composition contains strains selected from different serotypes. Because there are four serotypes of dengue virus, which can cause disease and because cross-reactive non-neutralizing antibodies are predisposing to more severe forms of dengue disease, one representative of each serotype can be selected for inclusion into the final vaccine in order to guarantee protection against disease from any of the four serotypes. Thus, in one example, the pharmaceutical composition is a tetravalent composition that includes strains selected from each of the four serotypes of dengue virus.

The viruses used as antigens can be selected from essentially any strain (or strains) of flavivirus, such as dengue virus. For example, a flavivirus strain can be selected for each serotype, which is chosen based on its conformity to a defined (e.g., consensus) sequence for the serotype, such as a DENV-1 consensus sequence, a DENV-2 consensus sequence, a DENV-3 consensus sequence, or a DENV-4 consensus sequence. Such a virus can be naturally occurring or synthetic. Alternatively, a virus strain can be selected to correlate with a strain prevalent in the area or population, in which the vaccine is intended to be administered. Another option is to select strains for each serotype as a matter of convenience based on availability or prior experience.

In the context of a purified mutated flavivirus vaccine, either virulent or attenuated strains can be used. Typically, virulent strains propagate to higher titers in host cells, facilitating production at commercial scale. However, virulent strains require special care in handling to prevent infection of personnel involved in manufacturing. Advantageously, attenuated strains require fewer handling precautions, but can be difficult to produce. Exemplary attenuated strains suitable for use in the context of a pharmaceutical composition containing an inactivated dengue virus and an aluminum-free adjuvant. Thus, the strain(s) selected are typically chosen from among the numerous strains available to replicate in cells that are suitable for production of materials intended for human use (e.g., cells that are certified free of pathogens). For example, strains can be screened to identify those viruses that grow to the highest titers, for example from a titer of at least about 1×10² pfu/ml, at least about 5×10² pfu/ml, at least about 1×10³ pfu/ml, at least about 5×10³ pfu/ml, at least about 1×10⁴ pfu/ml, at least about 5×10⁴ pfu/ml, at least about 1×10⁵ pfu/ml, at least about 1×10⁶ pfu/ml, at least about 1×10⁷ pfu/ml or more in the cell line(s) of choice; (ii) selecting those strains of dengue virus which grow to the highest titers in the cell line(s) of choice; and (iii) further adapting those selected strains for enhanced growth by additional passage from one to several times in the cell line(s) of choice. The selected flaviviruses (for example, chosen from the four serotypes of dengue viruses) can be further adapted to grow to high titers by additional cell culture passages or by genetic manipulation to make high-titer master and production seed lots.

Suitable cell lines for propagating dengue virus include mammalian cells, such as Vero cells, AGMK cells, BHK-21 cells, COS-1 or COS-7 cells, MDCK cells, CV-1 cells, LLC-MK2 cells, primary cell lines such as fetal Rhesus lung (FRhL-2) cells, BSC-1 cells, and MRC-5 cells, or human diploid fibroblasts, as well as avian cells, chicken or duck embryo derived cell lines, e.g., AGE1 cells, and primary, chicken embryo fibroblasts, and mosquito cell lines, such as C6/36. Preferably, the chosen cell(s) are adapted to grow in the absence of serum or serum-derived proteins, and can maintain dengue virus replication at high titers under serum-free (and/or protein-free) growth conditions.

To propagate virus in cell culture, the selected flavivirus virus strain is used to infect the host cell (for example, selected from among the suitable cell types listed above). After virus adsorption, the cultures are fed with medium capable of supporting growth of the cells. Preferably, the medium does not contain serum, or serum-derived proteins, or other animal-derived proteins, or serum-free media can be used to replace serum-containing media during production. Numerous formulations of serum-free medium are available commercially.

The host cells are maintained in culture for several days until the desired virus titer is achieved. Optionally, the cells are maintained in a continuous perfusion system from which virus can be intermittently or continuously obtained over the course of several days or more. Under non-continuous culture conditions, a virus titer of at least about 10⁶ to 10⁷ pfu/ml by 3-7 days post-infection is desirable. In some host cells, the titer remains high for several days, and virus can be recovered at multiple time points to maximize yield. For example, virus can be harvested from these cultures daily, from about 3 to about 13 days post-infection by collecting the supernatants and re-feeding the cells. Optionally, the supernatants can be pooled prior to additional processing. In other host cells, virus can be grown to a higher titer, but over a shorter period of time. In such a case, the virus can be harvested at peak titer as determined empirically. In the examples below, there is provided examples of production of the flavivirus as described herein.

In a further example, there is provided the method as described above, wherein an immunization is obtained by one time administration of the vaccine. In yet another example, there is provided the method, as described herein, wherein immunization is obtained by administration of a priming dose followed by at least one booster dose. As described herein, the term “prime vaccination dose” is used to describe the first and initial dose of a vaccine given to a subject in order to induce an immune response against an infectious agent. The term “booster” dose, as defined herein, describes any and all subsequent doses of the same vaccine given to the individual in order to further enhance immunity against the infectious agent.

Typically, vaccines are prepared as injectables, either as liquid solutions or suspensions; solid form suitable for solution in, or suspension in, liquid prior to injection may also be prepared. Although the composition can be administered by a variety of different routes, most commonly, the immunogenic compositions are delivered by an intramuscular, subcutaneous or intradermal route of administration. Generally, the vaccine may be administered subcutaneously, intradermally, or intramuscularly in a dose effective for the production of neutralizing antibody and protection. The vaccines are administered in a manner compatible with the dosage formulation, and in such amount as will be prophylactically and/or therapeutically effective. The quantity to be administered, which is generally in the range of 0.05-100 μg of each strain of flavivirus per dose, depends on the subject to be treated, capacity of the subject's immune system to synthesize antibodies, and the degree of protection desired. Precise amounts of the vaccine to be administered may depend on the judgment of the practitioner and may be peculiar to each subject.

The vaccine may be given in a single dose schedule, or preferably a multiple dose schedule in which a primary course of vaccination may be with 1, 2, 3, 4, 5, 6, 7, 8, 9 or 10 separate doses, followed by other doses given at subsequent time intervals required to maintain and or reinforce the immune response, for example, at 1, 2, 3 or 4 months for a second dose, and if needed, a subsequent dose(s) after 2, 3, 4, 5, 6, 7, 8, 9, 10, 11, 12, 13, 14, 15, 16, 17, 18, 19, 20, 21, 22 or 23 months or 2, 3, 4, 5, 6, 7, 8 or 9 years. The dosage regimen will also, at least in part, be determined by the need of the individual and be dependent upon the judgment of the practitioner. Examples of suitable vaccination schedules include: a first dose, followed by a second dose between 7 days and 6 months, (for example, the second dose may be 7 days or 14 days or 3, 6 or 9 weeks or 2, 3, 4, 5 or 6 months after the initial vaccination) and an optional third dose between 1 month and two years post-initial vaccination, (for example, 1, 2, 3, 4, 5, 6, 7, 8, 9, 10, 11, 12, 14, 16, 18, 20, 22 or 24 months post-initial vaccination) or other schedules sufficient to elicit titers of virus-neutralizing antibodies expected to confer protective immunity, for example selected to correspond to an established pediatric vaccine schedule. The generation of protective immunity against dengue virus with an inactivated virus vaccine may reasonably be expected after a primary course of vaccination consisting of 1 or 2 or 3 inoculations. These could be supplemented by boosters at intervals (e.g., every two years) designed to maintain a satisfactory level of protective immunity. In some examples, the vaccine as described herein may provide protection for, at least one, at least two, at least three, at least four, at least five, at least 10 or more years of protective immunity against the flavivirus of interest. In one example, protective immunity may be provided for a lifetime after a single injection.

In another example, there is provided a prime/boost protocol, wherein a first vaccination occurs at time point 0, followed by a second vaccination at any time point between about 2 or 3 months to about 12 months after the first vaccination. For example, the second vaccination may be about 3 months, or about 4 months, or about 5 months, or about 6 months, or about 7 months, or about 8 months, or about 9 months, or about 10 months, or about 11 months or about 12 months. The second vaccination is followed by a booster vaccination at intervals of about two to about ten years to maintain protective immunity. In some examples, the dosage per vaccination may comprise, but is not limited to, any dosage between about 10² pfu, or about 5×10² pfu, or about 10³ pfu, or about 5×10³ pfu, or about 10⁴ pfu, or about 5×10⁴ pfu, or about 10⁵ pfu, or about 5×10⁵ pfu, or about 10⁶ pfu, or more of attenuated virus per serotype.

The present disclosure relates to mutated flaviviruses as vectors, however, other vectors may be contemplated in other embodiments such as, but not limited to, prime boost administration, which may comprise administration of a mutated flavivirus vector in combination with another recombinant vector expressing vaccine antigens derived from one or more flavivirus, such as dengue. Alternative vaccine boosting strategies may include, but are not limited to, protein subunit vaccines, toxoid vaccines, conjugate vaccines, DNA vaccines, virus-like particle vaccines, as well as live attenuated or inactivated vectored vaccines.

When the aim is to deliver antigens of the invention in vivo in a subject, for example, in order to generate an immune response against a mutated flavivirus, and/or an antigen and/or protective immunity against a flavivirus, expression vectors that are suitable for expression in that subject, and that are safe for use in vivo, should be chosen. For example, it may be desirable to express the antigens, such as the vaccine antigen, in a laboratory animal, such as for pre-clinical testing of the flavivirus immunogenic compositions and vaccines, as disclosed herein. In other examples, it will be desirable to express the antigens of the invention in human subjects, such as in clinical trials and for actual clinical use of the immunogenic compositions and vaccine of the invention. Any vectors that are suitable for such uses may be employed, and it is well within the capabilities of the skilled artisan to select a suitable vector. In some embodiments it may be preferred that the vectors used for these in vivo applications are attenuated. For example, if plasmid vectors are used, preferably they will lack an origin of replication that functions in the subject, so as to enhance safety for in vivo use in the subject. If viral vectors are used, preferably they are attenuated or replication-defective in the subject, again, so as to enhance safety for in vivo use in the subject.

In some examples recombinant enveloped viruses may be used as vectors, however, other vectors may be contemplated in other examples such as, but not limited to, prime-boost administration, which may comprise administration of a recombinant envelope virus vector in combination with another recombinant vector expressing one or more flavivirus epitopes.

The nucleotide sequences and vectors as disclosed herein may be delivered to cells, for example, if the aim is to generate viral particles containing the desired antigenic protein. Suitable transfection, transformation, or gene delivery methods may be used as part of this objective. Such methods are well known by those skilled in the art, and one of skill in the art would readily be able to select a suitable method, depending on the nature of the nucleotide sequences, vectors, and cell types used. For example, transfection, transformation, microinjection, infection, electroporation, lipofection, or liposome-mediated delivery could be used. Generation of the viral particles containing the desired antigens may be carried out in any suitable type of host cells, such as bacterial cells, yeast, insect cells, and mammalian cells. The antigens of the invention may also be expressed including using in vitro transcription/translation systems. All of such methods are well known by those skilled in the art, and one of skill in the art would readily be able to select a suitable method depending on the nature of the nucleotide sequences, vectors, and cell types used.

Thus, in one example, there is provided the method, as described herein, wherein the vaccination comprises administration of a further vaccine, different from the mutated flavivirus. In another example, there is provided the method as described herein, wherein the further vaccine comprises a vector selected from the group consisting of herpesvirus, poxvirus, hepadnavirus, togavirus, coronavirus, hepatitis D virus, orthomyxovirus, paramyxovirus, rhabdovirus, bunyavirus, measles, canine distemper virus and filovirus.

As indicated above, the use of other recombinant viruses may be envisaged during the booster vaccination. VSV is a practical, safe, and immunogenic vector for conducting animal studies, and an attractive candidate for developing vaccines for use in humans. VSV is a member of the Rhabdoviridae family of enveloped viruses containing a non-segmented, negative-sense RNA genome. The genome is composed of 5 genes arranged sequentially 3′-N-P-M-G-L-5′, each encoding a polypeptide found in mature virions. Notably, the surface glycoprotein G is a transmembrane polypeptide that is present in the viral envelope as a homotrimer, and like Env, it mediates cell attachment and infection.

In some examples, Canine Distemper Viruses (CDVs) may be contemplated by the present disclosure. In other examples, measles may be contemplated by the present disclosure.

Other envelope viruses are also contemplated, such as a herpesvirus, poxvirus, hepadnavirus, togavirus, coronavirus, hepatitis D virus, orthomyxovirus, paramyxovirus, rhabdovirus, bunyavirus or a filovirus.

In one example, there is provided the method described herein, wherein vaccination and/or immunization is for preventing a disease, wherein the disease comprises, but is not limited to, dengue fever (DF), dengue hemorrhagic fever (DHF), dengue shock syndrome (DSS), dengue fever (DF) together with dengue shock syndrome (DSS), dengue hemorrhagic fever (DHF) together with dengue shock syndrome (DSS). In another example there is provided the method as described above, wherein the disease is selected from the group consisting of dengue fever (DF), dengue hemorrhagic fever (DHF), dengue shock syndrome (DSS), dengue fever (DF) together with dengue shock syndrome (DSS), dengue hemorrhagic fever (DHF) together with dengue shock syndrome (DSS).

When the flavivirus is used to vaccinate a subject, it is understood that different regimens may be used. As described herein, there are typically three doses based on the amount of virus in the dose. Since the exact number of virus in a dose is difficult to estimate, the skilled person in the art would often refer to the arbitrary plaque forming units. As such, in the context of the present disclosure, the term “low dose” is used for doses containing between about 1×10² pfu to about 1×10⁴ pfu. The term “medium dose” is used for between about 1×10⁴ pfu to about 1×10⁵ pfu, whereas the term “high dose” is used for doses comprising between about 1×10⁵ pfu and about 1×10⁶ pfu. In one example, a low dose is about 1×10³ pfu, a medium dose is about 1×10⁴ pfu and a high dose is about 1×10⁵ pfu. For example, there is provided the method, as described herein, wherein the vaccine is to be administered at a dose comprising, but not limited to, about 1×10² pfu, or about 5×10² pfu, about 1×10³ pfu, or about 5×10³ pfu, or about 1×10⁴ pfu, or about 5×10⁴ pfu, or about 1×10⁵ pfu, or about 5×10⁵ pfu, or about 1×10⁶ pfu. In a further example, there is provided the method as described herein, wherein the vaccine is to be administered at a dose of between about 1×10³ pfu to 1×10⁵ pfu. In a further example, there is provided the method as described herein, wherein the vaccine is to be administered at a dose of about 1×10³ pfu. Some examples of the doses and administration, as disclosed herein, are provided in some of the non-limiting examples below.

A method of preventing a flavivirus infection is described, comprising administering to an individual an attenuated flavivirus according to any one of claims as at least one injection. In one example, at least one injection may be a single injection. In another embodiment, at least one injection may be multiple injections of two or more such as those known in the art. In one example, there is provided the method of using the mutated flavivirus, as described herein, for vaccination against dengue infection from any serotype. Hence, the mutated flavivirus used for vaccination may include a combination of 2, 3, 4, 5, 6, 7, 8 or more dengue viruses with the same or different phenotypes and with the same (or equivalent) or different mutations, for example, in the coding sequence of the NS5 protein, such as the coding nucleic acid sequence of the 2′-O MTase.

In a further example, there is provided a method of using the mutated flavivirus, as described herein, in a combination of any number of different flavivirus genotypes for vaccination against dengue infection from any serotype. For example, the method may include, but is not limited, to 1, 2, 3, 4, 5, 6, 7, 8 or more mutated flaviviruses with same or different serotypes and/or with same or different mutations that inactivate the flaviviruses. In yet another example, there is provided a method of manufacturing a mutated flavivirus, as described herein, using a reverse genetics system. Methods of manufacturing flavivirus are known to the person skilled in the art. In some examples, the flavivirus, as described herein, may be purified using methods, such as with differential centrifugation, with density gradient purification, with precipitation, with size exclusion or other chromatographic methods, with size exclusion filtration. These methods, as described herein, may be used sequentially in any possible order.

The pharmaceutical compositions of the invention may be administered alone, or may be co-administered, or sequentially administered, with other flavivirus immunogens, vaccines and/or flavivirus pharmaceuticals compositions, e.g., with “other” immunological, antigenic or vaccine or therapeutic compositions thereby providing multivalent or “cocktail” or combination compositions of the invention and methods of employing them. Again, the ingredients and manner (sequential or co-administration) of administration, as well as dosages may be determined by taking into consideration such factors as the age, sex, weight, species and condition of the particular subject, and the route of administration.

When used in combination, the other flavivirus immunogens may be administered at the same time, or at different times, as part of an overall vaccination regime, e.g., as part of a prime-boost regimen or other vaccination protocol.

A pharmaceutical composition may comprise a mutated flavivirus as described herein; a carrier wherein the carrier is optionally selected from carrier moieties useful in vaccination (e.g. vesicles such as liposomes) and carrier moieties useful for diagnostic purposes (e.g. particles of silica, latex, or gold; membranes of nylon, PVDF, nitrocellulose, or paper etc.); a pharmaceutically acceptable carrier or adjuvant (e.g. alum, Montanide, squalene, QS21, MF59 or CpG).

In some examples, there is provided virus particles derived from the above clones. In another example, there is provided the use of such particles in pharmaceutical compositions for vaccination against Dengue infection and/or disease. In yet another example, there is provided the use of clones from Dengue serotype 1, 2, 3 and 4 by themselves or in combination, with or without adjuvants, as single injection or in prime-boost vaccination protocols.

In the following, further examples are provided.

An attenuated flavivirus for vaccination is described comprising a nucleic acid sequence, wherein NS5 of the flavivirus sequence has at least one mutation resulting in the expression of an amino acid, whereby a polar amino acid is replaced with a non-polar amino acid at Lysine 61, Lysine 181 or Glutamic acid 217 or equivalent respective amino acid positions in a KDKE motif of a 2′O-methyltransferase of NS5 of the flavivirus. An amino acid is an organic compound consisting of an amine (—NH₂), a carboxylic acid (—COOH) functional group and a side-chain specific to each amino acid. This includes, but is not limited to, all proteogenic (amino acids encoded by the genetic code), all non-proteogenic (artificial amino acids not encoded by the genetic code), all standard and all non-standard amino acids. A polar amino acid is an amino acid, wherein the distribution of electrons across the molecule is uneven, resulting in an electric dipole, due to the differing electron negativities of the amino acid side chains. A non-polar amino acid is an amino acid, wherein the electrons are evenly distributed over the whole molecule. A mutation is a modification of the genome or part of a nucleic acid sequence of any biological organism, virus or extrachromosomal genetic element. This mutation can be induced artificially using, but not limited to, chemicals and radiation, but can also occur spontaneously during nucleic acid replication in cell division.

Alternatively, an attenuated flavivirus for vaccination is described comprising a nucleic acid sequence, wherein NS5 of the flavivirus sequence at least one mutation resulting in the expression of an amino acid whereby a polar amino acid is replaced with a non-polar amino acid at Lysine 14, Lysine 29, Isoleucine 147, Glutamic acid 35 or Tryptophan 87 or equivalent respective amino acids in the GTP-pocket, SAM-pocket or RNA binding site of NS5 of the flavivirus.

In one example, the flavivirus is a dengue virus 2 ribonucleic acid sequence. In another example, the flavivirus is a dengue virus 1 ribonucleic acid sequence. In another example, the flavivirus is a dengue virus 3 ribonucleic acid sequence. In another example, the flavivirus is a dengue virus 4 ribonucleic acid sequence. Preferably, the attenuated virus further comprises a nucleic acid sequence of at least two dengue virus strains, a second or subsequent strain comprising, but not limited to a dengue virus 1, a dengue virus 2, a dengue virus 3 and a dengue virus 4. In one example, there is provided a method of using the attenuated flavivirus in any combination of serotypes 1 to 4 and in any combination of different genotypes within the groups of serotypes 1 to 4 of this example may be used for vaccination against dengue infection from any DENV serotype. The vaccine may be administered concomitantly or subsequently. Preferably, the non-polar amino acid is an Alanine. Ribonucleic acids are biomolecules that play an important role in the regulation, coding, decoding and expression of genes. Each ribonucleic acid consists of a nucleotide, either adenine (A), cytosine (C), guanine (G) or uracil (U), and a ribose sugar. A ribonucleic acid sequence comprises of a chain of these nucleic acids, resulting in a sugar-phosphate backbone.

NS5 of the flavivirus sequence may have at least two mutations, resulting in the expression of an amino acid, whereby a polar amino acid is replaced with a non-polar amino at Lysine 61, Lysine 181 or Glutamic acid 217 or equivalent respective amino acid positions in the KDKE motif; of a 2′O methyltransferase of NS5 of the flavivirus. Alternatively, NS5 of the flavivirus sequence may have at least two mutations, resulting in the expression of an amino acid, whereby a polar amino acid is replaced with a non-polar amino acid at Lysine 14, Lysine 29, Isoleucine 147, Glutamic acid 35 or Tryptophan 87 or equivalent respective amino acids in the GTP-pocket, SAM-pocket or RNA binding site of NS5 of the flavivirus.

In one example, the flavivirus is a tick borne encephalitis virus (TBEV) of any serotype. A method of using the attenuated flavivirus of this example may be used for vaccination against TBEV infection from any TBEV serotype. The vaccine may be administered concomitantly or subsequently.

A vaccine may comprise a mutation in any of the key amino acid KDKE of a 2′O-methyltransferase, GTP-pocket, SAM-pocket or RNA binding site of the 2′O-methyltransferase NS5 of the flavivirus.

In one example, the vaccine is suitable for protection against a dengue virus serotype 2. In one example, the vaccine is suitable for protection against a dengue virus serotype 1. In one example, the vaccine is suitable for protection against a dengue virus serotype 3. In one example, the vaccine is suitable for protection against a dengue virus serotype 4. In one example, the vaccine is suitable for protection against one or more serotypes and genotypes of a dengue virus chosen from the group of serotypes, 1, 2, 3, and 4.

In one example, the vaccine is against a tick borne encephalitis virus (TBEV).

Preferably, the vaccine further comprises at least 2 mutations in the KDKE domain of a 2′O methyltransferase, the GTP-pocket, SAM-pocket or RNA-binding site of NS5 of the flavivirus.

One example of the technology consists of the following features: an attenuated dengue vaccine comprising a nucleic acid sequence having at least 95% homology with a dengue virus 2 and an attenuated dengue vaccine comprising a nucleic acid sequence having at least 95% homology with a dengue virus 1 ribonucleic acid sequence, wherein at NS5 of the dengue virus sequence at least one mutation resulting in the expression of an amino acid, whereby a polar amino acid is replaced with a non-polar amino acid at Lysine 61, Lysine 181 or Glutamic acid 217 or equivalent respective amino acid positions in the KDKE motif; or Lysine 14, Lysine 29, Isoleucine 147, Glutamic acid 35 or Tryptophan 87 or equivalent respective amino acids in the GTP-pocket, SAM-pocket or RNA binding site of the 2′O-methyltransferase of NS5 of any flavivirus. Preferably, the non-polar amino acid is an Alanine.

Preferably, there are at least two mutations listed above, in the vector. Preferably, the vector comprises the nucleic acid sequence of at least 2 dengue virus strains, a third or subsequent strain comprising, but not limited, to dengue virus 3 and a dengue virus 4. Similarly, in one example, there is provided a method of using the vaccine, as described herein. In one example, there is provided the introduction of at least two mutations listed above into a vector having a nucleic acid sequence having at least 95% homology with a tick borne encephalitis virus (TBEV) of any serotype.

Mutations reduce 2′O-methylation and not N-7 methylation, resulting in an attenuated virus for use as a vaccine. In one example, there is provided the use of a mutation in any of the key amino acids in the KDKE motif, the GTP-pocket, SAM-pocket or the RNA-binding site of the 2′O methyltransferase to inactivate 2′O methylation. In a further example, there is provided a vaccine comprising a mutation in a dengue virus serotype 2. In yet another example, there is provided a vaccine comprising a mutation in a dengue virus serotype 1. In one example, there is also provided a vaccine comprising at least 2 mutations in the KDKE domain, the GTP-pocket, SAM-pocket or the RNA-binding site.

In one example, there is provided an attenuated virus for use as a vaccine by mutating the domain of KDKE, the GTP-pocket, SAM-pocket or the RNA-binding site of a DENV-2 or a DENV-1 at 2′O methyltransferase. Surprisingly, the attenuated divalent DENV-1/DENV-2 vaccine effectively protects against DENV-1 as well as DENV-2 infection. This is unexpected, as competition effects between strains have been reported.

In one example, there is provided a pharmaceutical composition comprising an attenuated flavivirus, as described herein, a carrier, wherein the carrier is optionally selected from carrier moieties useful in vaccination (e.g. vesicles such as liposomes) and carrier moieties useful for diagnostic purposes (e.g. particles of silica, latex, or gold; membranes of nylon, PVDF, nitrocellulose, or paper etc.), and a pharmaceutically acceptable carrier or adjuvant (e.g. alum, Montanide, squalene, QS21, MF59 or CpG)

In one example, there a method of preventing a flavivirus infection is described by administering to an individual an attenuated flavivirus according to any one of claims as at least one injection. In one example, at least one injection may be a single injection. In another example, at least one injection may be multiple injections of two or more, such as those known in the art as prime boost protocols.

A prime vaccination dose is the term used to describe the first and initial dose of a vaccine given to a subject in order to induce an immune response against an infectious agent. The term “booster” dose is used to describe any and all subsequent doses of the same vaccine given to the individual to in order to further enhance immunity against the infectious agent.

In one example, there is provided the use of such particles in pharmaceutical compositions for vaccination against Dengue infection and/or disease. In another example, there is provided the use of clones from Dengue serotype 1, 2, 3 and 4 by themselves or in combination, with or without adjuvants, as single injection or in prime-boost vaccination protocols.

Although the present invention and its advantages have been described in detail, it should be understood that various changes, substitutions and alterations may be made herein without departing from the spirit and scope of the invention as defined in the appended claims.

### Experimental Section

Viruses defective in 2′-O methylation are attenuated in vitro and in vivo. We constructed two mutant MTases containing Ala-substitutions at the K-D-K-E tetrad: one with a single E217A mutation and another with double K61A+E217A mutations. Here we demonstrate that these Dengue virus mutants lack 2′O-MTase activity and are highly sensitive to type I interferon; these virus mutants are attenuated in mice and rhesus monkeys and elicit a strong adaptive immune response. AG129 mice vaccinated once with a divalent mutant Dengue 1/Dengue 2 combination produced IgG titers of between 1:10,000 to 1:20,000, five days after challenge. No interference between the two serotypes of dengue MTase mutant vaccines could be observed in terms of viremia and antibody titers generated when two strains were given at the same time and in equal concentrations. Monkeys vaccinated with a single dose of Dengue 2 MTase mutant virus showed 100% seroconversion even when a dose as low as 1000 plaque forming units was administrated. Animals were fully protected against homologous challenge. These results clearly demonstrate the potential of 2′-O MTase Dengue mutants as safe, rationally designed live attenuated vaccine candidates.

The fact that DENV 2′-O MTase mutants grow in tissue culture to titers comparable to wildtype (wt) virus and that related viruses with 2′-O MTase mutations are attenuated in their natural host, makes these mutants promising vaccine candidates.

Infectious virus clones of dengue virus type 1, 2, 3, 4 containing mutations in the 2′O-Methyltransferase gene that result in loss of 2′-O-methyltransferase activity. Mutations include but are not limited to: E217A, K61A, K14A, K29A, I147A, E35A, W87A for the mutations identified as abrogating the 2′-O methyltransferase activity while maintaining N-7-methyltransferase activity necessary for virus viability.

### Example 1

### N7 and 2′-O Methylation Activities of Wt and Mutant DENV-1 and DENV-2

Flaviviruses are positive-sense, single-stranded RNA viruses replicating in the cytoplasm. The cytoplasm-replicating viruses have evolved N7- and 2′-O-methyltransferases (MTase) to methylate their viral mRNA 5′ cap structures. It had been previously shown for West Nile virus (WNV) and DENV-1 virus that mutation of the Asp of the tetrad K-D-K-E completely abolished N7 and 2′-O MTase activities, and was lethal for viral replication; mutations of the other three residues of the tetrad abolished 2′-O methylation (with a slight decrease in N7 methylation), and led to attenuated viruses. Since there are four serotypes of DENV, the above-mentioned MTase mutation was introduced into DENV-2 virus for proof of concept that the same approach was feasible with more than one serotype.

A wild-type (WT) recombinant MTase, representing the N-terminal 296 amino acids of the DENV-2 NS5 (strain TSV01), was cloned and expressed. Two mutant MTases containing Ala-substitutions at the K-D-K-E tetrad (FIG. 1A) were prepared: one with a single E217A mutation and another with double K61A+E217A mutations. The mutant enzymes retained 95% and 77% of the WT N7 methylation activity, respectively; neither mutant exhibited any 2′-O methylation activity (FIG. 1B). BHK-21 cells transfected with equal amounts of WT and mutant (E217A and K61A+E217A) genome length RNAs of DENV-2 virus generated equivalent number of viral E protein-expressing cells (FIG. 1C). Both WT and mutant RNAs produced infectious viruses (passage 0) with similar plaque morphologies (FIG. 1D). The replication of mutant viruses was attenuated in mammalian Vero and mosquito C3/36 cells (FIG. 1E). Continuous culturing of the mutant viruses on Vero cells or HWK-293 cells expressing DC-SIGN (HEK-DC-SIGN) for ten rounds (3-4 days per round) did not change their plaque morphologies (FIG. 1D and data not shown). The expression of DC-SIGN facilitates DENV infection.

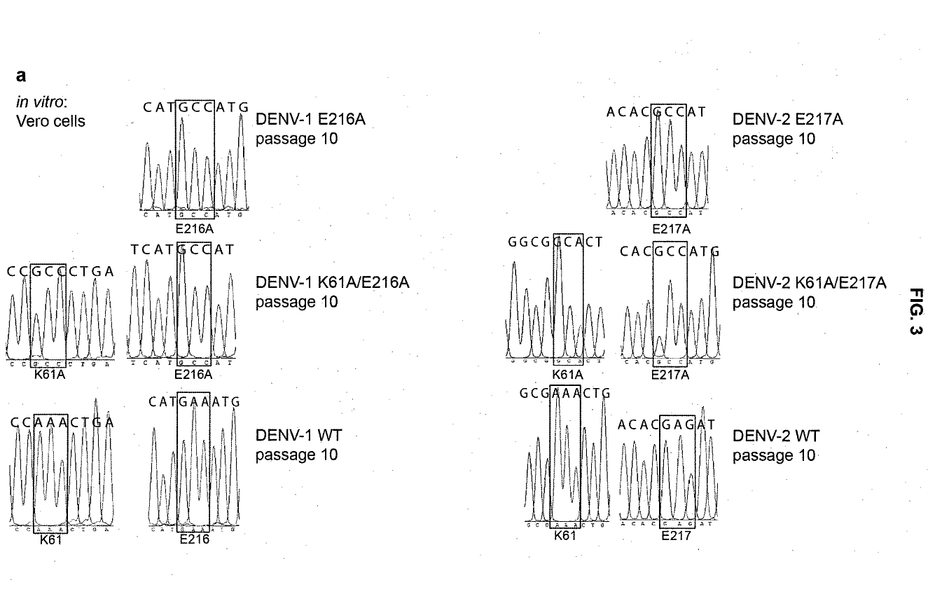

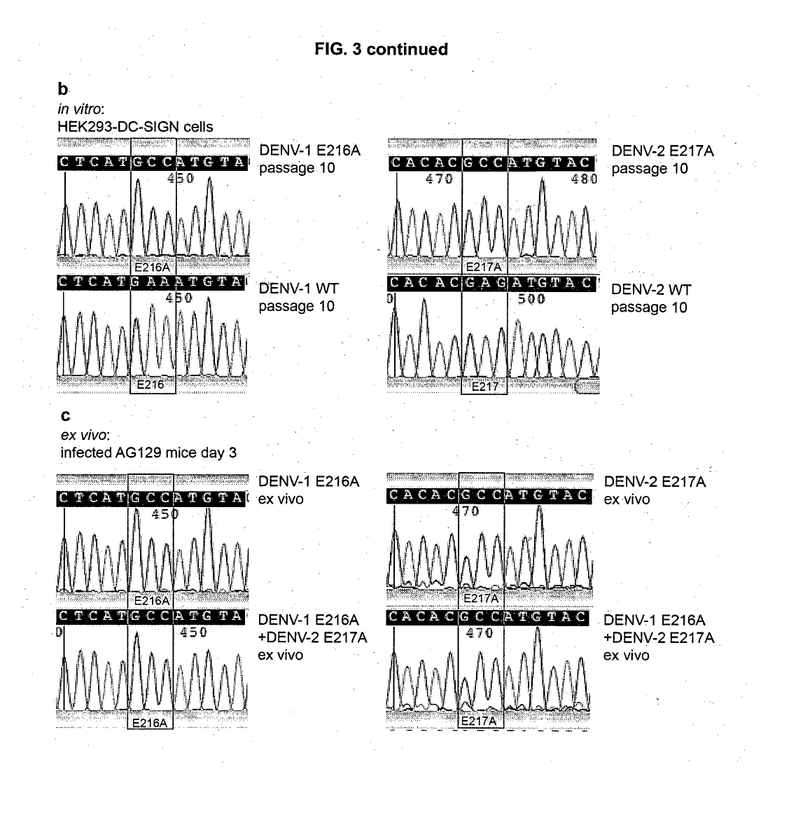

Sequencing of the passage 0 and 10 viruses from both Vero and HEK-DC-SIGN cells showed that the engineered mutations were retained (FIG. 3). Similar results were obtained for DENV-1 containing the E216A (E216 in DENV-1 MTase is equivalent to E217 in DENV-2 MTase) or K61A+E216A mutation in MTase (FIG. 2). Collectively, the results demonstrate that the 2′-O MTase mutant DENV-1 and -2 are slightly attenuated, but stable in cell culture.

The above-mentioned double mutations were also performed in DENV-3 and -4 viruses. The following table 1 shows which WT strain was used in the generation of each double mutant virus.

TABLE 1
DENV wildtype strains and mutations introduced for attenuation

| | | | | Genbank |
|---|---|---|---|---|
| | Mutation 1 | Mutation 2 | Wildtype | number |
| DENV-1 | E216A | K61A | DENV-1 Westpac | U88535.1 |
| DENV-2 | E217A | K61A | DENV-2 TSV01 | AY037116.1 |
| DENV-3 | E216A | K61A | D3MY05-34640 | FN429918 |
| DENV-4 | E217A | K61A | D4MY01-22713 | FN429920 |

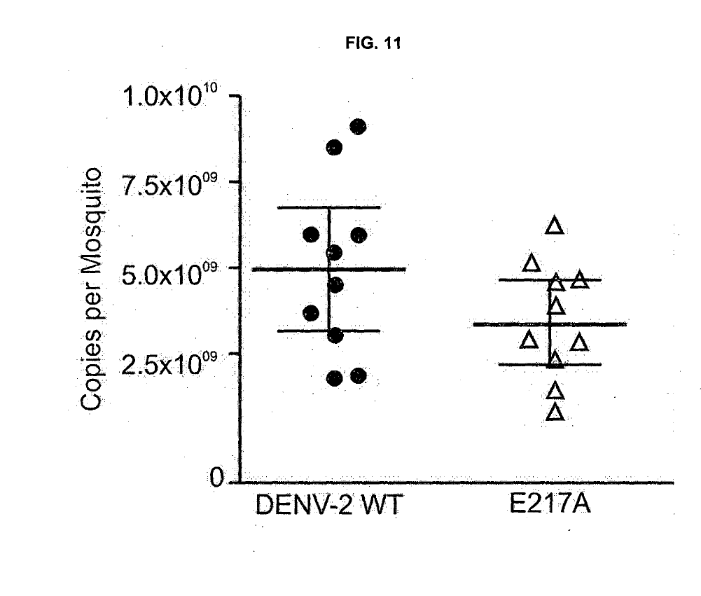

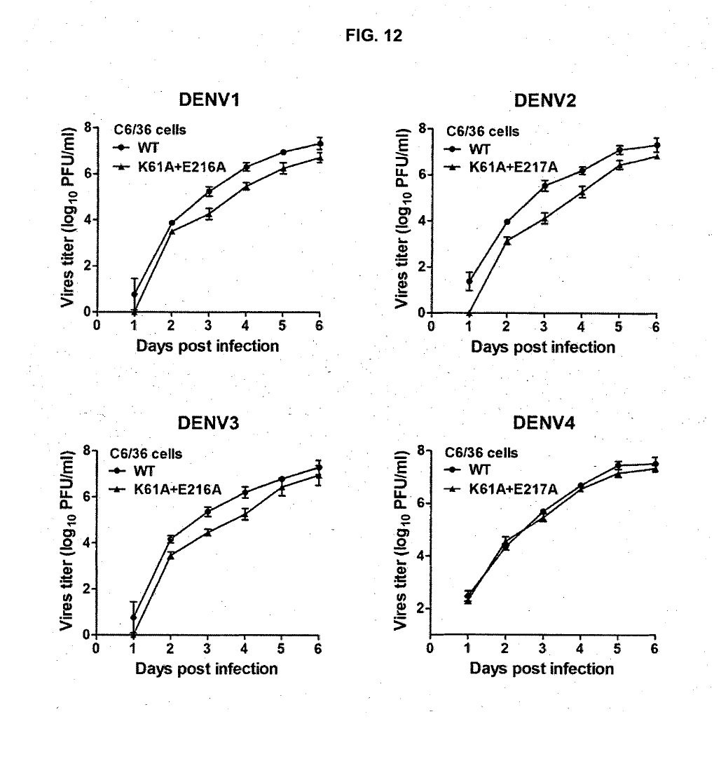

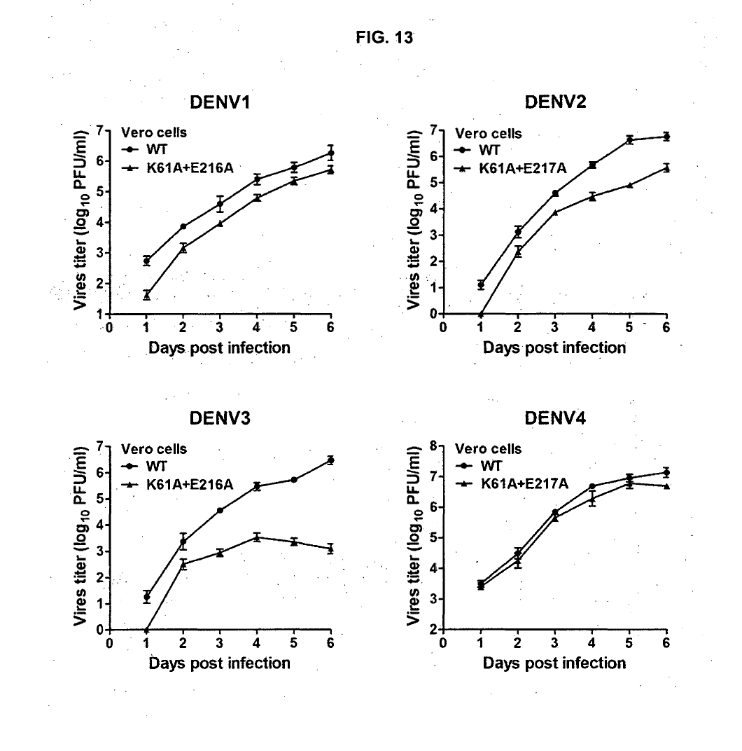

The growth curves, plaque morphology and a histogram showing IFN-β susceptibility of all DENV-1 to -4 viruses in comparison to each respective WT virus strain can be found in the FIGS. 12 to 15. As shown in FIGS. 12 and 13, both WT and mutant virus strains showed similar growth kinetics for all four DENV serotypes, except for DENV-3 where the double mutant growth was lower compared to the DENV-3 WT virus under the growth conditions used.

### Example 2

### The DENV 2′O-MTase Mutants are Highly Attenuated in Mice and Induce a Protective Immune Response

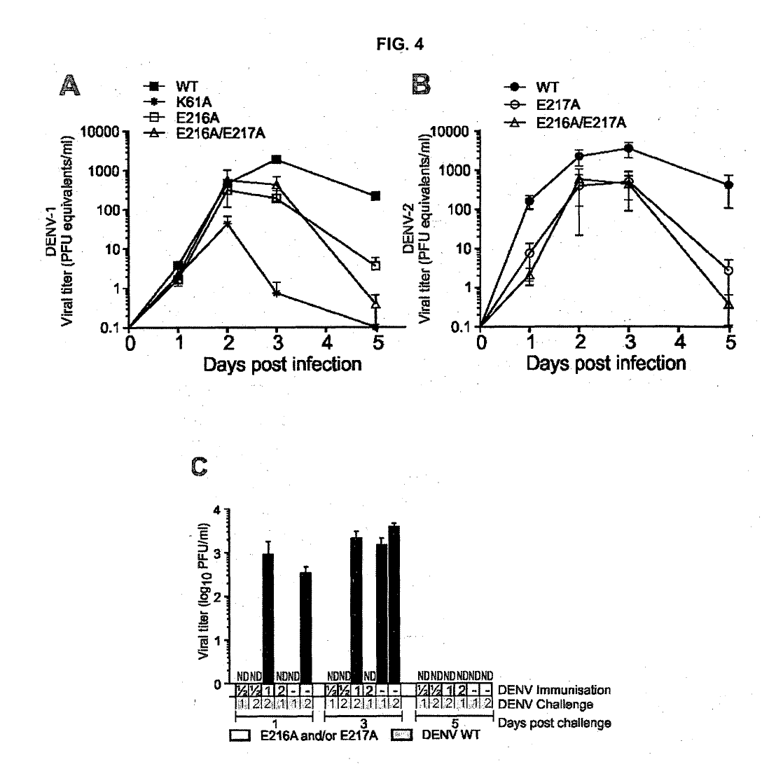

AG129 mice were infected with the WT and 2′-O-MTase mutants (called “E216A” for DENV-1 and “E217A” for DENV-2 from this point) to assess viral replication and immunogenicity in vivo. AG129 mice lack the receptors for type I and type II IFNs, and have been used widely for antiviral and vaccine testing. Mice were intraperitoneally (i.p.) infected with 2.75×10⁵ plaque-forming units (pfu) of WT or mutant viruses. The viremia result showed that mutating K61A or E216A in DENV-1 and mutating E217A in DENV-2 attenuated the virus compared to the WT virus (FIGS. 4(a) and (b)). Next, a combination of two MTase mutants (E216A and E217A) representing DENV-1 and DENV-2 were examined to address a potential competition effect that has been described for attenuated strains in humans and in mice. To this end, mice were injected i.p. with 2.75×10⁵ pfu of E216A or 2.75×10⁵ pfu of E217A or a combination of both (a total of 5.5×10⁵ pfu viruses). At 30 days post-vaccination, mice were challenged i.p. with 1′×10⁶ pfu of WT DENV-1 or 5×106 WT DENV-2. DENV-specific IgG titers and viremia were observed. All mice vaccinated with E216A and/or E217A were protected against homologous challenge (FIG. 4C), demonstrating that the immune response was protective even though the IgG titers in E216A and/or E217A-infected mice were 2 to 10 times lower than those in the WT virus-infected mice (FIGS. 4D and E).

A general concern for live attenuated vaccines is their theoretical potential to mutate back to WT under immune pressure. To address this in our system, virus from mice infected with mutant DENV1 or DENV2 was isolated at day 3 after infection and the mutations were found to be stable (FIG. 3c). To rule out that compensatory mutations were introduced into the viral genome, the input and output (day 3 after infection) virus was sequenced using Illumina® deep sequencing technology. As summarized in Table 2, only the single nucleotide polymorphisms (SNPs) responsible for the E216A or E217A mutation were found when comparing the sequences to wild-type DENV-1 or -2, respectively.

TABLE 2

| | | | | | | | |
|---|---|---|---|---|---|---|---|
| Virus | | Reference | Alternative | % | Variant | | p value |
| sample | Position | base | base | coverage | quality | depth | (log10) |

DENV-1 SNPs

| | | | | | | | |
|---|---|---|---|---|---|---|---|
| E216A in | 8220 | A | C | 99.82 | 189 | 5625 | −282 |
| E216A in | 8221 | A | C | | 198 | 5651 | −282 |
| E216A out 1 | 8220 | A | C | 99.57 | 47.1 | 27 | −45 |
| E216A out 1 | 8221 | A | C | | 36.3 | 27 | −42 |
| E216A out 2 | 8220 | A | C | 99.359 | 120 | 106 | −90 |
| E216A out 2 | 8221 | A | C | | 127 | 106 | −93 |
| E217A + | 8220 | A | C | 99.55 | 57.1 | 32 | −48 |
| E216A out 1 | | | | | | | |
| E217A + | 8221 | A | C | | 66 | 32 | −51 |
| E216A out 1 | | | | | | | |
| E217A + | 8220 | A | C | 99.57 | 36.1 | 74 | −48 |
| E216A out 2 | | | | | | | |
| E217A + | 8221 | A | C | | 45 | 75 | −54 |
| E216A out 2 | | | | | | | |

DENV-2 SNPs

| | | | | | | | |
|---|---|---|---|---|---|---|---|
| E217A in | 8219 | A | C | 99.77 | 199 | 5262 | −282 |
| E217A in | 8220 | G | C | | 505 | 5195 | −282 |
| E217A out 1 | 8219 | A | C | 99.7 | 25.1 | 76 | −28 |
| E217A out 1 | 8220 | G | C | | 60 | 74 | −51 |
| E217A out 2 | 8219 | A | C | 99.74 | 135 | 796 | −220 |
| E217A out 2 | 8220 | G | C | | 143 | 788 | −277 |
| E217A + | 8219 | A | C | 99.54 | 19 | 30 | −36 |
| E216A out 1 | | | | | | | |
| E217A + | 8220 | G | C | | 13.2 | 28 | −36 |
| E216A out 1 | | | | | | | |
| E217A + | 8219 | A | C | 99.62 | 31.1 | 61 | −45 |
| E216A out 2 | | | | | | | |
| E217A + | 8220 | G | C | | 35.1 | 60 | −48 |
| E216A out 2 | | | | | | | |

In: virus input;
out: virus output,
position: position in genome;
% coverage: % bases in the genome that were covered by at least one mapped read;
Variant Quality: The Phred-scaled average quality score for the variant position;
depth: number of reads mapped to the variant position;
p-value: the negative Phred-scaled probability of the variant being homozygous.

Similar experiments to ascertain the stability of the genetic mutation were performed in DENV-3 and -4. All the double mutant viruses were sequenced after five passages in Vero cells to confirm the retention of the E to A and K to A mutations in the active site of the 2′-O-methyltransferase and to identify additional mutations that might have been introduced during passaging. The inventors found that the attenuating mutations E to A and K to A were retained and that no additional mutations were introduced elsewhere into the virus genome during passaging, as can be seen, for example, in FIG. 3 and in the translation of the sequencing results from nucleic acid to amino acid sequence of SEQ ID NO: 9 to 12.

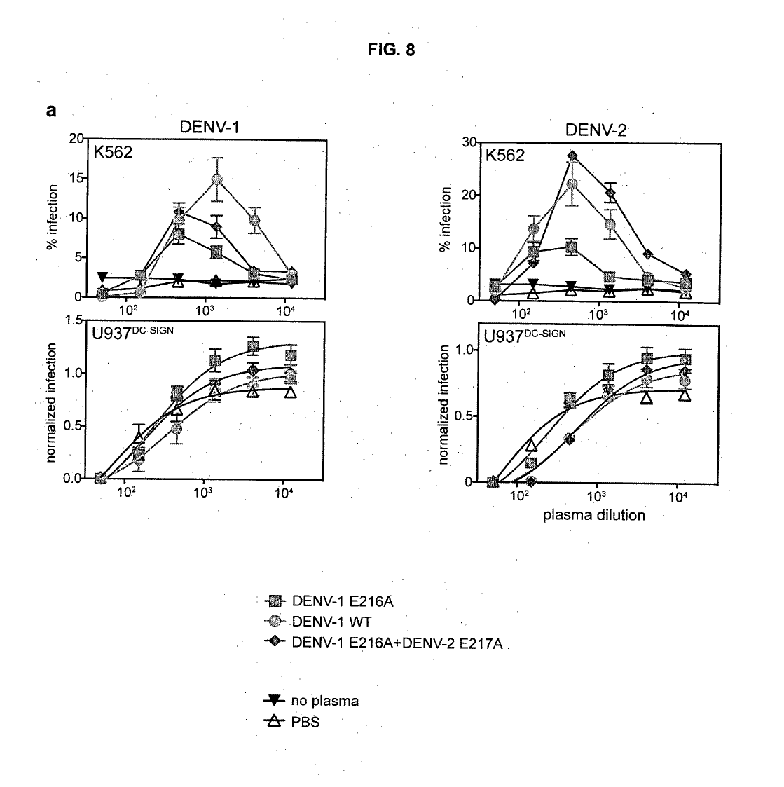

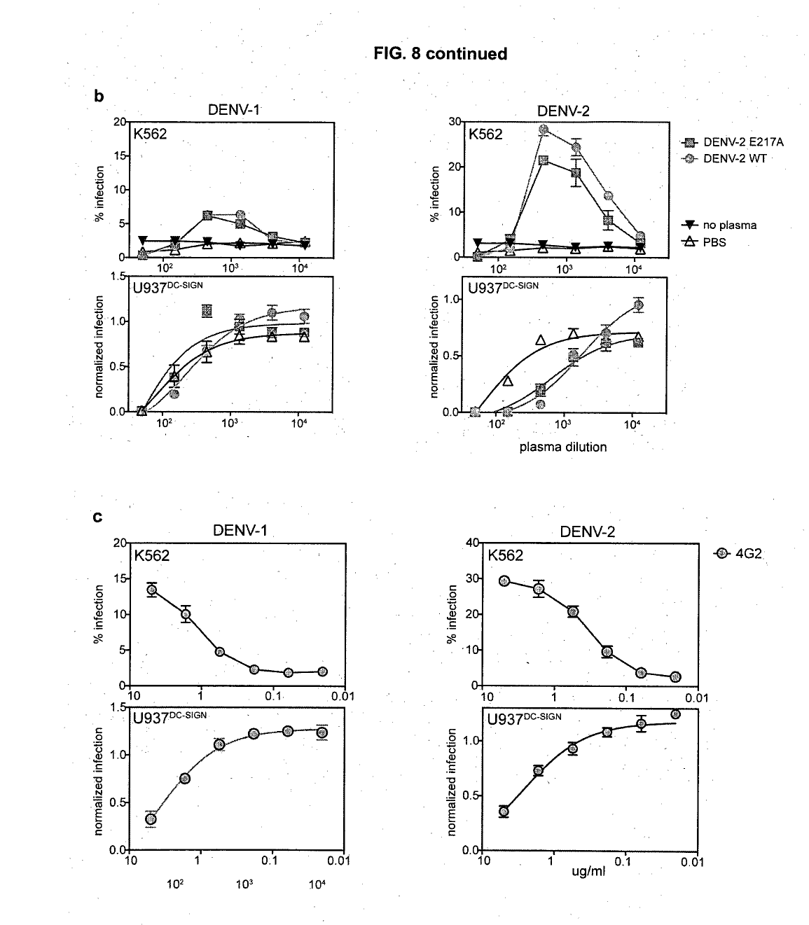

Next, the neutralization and infection-enhancing capacity of serum collected 30 days post-vaccination was compared (Table 3 and FIG. 8).

TABLE 3
Neutralization and antibody-dependent enhancement of infection (ADE) in
vaccinated AG219 mice.

| | Immunization: | | | | | | | |
|---|---|---|---|---|---|---|---|---|
| | NT50 (mean fold dilution ± SD) | | | | Max. ADE (mean fold dilution ± SD) | | | |
| | DENV-1 | p | DENV-2 | p | DENV-1 | p | DENV-2 | p |
| DENV-1 E216A | 252 ± 59 | | 388 ± 153 | # | 0.75 ± 0.27 | | 0.51 ± 0.16 | # |
| DENV-1 | 509 ± 307 | * | 556 ± 107 | | 0.98 ± 0.5 | * | 0.74 ± 0.2 | |
| DENV-2 E217A | 197 ± 188 | | 1035 ± 557 | * | 0.64 ± 0.22 | | 1.02 ± 0.22 | * |
| DENV-2 | 268 ± 118 | | 1548 ± 566 | * | 0.62 ± 0.14 | | 1.27 ± 0.29 | * |
| DENV-1 E216A + | 202 ± 78 | | 655 ± 261 | | 0.94 ± 017 | * | 1.05 ± 0.32 | * |
| DENV-2 E217A | | | | | | | | |
| PBS | 88 ± 66 | | 251 ± 228 | | 0.18 ± 0.01 | | 0.08 ± 0.01 | |

NT50 values are means ± SD of six to seven mice from two independent experiments. Max. ADE values are normalized against 4G2, which was used as an internal standard for infection efficiency per experiment. Values are means ± SD from six to seven mice from two independent experiments. Kruskal Wallis test with multiple comparisons:
*: p < 0.05 compared to PBS #: p < 0.05 compared to DENV-2

Mutant viruses cause the same or less antibody-dependent enhancement (ADE) than the respective wild-type viruses in the heterologous setting (0.51±0.16 vs. 0.74±0.2 for DENV-1 vaccination and ADE tested against DENV-2 and 0.64±0.22 vs. 0.62±0.14 for DENV-2 vaccination and ADE tested against DENV-1) (Table 3). More importantly, enhanced infection in vivo was not observed (FIG. 4C and FIG. 6B). These data suggest that vaccination with the E216A/E217A mutants does not cause ADE during heterologous challenge even though lower neutralizing Ab titers are generated by the mutant strains compared to the wild-type virus.

### Example 3

### Vaccinated Mice Generate a Non-Structural Protein-Specific CD8 T Cell Response

While antibodies are crucial to reduce the viral load by binding and neutralizing virus particles, T cells are necessary for efficient viral clearance. AG129 mice are not suitable to study T cell responses because of their lack of IFN-γ signaling, which is critical to activate T cells. Therefore, IFNAR mice lacking the receptor for IFN-α/β were used.

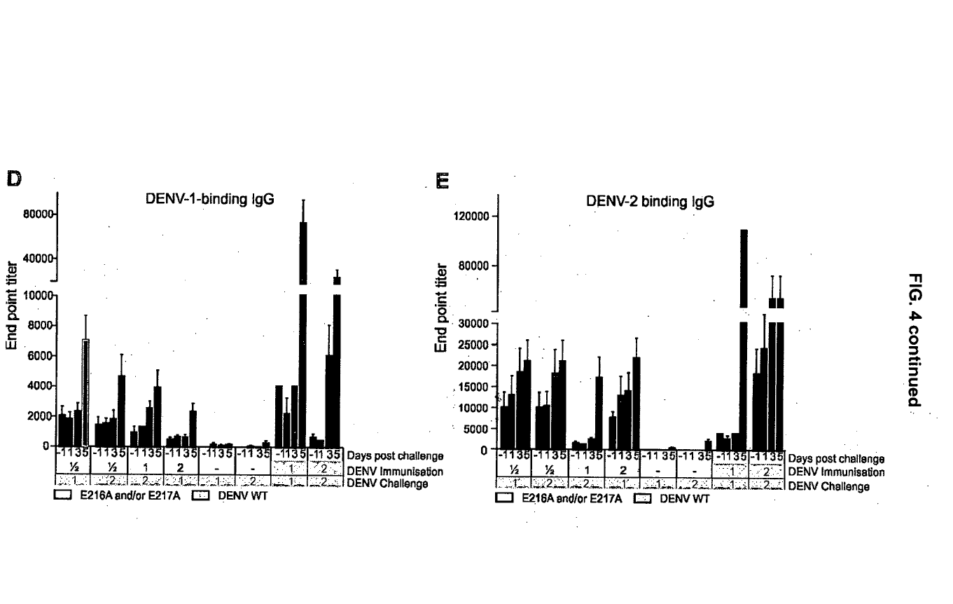

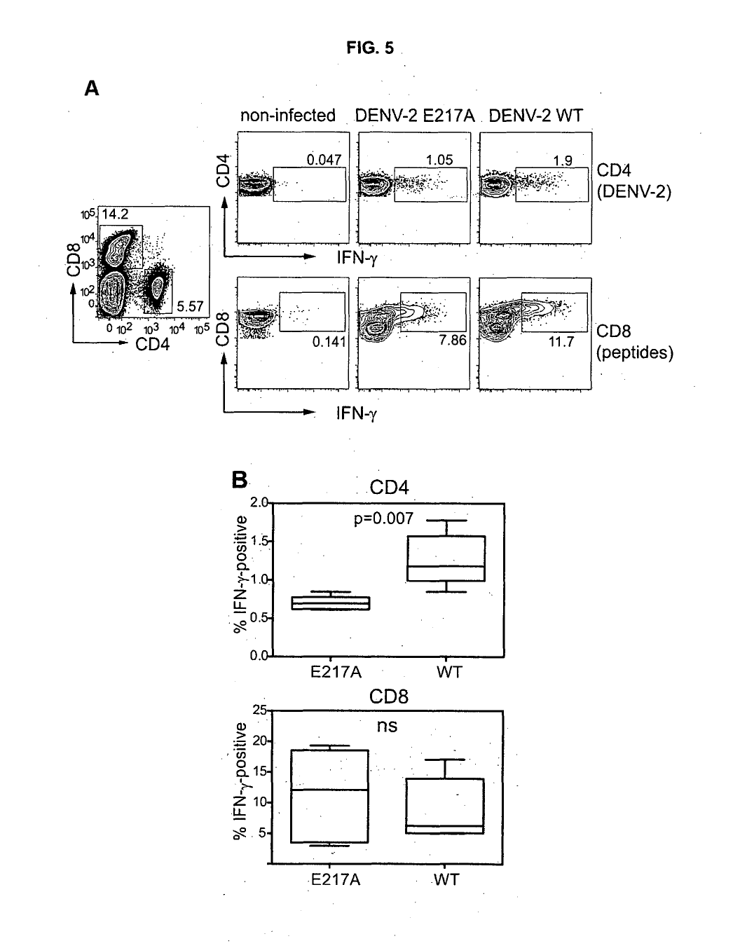

IFNAR mice were vaccinated with 2.75×10⁵ pfu DENV-2 E217A or DENV-2 WT, and spleens were harvested at day 7 for re-stimulation in vitro and detection of IFN-γ production (FIG. 5A). Mutant and WT virus elicited a strong CD4 and CD8 T cell response after re-stimulation with DENV-2. The CD4 response was weaker in E217A-vaccinated mice, likely due to the lower total viral load in E217A-vaccinated mice compared to mice vaccinated with the WT virus (FIG. 5B). To test for targeted DENV T cell response, splenocytes were re-stimulated with a pool of NS4B and NS5 CD8 peptides. No significant difference in the NS4B and NS5-specific T cell response was seen between mice vaccinated with E217A or WT DENV-2 (FIG. 5B). Taken together, DENV 2′-O-MTase mutants induce a T cell response and epitope presentation that is similar to WT infection.

### Example 4

### Vaccinated Mice are Protected Against Challenge with the Virulent DENV-2 Strain

DENV-1 strain 05K3126 and DENV-2 strain TSV01 do not cause pathology in mice. To test for protection against a more virulent strain, mice were vaccinated with DENV-1 E216A, DENV-2 E217A, a mixture of E216A and E217A, WT DENV-1 (Westpac) or WT DENV-2 (TSV01) or PBS, and challenged with the virulent DENV-2 strain D2Y98P 30 days later (FIG. 6). DENV-2 E217A protected against the homologous challenge (FIG. 6A). Vaccination with DENV-1 E216A protected 70% of the mice, showing limited cross-protection after infection with D2Y98P (FIGS. 6A and 6B). No enhanced disease was detected after heterologous challenge. Increased TNF-α levels were associated with pathology in the AG129 mouse model in the context of ADE. To further assess the possibility of ADE-associated pathology, TNF-α levels were measured in plasma three days after challenge. High levels of TNF-α were only detected in unvaccinated (PBS) mice, showing that TNF-α as a marker of pathology was independent of ADE, and that vaccination with E216A did not cause ADE after heterologous challenge. These data sets demonstrate that vaccination with E217A protects mice against challenge with an aggressive, virulent DENV-2 strain that causes 100% mortality in unvaccinated mice.

### Example 5

### DENV 2′-O MTase Mutants are Highly Attenuated in Macaques and Induce a Broad and Protective Immune Response

To assess the safety (viremia profile) and efficacy (neutralizing antibody response and protection against challenge) of the 2′-O-MTase mutant DENV vaccine approach in an immunologically competent host, three groups of Rhesus monkeys (RM) were vaccinated with different doses of E217A. One group received a low dose (1×10³ pfu), one group a medium dose (1×10⁴ pfu), and one group a high dose (1×10⁵ pfu) of E217A virus. Viremia was monitored during 10 days after vaccination. The E217A virus was severely attenuated, and no viremia was detected except for one animal (R0105) that had received a high dose (1×10⁵ pfu) and developed a low viremia (Table 4).

TABLE 4
Viremia in RMs vaccinated with different doses of DENV-2 E217A

| E217A | | | | | | | | | | | | | Mean | |
|---|---|---|---|---|---|---|---|---|---|---|---|---|---|---|
| dose | | | Viremia (log10 PFU/ml) at indicated day | | | | | | | | | | Peak | |
| (log10 | | | Post immunization | | | | | | | | | | titer | Duration |
| PFU) | Monkey | Gender | 1 | 2 | 3 | 4 | 5 | 6 | 7 | 8 | 9 | 10 | (SD) | days |
| 5.0 | R0319 | M | 0 | 0 | 0 | 0 | 0 | 0 | 0 | 0 | 0 | 0 | 0.4(0.8) | 0.8 |
| 5.0 | R0212 | F | 0 | 0 | 0 | 0 | 0 | 0 | 0 | 0 | 0 | 0 | | |
| 5.0 | R0105 | M | 0 | 0 | 1.5 | 1.6 | 0 | 0 | 1.6 | 0 | 0 | 0 | | |
| 5.0 | R0942 | F | 0 | 0 | 0 | 0 | 0 | 0 | 0 | 0 | 0 | 0 | | |
| 4.0 | R0055 | M | 0 | 0 | 0 | 0 | 0 | 0 | 0 | 0 | 0 | 0 | 0 | 0 |
| 4.0 | R0482 | F | 0 | 0 | 0 | 0 | 0 | 0 | 0 | 0 | 0 | 0 | | |
| 4.0 | R0098 | F | 0 | 0 | 0 | 0 | 0 | 0 | 0 | 0 | 0 | 0 | | |
| 3.0 | R0198 | F | 0 | 0 | 0 | 0 | 0 | 0 | 0 | 0 | 0 | 0 | 0 | 0 |
| 3.0 | R0195 | M | 0 | 0 | 0 | 0 | 0 | 0 | 0 | 0 | 0 | 0 | | |

Viruses were extracted for sequencing, and it was confirmed that the E217A mutation was retained in the virus extracted at days 3, 4 and 7 from this animal. Importantly, full virus genome sequencing of the viral RNA recovered at day 7 showed that no compensatory mutations were introduced (data not shown). All vaccinated monkeys developed neutralizing antibodies to DENV-2 on day 15 after vaccination (Table 5).

TABLE 5
Reciprocal neutralizing antibody titer in RMS vaccinated with DENV-2 E217A
*All animals were challenged with 1 × 10⁵ pfu of WT DENV-2 on day 64 post-vaccination.

| E217A | | | | | | | |
|---|---|---|---|---|---|---|---|
| dose | | | Reciprocal neutralizing antibody titer (PRNT50) | | | | |
| (log10 | | | Day post immunzation | | | Day post challenge* | |
| PFU) | Monkey | Gender | −1 | 15 | 30 | 15 | 30 |
| 5.0 | R0319 | M | <10 | 33 | 106 | 218 | 597 |
| 5.0 | R0212 | F | <10 | 122 | 90 | 400 | 378 |
| 5.0 | R0105 | M | <10 | 55 | 170 | 339 | 348 |
| 5.0 | R0942 | F | <10 | 87 | 122 | 187 | 301 |
| | GMT | | | 66 | 119 | 273 | 392 |
| 4.0 | R0055 | M | <10 | 46 | 447 | 411 | 386 |
| 4.0 | R0482 | F | <10 | 31 | 283 | 400 | 371 |
| 4.0 | R0098 | F | <10 | 29 | 80 | 190 | 405 |
| | GMT | | | 35 | 216 | 315 | 387 |
| 3.0 | R0198 | F | <10 | 56 | 77 | 344 | 534 |
| 3.0 | R0195 | M | <10 | 17 | 154 | 597 | 542 |
| 3.0 | R0200 | F | <10 | 15 | 66 | 406 | 640 |
| | GMT | | | 24 | 92 | 437 | 570 |

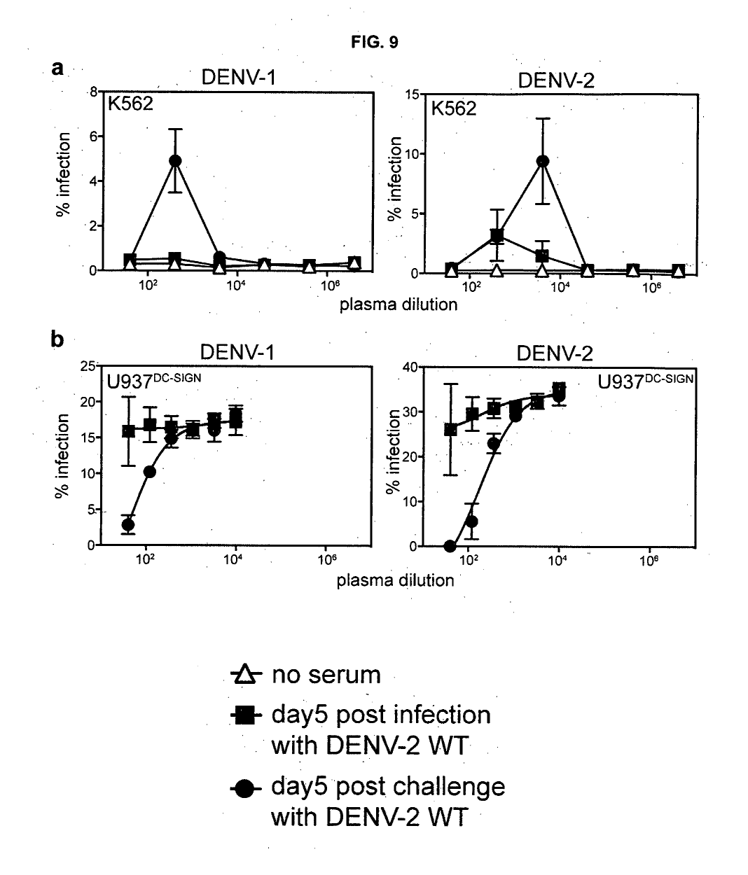

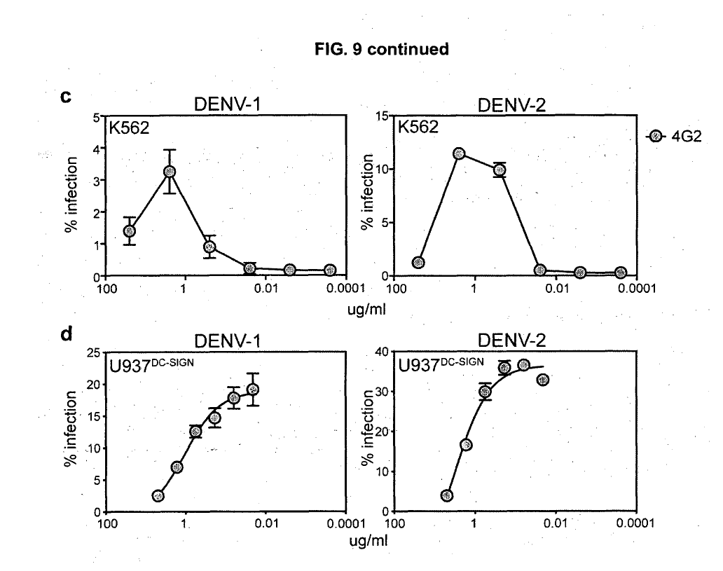

ADE was analyzed in a K562 assay and a similar enhancement pattern was observed for both heterologous and homologous infection in vitro: ADE correlated with the neutralizing titer, i.e. the higher the NT50, the higher the enhancement (FIG. 9). This argues against a physiologically relevant infection enhancement, which would only be expected after heterologous infection. By day 30 after vaccination, all monkeys including the ones with low dose vaccination developed high titers (GMT≧92) of neutralizing antibodies (Table 5). The monkeys were then challenged with 1×10⁵ pfu of WT DENV-2 on day 64 post-vaccination. No viremia was detected in any vaccinated monkey, whereas all four unvaccinated (PBS) controls had a mean peak virus titer of 2.5 (log 10) pfu/ml and mean viremia duration of 4.8 days (Table 6).

TABLE 6

| Viremia in E217A-vaccinated RMs after challenge with wild-type DENV-2* | | | | | | | | | | | | | |
|---|---|---|---|---|---|---|---|---|---|---|---|---|---|
| Group | | Dose | | | | | | | | | | Peak | Duration |
| (log10 | | (log10 | Viremia (log10 PFU/ml) by post challenge day | | | | | | | | | titer | days |
| PFU) | Monkey | PFU) | 1 | 2 | 3 | 4 | 5 | 6 | 7 | 8 | 9 | (SD) | (SD) |
| E217A | R0319 | 5 | 0 | 0 | 0 | 0 | 0 | 0 | 0 | 0 | 0 | | |
| 5.0 | R0212 | 5 | 0 | 0 | 0 | 0 | 0 | 0 | 0 | 0 | 0 | | |
| | R0105 | 5 | 0 | 0 | 0 | 0 | 0 | 0 | 0 | 0 | 0 | | |
| | R0942 | 5 | 0 | 0 | 0 | 0 | 0 | 0 | 0 | 0 | 0 | | |
| E217A | R0055 | 5 | 0 | 0 | 0 | 0 | 0 | 0 | 0 | 0 | 0 | | |
| 4.0 | R0482 | 5 | 0 | 0 | 0 | 0 | 0 | 0 | 0 | 0 | 0 | | |
| | R0098 | 5 | 0 | 0 | 0 | 0 | 0 | 0 | 0 | 0 | 0 | | |
| E217A | R0198 | 5 | 0 | 0 | 0 | 0 | 0 | 0 | 0 | 0 | 0 | | |
| 3.0 | R0195 | 5 | 0 | 0 | 0 | 0 | 0 | 0 | 0 | 0 | 0 | | |
| | R0200 | 5 | 0 | 0 | 0 | 0 | 0 | 0 | 0 | 0 | 0 | | |
| PBS | R0522 | 5 | 1.9 | 1.7 | 0 | 0 | 0 | 2.3 | 1.6 | 0 | 0 | 2.5(0.2) | 4.8(03) |
| | R0342 | 5 | 1.6 | 2.8 | 1.7 | 2.4 | 2.1 | 0 | 0 | 0 | 0 | | |
| | R1751 | 5 | 0 | 0 | 1.5 | 2.3 | 1.7 | 1.9 | 2.4 | 0 | 0 | | |
| | R0351 | 5 | 0 | 2.0 | 2.0 | 2.6 | 2.4 | 1.6 | 0 | 0 | 0 | | |

*Animals were challenged with 1 × 10⁵ pfu of WT DENV-2 on day 64 post-vaccination.

In all animals except one (R0055), anamnestic antibody responses were observed after challenge (Table 5). These data demonstrate that live, attenuated DENV MTase mutant virus, even when administrated at low dose (1×10³ pfu), can induce protective immunity in non-human primates.

The mechanism of attenuation of 2′-O-methyltransferase mutant viruses is their inability to evade the host cell's immune activation. One outcome of immune activation in infected cells is the production of interferon (IFN) to increase the production of antiviral proteins and pattern recognition receptor expression in infected and neighboring cells. Since mutant DENV strains are easily recognized by these antiviral proteins and pattern recognition receptors, double mutant viruses should be more susceptible to IFN-β pre-treatment of host cells compared to WT viruses. As expected, when the human monocytic cell line U937-DC-SIGN was infected with WT and mutant viruses, the mutant viruses were more susceptible to IFN-β pre-treatment (FIG. 15).

### Example 6

### IFN-β Pre-Treatment Inhibits 2′-O MTase Mutant Infection with the Involvement of IFIT1

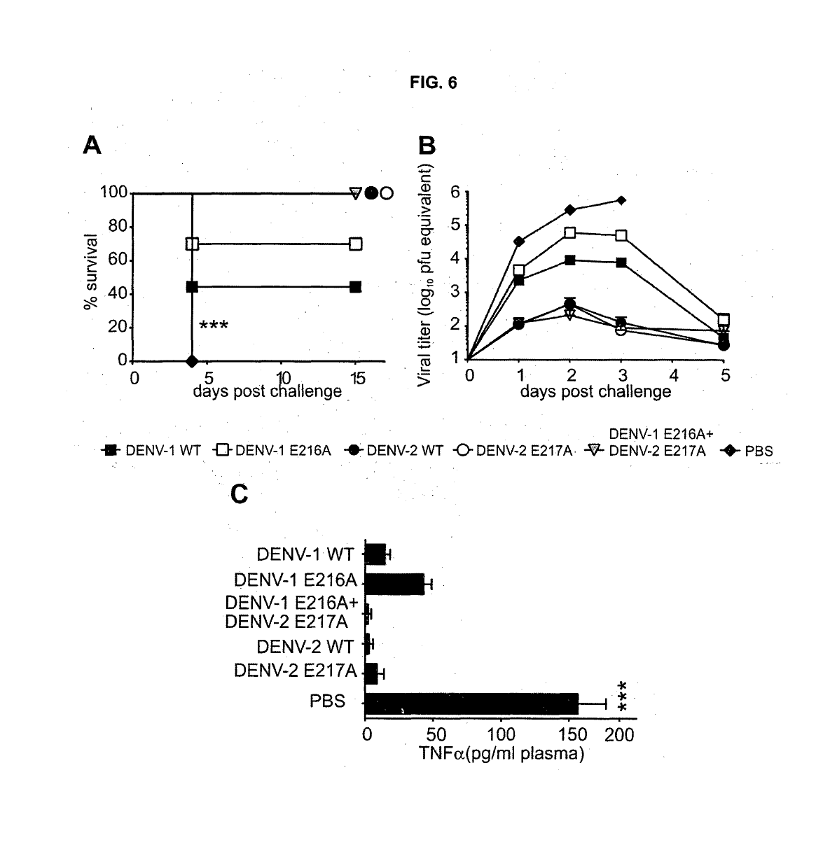

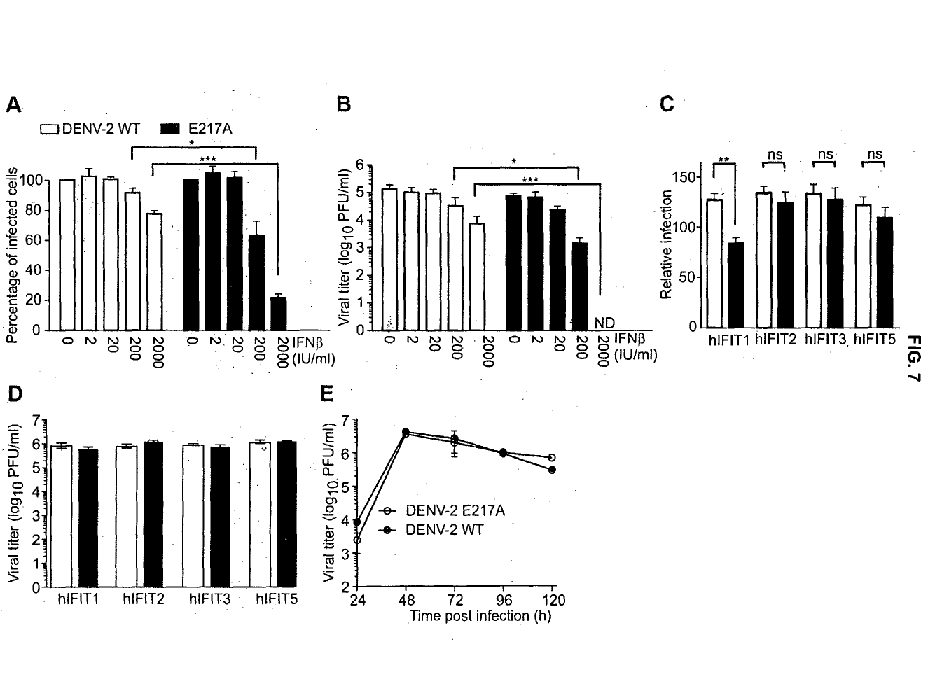

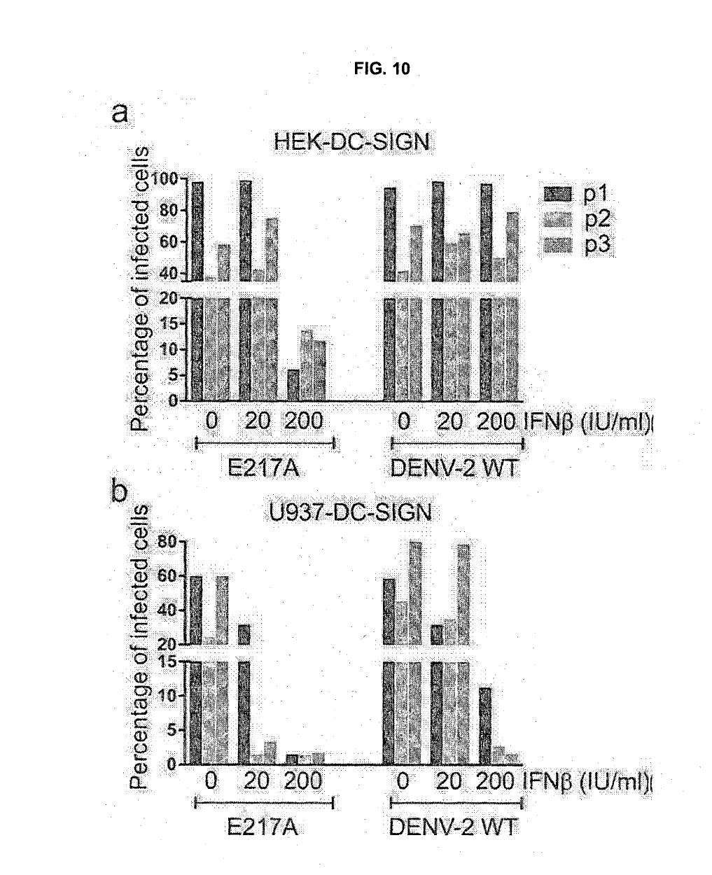

The 2′-O-methylation of the 5′ cap of WNV and coronavirus RNA functions to subvert innate host antiviral response through escape of IFIT-mediated suppression. To assess whether this is true for DENV as well, we pretreated HEK-DC-SIGN cells with an increasing dose of IFN-β for 24 h. While HEK-DC-SIGN cells are susceptible to type I IFN, they do not produce detectable levels of IFN-β after infection with mutant or WT DENV virus (data not shown). The IFN-β-treated cells were infected with WT or mutant E217A DENV-2. The E217A virus was significantly more sensitive to IFN-β pretreatment than the WT virus, as demonstrated by the percentage of infected cells (FIG. 7A), as well as the viral titers in culture supernatants (FIG. 7B). To test the stability of the mutation under IFN-β pressure and in different cell types, the virus was passaged in the presence of 0, 20 and 200 U/ml IFN-β in HEK-DC-SIGN and U937-DC-SIGN. As illustrated in FIG. 10, E217A virus was cleared in the presence of IFN-β, whereas wild-type virus resisted the IFN-β pressure in both cell lines. E217A isolated from passage three in HEK-DC-SIGN and from passage one in U937-DC-SIGN was isolated for sequencing.

The E217A mutation was retained and no compensatory mutations were introduced (data not shown). To elucidate the molecular mechanism of attenuation, human IFIT1, 2, 3, or 5 were over-expressed in HEK-DC-SIGN cells. The cells were infected with WT or mutant DENV-2 and assessed for the number of infected cells by flow cytometry (FIG. 7C). The WT virus infection was not affected, whereas E217A mutants were significantly inhibited by IFIT1, but not IFIT2, 3, or 5. However, IFIT1 over-expression did not completely block E217A infection nor did it affect virus output from the infected cells (FIG. 7D), suggesting that other IFN-mediated signals are involved in the response against DENV. Both mutant and WT virus show similar growth kinetics in untreated cells (FIG. 7E). It should be noted that the maximum antiviral effect of IFITs could be underestimated due to the low transfection efficiency (30-50%) of the IFIT expressing plasmids.

### Example 7

### Inability of 2′-O MTase Mutant Virus to Infect the Ae. aegypti Vector Decreases the Risk of Mutant Virus Transmission

The effect of 2′-O MTase mutation on viral fitness was compared in mosquito Ae. aegypti, the natural transmission vector for DENV. The mosquitoes were fed with blood containing DENV-2 WT or E217A. After the mosquitoes were fed at a titer of 1×10⁵ pfu/ml, significant differences in oral infection and dissemination between the WT and mutant viruses were observed 15 days post-infection (Table 7). The WT virus infected 29% of mosquitoes at the highest titer (1×10⁵ pfu/ml), but only 1-6% of mosquitoes at lower titers (1×10³ and 1×10⁴ pfu/ml). When orally fed with 1×10⁵ pfu/ml WT virus, approximately 10% of mosquitoes were infected; the WT virus disseminated in 24% of the mosquitoes (Table 7). When fed with 1×10³ and 1×10⁴ pfu/ml WT virus, the dissemination rates reached 1-4%. In contrast, the mutant virus was unable to infect the Ae. aegypti and, subsequently, no dissemination was observed for all titers (Table 7).

TABLE 7
Ae. aegypti susceptibility according to virus type and titer

| | Titer | Infected/total | | | | | Disseminated/total | | | | |
|---|---|---|---|---|---|---|---|---|---|---|---|
| | (log10 | female | | | | | female mosquitoes^{#} | | | | |
| Virus | PFU/ml) | mosquitoes (%)^{*} | | X² | dƒ | P-Value | (%) | | X² | dƒ | P-Value |
| WT | 5 | 24/82 | (29%) | 0.403 | 2 | 0.8175 | 20/82 | (24%) | 1.472 | 2 | 0.479 |
| | 4 | 1/72 | (1%) | 2.305 | 2 | 0.3159 | 1/72 | (1%) | 2.305 | 2 | 0.316 |
| | 3 | 3/53 | (6%) | 3.151 | 2 | 0.2069 | 2/53 | (4%) | 1.725 | 2 | 0.422 |
| E217A | 5 | 0/47 | (0%) | n/a | 2 | n/a | 0/47 | (0%) | n/a | 2 | n/a |
| | 4 | 0/40 | (0%) | n/a | 2 | n/a | 0/40 | (0%) | n/a | 2 | n/a |
| | 3 | 0/60 | (0%) | n/a | 2 | n/a | 0/60 | (0%) | n/a | 2 | n/a |

^{*}Infected: presence of virus in abdomen
^{#}Disseminated: presence of virus in thorax

To examine whether the E217A mutant could replicate in vivo, the WT and mutant viruses were intrathoracically inoculated into Ae. aegypti mosquitoes. Intrathoracic inoculation bypasses the mosquito mid-gut, which is the key barrier to establish infection during natural feeding route. Both WT and mutant viruses reached 100% infection rate upon intra-thoracic inoculation. The mean genome copy number reached 4.6×10⁹ and 6.2×10⁹, respectively (FIG. 11). The genome copy number of the WT virus was approximately 35% higher than that of the mutant virus (p=0.1054). Overall, the results demonstrate that the 2′-O-MTase mutant virus is compromised in vector fitness.

### Example 8

### Growth Kinetics of Double Mutant and Wildtype Virus Strains In Vitro

After electroporation of the reverse described RNA from double mutant and wildtype infectious clones into BHK21 cells, the released virus particles were further propagated on Vero cells for five passages to adapt the viruses to this cell line. The Vero cell line is recommended by the WHO for vaccine production and is suitable for the generation of master cell banks. After the fifth passage the viruses were used for further characterization. The growth kinetics of wildtype and double mutant viruses in C6/36 cells and Vero cells were analyzed. Briefly, cells were pre-seeded into 24-well plates (2×10⁵ cell/well) and then infected with WT and double mutant viruses at a multiplicity of infection (MOI) of 0.01. The secreted viruses in the supernatant were quantified by plaque assay at 1, 2, 3, 4, 5 and 6 days post-infection. As shown in FIGS. 12 and 13, both wildtype and mutant virus strains showed similar growth kinetics for all four DENV serotypes, except for DENV3, where the double mutant growth was lower compare to the wildtype virus at the growth conditions used (37° C. cell culture incubator).

### Example 9

### Genetic Stability of Double Mutant Viruses after Passaging In Vitro

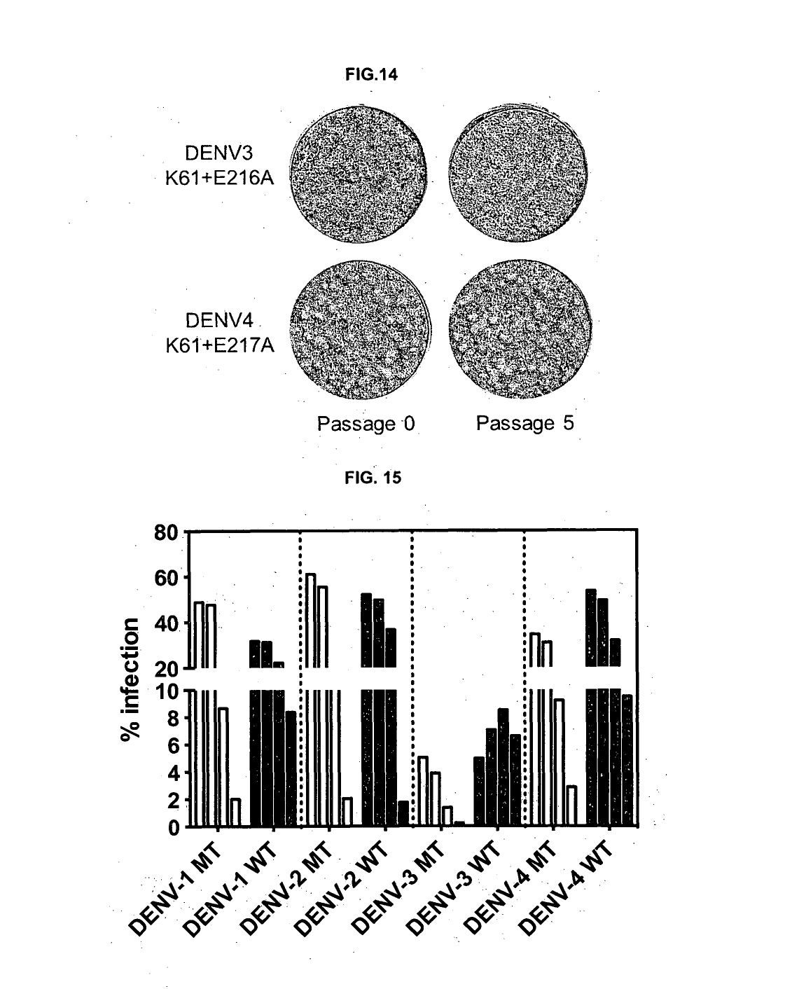

All the double mutant viruses were sequenced after five passages on Vero cells to confirm the retention of the E to A and K to A mutations in the active site of the 2′-O-methyltransferase and to identify additional mutations that might have been introduced during passaging. We found that the attenuating mutations E to A and K to A were retained and that no additional mutations were introduced elsewhere into the virus genome during passaging. As shown in FIG. 14, analysis of the plaque morphology demonstrated that the double mutant viruses recovered from viral RNA transfected cells (Passage 0), as well as viruses after culturing on Vero cells for 5 rounds (passage 5) had similar morphology.

### Example 10

### Increased Susceptibility of the Double Mutant Viruses to Interferon-Beta

The mechanism of attenuation of 2′-O-methyltransferase mutant, viruses is their inability to evade the host cell's immune activation. One outcome of immune activation in infected cells is the production of interferon-beta (IFN-β) to increase the production of antiviral proteins and pattern recognition receptors in infected and neighboring cells. Since mutant DENV strains are easily recognized by this antiviral proteins and pattern recognition receptors, double mutant viruses should be more susceptible to IFN-β pretreatment of host cells compared to wildtype viruses. As expected, when the human monocytic cell line U937-DC-SIGN was infected with wildtype and mutant viruses, the latter were more susceptible to IFN-β pretreatment (FIG. 15).

### Example 11

### Attenuation of Double Mutant DENV1, 2, 3 and 4 in Mice

Mice were infected with 10⁵ pfu wildtype or double mutant DENV-1, DENV-2 or DENV-4, or with 3,3×10⁴ pfu wildtype or double mutant DENV-3 and blood was collected at day 1, 3, 5 and 7 after infection for detection of viral RNA with qRT-PCR.

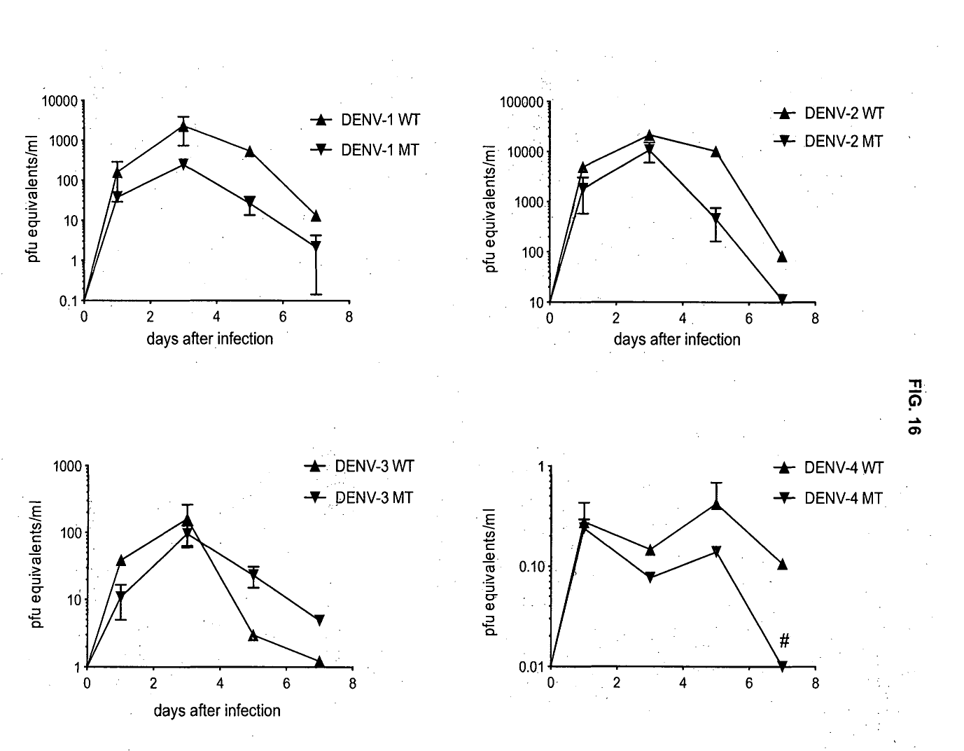

As observed in FIG. 16, the double mutant constructs for DENV-1 and DENV-2 were attenuated in AG129 mice. DENV-3 double mutant showed initial attenuation while the growth curve at later time points was similar to wildtype. The titers reached in mice were very low for both wildtype and double mutant DENV-3. Similarly, DENV-4 titers were very low or undetectable for both DENV-4 wildtype and double mutant strains.

### Example 12

### Antibody Response in Mice Vaccinated with Double Mutant DENV1, 2, 3 and 4 Viruses

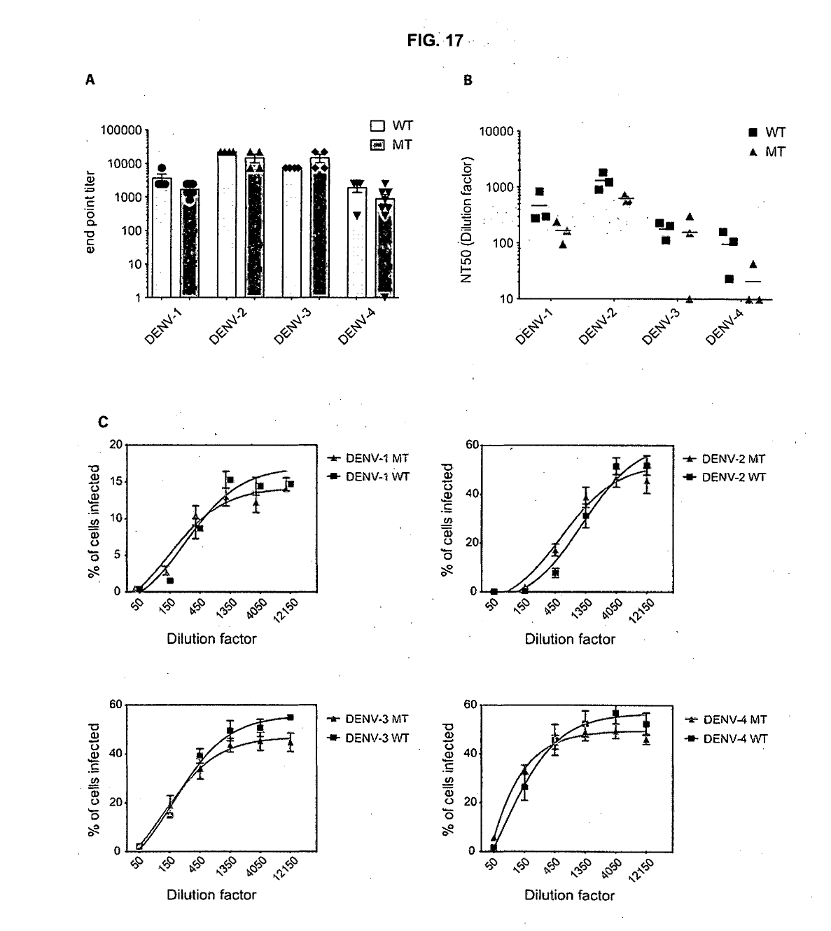

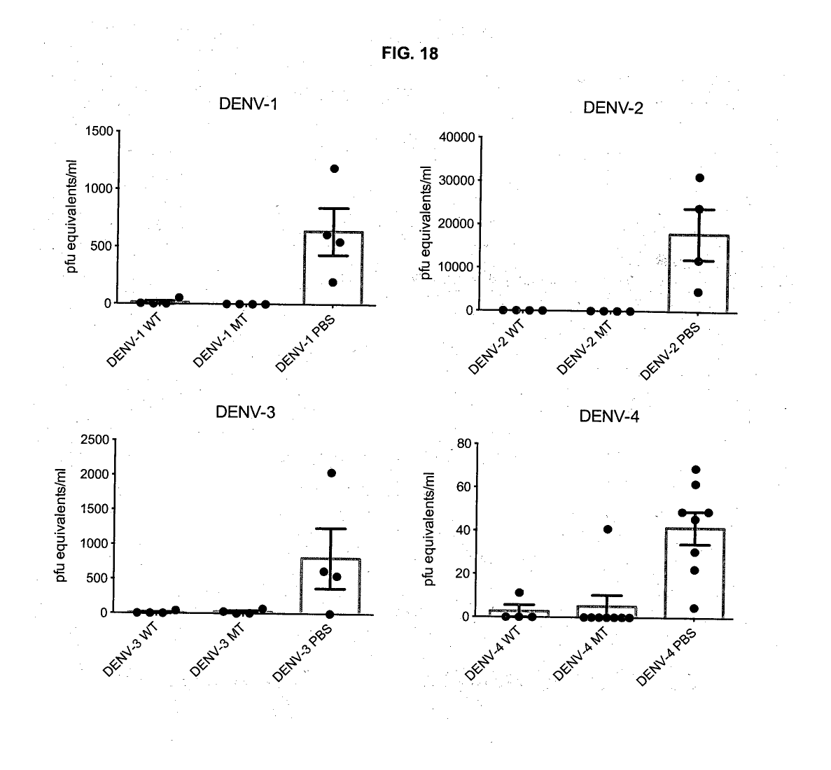

30 days after infection, DENV-specific antibodies in the plasma of infected mice were analyzed by ELISA and the Abs functional capacity to inhibit DENV infection was tested in a neutralization assay. Mice were infected with MT mutant dengue strains (grey bars) or with WT dengue strains (open bars) as shown in FIG. 17. ELISA plates were coated with UV-inactivated whole virus particles of DENV1, 2, 3 or 4 and plasma was added at decreasing concentrations to determine the end-point titer of DENV-specific antibodies. In all groups the ELISA antibody titers were comparable between mice infected with MT mutant dengue strains (grey bars) or with WT dengue strains (open bars) as shown in FIG. 17A Neutralizing titers were approximately 2-fold lower in DENV MT infected mice compared to mice infected with wildtype virus (FIGS. 17B and C), but the titers were still protective as shown in FIG. 18.

### Example 13

### Protection of Vaccinated Mice after Challenge with Wildtype Virus

Thirty days after vaccination with double mutant DENV-MT, DENV-WT or PBS, the mice were challenged with the homologous wildtype DENV virus (FIG. 18). Challenge dosages were as follows: WT DENV-1: 2×10⁷ pfu/mouse, WT DENV-2: 1×10⁷ pfu/mouse, WT DENV-3: 2×10⁷ pfu/mouse, WT DENV-4: 1.6×10⁸ pfu/mouse. At day 3 after challenge, the virus titer in the blood of the mice was assessed by qRT-PCR to test whether the mice were protected. All vaccinated mice except one mouse in the DENV-4 MT group were protected as shown by the absence of virus titers in the vaccinated mice compared to the unvaccinated mice (PBS). This one mouse had no detectable antibodies in both ELISA and neutralization assay (FIG. 18), which explains the lack of protection. DENV-2 D2Y98P infected mice in the PBS group all developed pathology and had to be eliminated, whereas mice in the WT and MT groups survived. In summary, these data show that all double mutant MT DENV strains induced protective immunity.

AG129 mice vaccinated once with a divalent mutant Dengue 1/Dengue 2 combination produced IgG titers of between 1:10,000 to 1:20,000, five days after challenge. No interference between the two serotypes of dengue MTase mutant vaccines could be observed in terms of viremia and antibody titers generated when two strains were given at the same time and in equal concentrations. Monkeys vaccinated with a single dose of Dengue 2 MTase mutant virus showed 100% seroconversion even when a dose as low as 1000 plaque forming units was administrated. Animals were fully protected against homologous challenge. These results clearly demonstrate the potential of 2′O-MTase Dengue mutants as safe, rationally designed live attenuated vaccine candidates. In the present invention, the inventors surprisingly showed that DENV bearing a mutation in the catalytic site of the 2′-O MTase replicate to high titers in cell culture and are highly attenuated in mice and rhesus monkeys. In some of the examples, it is shown that a mutation is stable over several passages and reversion to wild type has not been observed. To further improve safety, a second mutation in the catalytic tetrad can be introduced without affecting viability of the virus in vitro. A single dose administration to rhesus macaques (RM) leads to seroconversion and confers protection to homologous DENV challenge. Mice vaccinated with a single dose of a divalent (DENV1/2) formulation of the vaccine show comparable induction of antibodies as when vaccinated with a monovalent vaccine, demonstrating that there is no interference between the two serotypes of dengue MTase mutant vaccines. Taken together, these results clearly demonstrate that 2′-O MTase mutants harbor significant potential for future development of a tetravalent DENV vaccine. To our knowledge, this is the first live-attenuated rational vaccine under development, targeting optimal activation of the immune response while being severely attenuated.

Various dengue vaccine strategies are currently under development, including live attenuated virus, subunit vaccines, chimeric viruses, and DNA vaccines. The establishment of reverse genetic manipulation of DENV has greatly facilitated the generation of promising vaccine candidates. Reverse genetics is an approach, by which the function of a gene is analyzed by first modifying the gene, and subsequently studying the resulting phenotypical changes. The genetic modifications can be achieved by deleting, omitting or point-mutating sequences in the genetic code, resulting in gene silencing or aberrant gene function.

Reverse genetics is the opposite of the so-called forward genetics, whereby the mutant phenotype is first isolated, and then analyzed for its modified gene through standard molecular techniques. The recent progress in understanding the mechanism of attenuation of 2′-O MTase mutant flaviviruses has provided a novel approach for vaccine and antiviral development. Here, it is shown that MTase mutant E216A DENV-1 and E217A DENV-2 strains are stable in vitro, and safe and immunogenic in vivo. Importantly, enhancement of infection was not observed after heterologous infection of vaccinated mice. A commonly used approach to address ADE in vitro is to infect K562 cells in the presence of antibodies. Virus alone is not able to infect K562 cells efficiently, whereas virus-antibody immune complexes bind to K562 cells via Fc-γ receptors (FcγR), assisting the internalization of the virus and infection of the cells. It was found that K562 cells could be infected in the presence of serum from vaccinated mice and monkeys at dilutions that were approximately 50% neutralizing in the U937-DC20 SIGN system (FIGS. 8 and 9). This is in line with a previous report, which found that even strongly neutralizing antibodies are enhancing at concentrations that are close to the 50% neutralizing titer.

Live attenuated dengue vaccine candidates have several advantages. Importantly, they can induce long lasting humoral and cellular immune responses to both structural and non-structural viral proteins. In this study, it was shown that a CD8 response to NS4B and NS5 peptides is similar in mice vaccinated with mutant or WT virus, suggesting that the response is qualitatively equivalent. Chimeric viruses, using the same backbone for all four DENV serotype glycoproteins, would induce a type-specific response restricted to the structural proteins of one DENV serotype.

The interdependence of the T and B cell response for the efficient generation of immune memory has been demonstrated in a number of human studies. It is possible that an attenuated, non-chimeric DENV, including all naturally occurring T and B cell epitopes, would be able to confer long-term immunity to reinfection after only one vaccination, as seen for natural DENV infections. A single-dose vaccine would facilitate the logistics of a vaccination program and would significantly reduce its cost compared to candidates requiring several booster vaccinations. The 2′-O MTase mutant DENV vaccine approach, with a known mechanism of attenuation, can be readily generated using a reverse genetics system. This is in contrast to the method to develop live, attenuated vaccines by passaging of WT viruses in cell lines, leading to the introduction of random mutations.

The reverse genetics system-based rational vaccine ensures that the vaccine maintains the attenuated genotype. Additionally, a tetravalent formulation would contain the same attenuating mutation in all four serotype recombinant vaccine strains, making the generation of a more pathogenic virus by intra-vaccine strain recombination impossible. Moreover, recombination in cell culture is hardly observed in flaviviruses, suggesting that flaviviruses are not prone to evolution by recombination. By introducing additional mutations in the K-D-K-E tetrad of 2′-O MTase, further safety and attenuation can be achieved.

The present invention thus demonstrates that the 2′-O MTase E217A virus is attenuated in mice and monkeys. Studies in human HEK293 cells show increased susceptibility of DENV2 E217A mutant to IFN-β in vitro, suggesting that DENV E217A mutants will be attenuated in humans as well. In the monkey vaccination experiments, one monkey out of four in the high dose group experienced peak viremia of about 100 pfu, which is comparable to other live attenuated vaccine candidates. Indeed, replication of the attenuated vaccine is desirable in order to induce a strong protective cellular immune response.

Replication should be restricted enough to preclude onset of illness, whereas sub-clinical symptoms such as mild rash, transient leukopenia, and mildly elevated liver enzyme values are generally accepted. Furthermore, studies with murine hepatitis virus have shown that MTase mutants are highly attenuated in its natural host, induce IFN, which could further induce the immunogenicity of a vaccine, and are genetically stable in vivo. Moreover, the replication level of WNV 2′-O MTase mutant in mice was largely decreased in the spleen, serum, or brain in comparison with the WT WNV infection. Intracranial inoculation of 1×10⁵ pfu of 2′-O-MTase mutant WNV did not cause any mortality and morbidity in mice, demonstrating the safety of this vaccine approach. Taken together, these evidences demonstrate the safety and immunogenicity of the MTase-mutant vaccine approach.

Material and Methods

Cells

BHK-21, C6/36, and HEK-293 were purchased from the American type culture collection (http://www.atcc.org). HEK-293 cells expressing DC-SIGN were obtained by lentiviral transfection and subsequent cell sorting. All cells were maintained in minimal essential medium supplemented with fetal bovine serum (5%-10%):

Recombinant MTase Preparation and Methylation Assays.

WT MTases representing the N-terminal 262 and 296 amino acids of DENV-1 and -2 NS5, respectively, were cloned, expressed, and purified. Mutagenesis of MTase was performed using a standard protocol of overlap PCR. The complete sequence of each mutant MTase was verified by DNA sequencing. N7 and 2′-O methylation assays were performed as described using methods known to the skilled person in the art.

Construction of Attenuated Viruses DENV-1, 2, 3 and 4 with Two Mutations

To reduce the risk of genetic reversion in the mutated viruses we further modified the virus genome and introduced an additional mutation in the KDKE domain in addition to the E to A mutation described initially. The same mutation strategy was applied for all four serotypes and the position of the mutations are summarized in Table 1. These viruses are called double mutants. Full-length infectious cDNA clones of DENV-1 (Western Pacific 74 strain), DENV-2 (TSV01 strain), DENV-3 (D3MY05-34640) and DENV-4 (D4MY01-22713) were used to generate WT and mutant viruses. In short, the two mutations were engineered into MTase domain using the QuikChange® II XL Site-Directed Mutagenesis Kit (Stratagene) according to the instructions. Subsequently, the genome-length RNAs of DENV-1 to DENV-4 were in vitro transcribed from corresponding cDNA plasmids that were pre-linearized using a T7 mMESSAGE mMACHINE kit (Ambion). Finally, the RNAs were electroporated into BHK21 cells and cultured in 5% CO₂ in a 30° C. incubator.

Preparation and Characterization of Recombinant DENV.

Full-length infectious cDNA clones of DENV-1 (Western Pacific 74 strain) and DENV-2 (TSV01 strain) were used to generate WT and mutant viruses. A standard mutagenesis protocol was used to engineer mutations into the MTase region. The protocols for in vitro transcription, RNA transfection, IFA, plaque assay, and growth kinetics are known to the skilled addressee.

Growth Kinetics of Double Mutant and Wildtype Virus Strains In Vitro

After electroporation of the reverse described RNA from double mutant and wildtype infectious clones into BHK21 cells, the released virus particles were further propagated on Vero cells for five passages to adapt the viruses to this cell line. The Vero cell line is recommended by the WHO for vaccine production and is suitable for the generation of master cell banks. After the fifth passage the viruses were used for further characterization. The growth kinetics of wildtype and double mutant viruses in C6/36 cells and Vero cells were analyzed. Briefly, cells were pre-seeded into 24-well plates (2×10⁵ cell/well) and then infected with WT and double mutant viruses at a multiplicity of infection (MOI) of 0.01. The secreted viruses in the supernatant were quantified by plaque assay at 1, 2, 3, 4, 5 and 6 days post-infection.

Genetic Stability of Double Mutant Viruses after Passaging In Vitro

All the double mutant viruses were sequenced after five passages on Vero cells to confirm the retention of the E to A and K to A mutations in the active site of the 2′-0-methyltransferase and to identify additional mutations that might have been introduced during passaging.

Mice

Female or male 6-8 week old IFN alpha/beta/gamma receptor deficient mice (AG129) were purchased from B&K Universal Limited with permission from Dr. M. Aguet (ISREC, School of Life Sciences Ecole Polytechnique Fédérale (EPFL)). All mice were bred and kept under specific pathogen-free conditions in the Biomedical Resource Centre, Singapore. For vaccination comparison between WT and E271A strains, BHK-21 derived viruses were used. Only for challenge experiments, was DENV produced in C6/36 cells used.

Attenuation of Double Mutant DENV1, 2, 3 and 4 in Mice

Mice were infected with 10⁵ pfu wildtype of double mutant DENV-1, DENV-2 or DENV-4, or with 3,3×10⁴ pfu wildtype or double mutant DENV-3 and blood was collected at day 1, 3, 5 and 7 after infection for detection of viral RNA with qRT-PCR.

Antibody Response in Mice Vaccinated with Double Mutant DENV1, 2, 3 and 4

Thirty days after infection, DENV-specific antibodies in the plasma of infected mice were analyzed by ELISA and the Abs functional capacity to inhibit DENV infection was tested in a neutralization assay. Mice were infected with MT mutant dengue strains or with WT dengue strains. ELISA plates were coated with UV-inactivated whole virus particles of DENV1, 2, 3 or 4 and plasma was added at decreasing concentrations to determine the end-point titer of DENV-specific antibodies.

Protection of Vaccinated Mice after Challenge with Wildtype Virus

Thirty days after vaccination with double mutant DENV-MT, DENV-WT or PBS, the mice were challenged with wildtype DENV virus, using different strains than the ones used for vaccination (FIG. 18). Challenge dosages were as follows: WT DENV-1: 2×10⁷ pfu/mouse, WT DENV-2: 1×10⁷ pfu/mouse, WT DENV-3: 2×10⁷ pfu/mouse, WT DENV-4: 1.6×10⁸ pfu/mouse. The challenge strains used were DENV-1 05K3126, DENV-2 D2Y98P, DENV-3 VN32/96 (Genbank EU482459) and DENV-4 TVP-360 (GU289913.1). At day 3 after challenge, the virus titer in the blood of the mice was assessed by qRT-PCR to test whether the mice were protected.

Rhesus Monkey Study

All the animal experimental procedures were approved by and carried out in strict accordance with the guidelines of the Animal Experiment Committee of State Key Laboratory of Pathogen and Biosecurity, Beijing, China. Fourteen RMs, weighing from 3.4 to 5.0 kg, were prescreened negative for antibodies against dengue and Japanese encephalitis virus by IFA.

Animals were randomly divided into four groups and vaccinated s.c. in the deltoid region of left arm with 0.5 ml of DENV2-E217A containing 10⁵ pfu, 10⁴ pfu, 10³ pfu, or PBS. Blood was collected from each RM daily post-vaccination for 10 days to detect viremia. For neutralizing antibody tests, bloods were taken immediately before vaccination (day −1) and then on days 15, and 30 post-vaccination. On day 64 post-vaccination, all monkeys were challenged by s.c. inoculation with 0.5 ml containing 5×10¹⁰ pfu of DENV-2 (TSV-01). For the following 9 days, blood was collected for determination of viremia. Neutralizing antibody levels in serum were measured by plaque reduction neutralization test on days 15 and 30 post-challenge.

Determination of Viremia in Monkey Sera.

The concentration of DENV2 TSV01 in serum samples was determined by plaque assay in BHK cell monolayers in 12-well plates. Undiluted serum or serial 10-fold dilutions of serum were inoculated onto BHK cells. After 1 h of adsorption at 37° C., wells were overlaid with 1 ml of DMEM supplemented with 2% FBS and 1% agarose. Plates were incubated for 4 days at 37° C. in 5% CO2. Monolayers were fixed by addition of 1 ml of 4% formalin solution to the overlay medium. After 1 h of fixation at room temperature, the fixative was removed, wells were washed with water, and monolayers were stained with 1% crystal violet in 70% methanol. Plaques were counted, and titers were expressed as pfu per milliliter.

Plaque Reduction Neutralization Test

For determination of dengue virus-neutralizing antibody titers, serial two-fold dilutions of serum (starting at a serum dilution of 1:10) were mixed with equal volumes of a suspension of ˜500 pfu of DENV-2-TSV01/ml. The serum-virus mixtures were incubated at 37° C. for 1 h and tested (0.2 ml/well) for concentration of infectious virus using the plaque assay described above.

The neutralization titer was defined as the lowest serum dilution at which the infectious virus concentration was reduced by 50% from the concentration found when virus was incubated with culture medium.

Interferon Pretreatment

Cells were seeded at 1×10⁵ per well in a 24-well plate and treated 24 hours prior to infection with medium or varying concentrations of human recombinant IFN-beta (Immunotools). Cells were then infected at an MOI of 1 with wildtype or MTase mutant virus (TSV01), respectively, incubated for 72 hours and harvested and processed for flow cytometry as described. Supernatants were collected for plaque assay.

Detection of Infection by Flow Cytometry

To determine the percentage of infected cells, cells were harvested, washed in PBS and fixed and permeabilized with Cytofix/Cytoperm. Intracellular dengue E protein was stained with antibody 4G2 conjugated to Alexa 647 and fluorescent cells were measured by flow cytometry. IgG ELISA 96-well polystyrene plates were coated with concentrated, heat inactivated dengue virus.

Plates were incubated overnight at 4° C. Before use, plates were washed three times in PBS (pH 7.2) containing 0.05% Tween-20 (PBS-T). Non-specific binding was blocked with 2% non-fat dry milk diluted in PBS (PBS-M) for 2 h at room temperature (RT). After washing, sera were diluted 1:50 in PBS-M, heat inactivated for 1 hour at 55° C. and three-fold serial dilutions were added to the wells. Plates were incubated for 1 h at RT, followed by three washes with PBS-T.

Peroxidase-conjugated rabbit anti-mouse IgG, in PBS-M was added, followed by 1 h of incubation at RT and three additional washes with PBS-T. TMB was used as the enzyme substrate. The reaction was stopped with 1 M HCl and the optical densities were read at 450 nm using an automatic ELISA plate reader. Endpoint titers were defined as the lowest dilution of plasma in which binding was twofold greater than the mean binding observed with the negative controls.

Statistical Analysis

Statistical tests were performed with GraphPad Prism software, using students t test or two-way ANOVA as indicated in the figure legends.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, concentration etc). A range of values will be understood to include all values within the range, including the values defining the range, and values adjacent to the range, which lead to the same or substantially the same outcome as the values immediately adjacent to that value which defines the boundary to the range.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

1. A method of eliciting an immune response comprising administration of a mutated flavivirus comprising at least two mutations in a nucleic acid sequence encoding for a non-structural protein 5 of the flavivirus sequence, wherein the at least two mutations lead to inactivation of 2′O-methyltransferase activity of the non-structural protein 5.

2. The method of claim 1, wherein the at least two mutations are in the KDKE motif.

3. The method of claim 1, whereby the mutations result in replacement of a polar amino acid in the KDKE motif of the non-structural protein 5 of the flavivirus.

4. The method of claim 1, wherein the mutated flavivirus comprises at least one further mutation in a motif selected from the group consisting of a GTP-pocket, a SAM-pocket and a RNA binding site of the non-structural protein 5 of the flavivirus.

5. The method of claim 4, wherein the further mutation results in replacement of a polar amino acid in the GTP-pocket, and/or SAM-pocket and/or RNA binding site of the non-structural protein 5 of the flavivirus.

6. The method of claim 1, wherein the at least one mutation results in the replacement of a polar amino acid with a non-polar amino acid at Lysine 61, or Lysine 181, or glutamic acid 217 or equivalent respective amino acid positions in the KDKE motif of the non-structural protein 5 of the flavivirus.

7. The method of claim 6, wherein the at least one mutation results in the replacement of a polar amino acid with a non-polar amino acid at Lysine 61 or Glutamic acid 217 or equivalent respective amino acid positions of the non-structural protein 5 of the flavivirus.

8. The method of claim 1, wherein the mutations that result in the replacement of a polar amino acid with a non-polar amino acid is the amino acid at Lysine 61 and Glutamic acid 217 or at equivalent respective positions in the KDKE motif of the non-structural protein 5 of the flavivirus.

9. The method of claim 4, wherein the further mutation is in the GTP-pocket at Lysine 14 and/or Lysine 29 or at equivalent respective amino acid positions in the GTP-pocket of the non-structural protein 5 of the flavivirus.

10. The method of claim 4, wherein the further mutation is in the SAM-pocket at Isoleucine 147 or at equivalent respective amino acid positions in the SAM-pocket of the non-structural protein 5 of the flavivirus.

11. The method of claim 4, wherein the further mutation is in the RNA binding site at Glutamic acid 35 and/or Tryptophan 87 or at equivalent respective amino acid positions in the RNA-binding site of the non-structural protein 5 of the flavivirus.

12. The method of claim 1, wherein at least two amino acids are replaced with non-polar amino acid at positions selected from the group consisting of Lysine 61, Lysine 181, Glutamic acid 216, and equivalent respective amino acids positions in the KDKE motif.

13. The method of claim 12, wherein the mutated flavivirus comprises further mutations comprise mutations at positions selected from the group consisting of Lysine 14 and Lysine 29 in the GTP-pocket, Isoleucine 147 in the SAM-pocket, Glutamic acid 35 and Tryptophan 87 in the RNA binding site and equivalent respective amino acids positions.

14. The method of claim 1, wherein the mutated flavivirus has three mutations in the nucleic acid sequence encoding for a non-structural protein 5 of the flavivirus sequence, whereby the three mutations result in inactivation of 2′O-methyltransferase activity of the non-structural protein 5.

15. The method of claim 1, wherein the mutated flavivirus is a mutated attenuated virus.

16. The method of claim 1, wherein the mutated flavivirus is a mutated dengue virus.

17. The method of claim 16, wherein the mutated dengue virus comprises at least one or at least two dengue virus ribonucleic acid sequences selected from the group consisting of dengue virus 1 ribonucleic acid sequence (DENV-1), dengue virus 2 ribonucleic acid sequence (DENV-2), dengue virus 3 ribonucleic acid sequence (DENV-3) and dengue virus 4 ribonucleic acid sequence (DENV-4).

18. The method of claim 6, wherein the non-polar amino acid is an Alanine.

19. The method of claim 1, wherein the mutated flavivirus is a mutated tick borne encephalitis virus (TBEV) of any serotype.

20. A method of vaccination, comprising administration of at least one vaccine, which is a mutated flavivirus comprising at least two mutations in a nucleic acid sequence encoding for a non-structural protein 5 of the flavivirus sequence, wherein the at least two mutations lead to the inactivation of 2′O-methyltransferase activity of the non-structural protein 5.

21. (canceled)

22. The method of claim 1, wherein the mutated flavivirus is a mutated DENV-1 dengue virus, wherein Glutamic Acid 216 in the KDKE motif of the non-structural protein 5 of the DENV-1 dengue virus is replaced by Alanine.

23. The method of claim 1, wherein the mutated flavivirus is a mutated DENV-1 dengue virus, wherein Lysine 61 and Glutamic Acid 216 in the KDKE motif of the non-structural protein 5 of the DENV-1 dengue virus are replaced by Alanine.

24. The method of claim 1, wherein the mutated flavivirus is a mutated DENV-2 dengue virus, wherein Glutamic Acid 217 in the KDKE motif of the non-structural protein 5 of the DENV-2 dengue virus is replaced by Alanine.

25. The method of claim 1, wherein the mutated flavivirus is a mutated DENV-2 dengue virus, wherein Lysine 61 and Glutamic Acid 217 in the KDKE motif of the non-structural protein 5 of the DENV-2 dengue virus are replaced by Alanine.

26. The method of claim 1, wherein the mutated flavivirus is a mutated DENV-3 dengue virus, wherein Glutamic Acid 216 in the KDKE motif of the non-structural protein 5 of the DENV-3 dengue virus is replaced by Alanine.

27. The method of claim 1, wherein the mutated flavivirus is a mutated DENV-3 dengue virus, wherein Lysine 61 and Glutamic Acid 216 in the KDKE motif of the non-structural protein 5 of the DENV-3 dengue virus are replaced by Alanine.

28. The method of claim 1, wherein the mutated flavivirus is a mutated DENV-4 dengue virus, wherein Glutamic Acid 217 in the KDKE motif of the non-structural protein 5 of the DENV-4 dengue virus is replaced by Alanine.

29. The method of claim 1, wherein the mutated flavivirus is a mutated DENV-4 dengue virus, wherein Lysine 61 and Glutamic Acid 217 in the KDKE motif of the non-structural protein 5 of the DENV-4 dengue virus are replaced by Alanine.

30. The method of claim 20, wherein an immunization is obtained by at least one time administration of the mutated flavivirus.

31. The method of claim 20, wherein immunization is obtained by administration of at least one priming dose followed by at least one booster dose.

32. The method of claim 31, wherein the at least one priming dose comprises a first priming dose followed by a second priming dose about two or three months to about twelve months from the first priming dose and wherein the at least one booster dose is given at two years intervals.

33. The method of claim 31, wherein the immunization comprises administration of a further vaccine different from the mutated flavivirus.

34. The method of claim 33, wherein the further vaccine comprises a vector expressing a vaccine antigen, wherein the vector is derived from a virus selected from the group consisting of flavivirus, herpesvirus, poxvirus, hepadnavirus, togavirus, coronavirus, hepatitis D virus, orthomyxovirus, paramyxovirus, rhabdovirus, bunyavirus, measles, canine distemper virus and filovirus.

35. The method of claim 31, wherein the further vaccine is selected from the group consisting of a protein subunit vaccine, a toxoid vaccine, a conjugate vaccine, a DNA vaccine, a virus-like particle vaccine, a live attenuated and an inactivated vectored vaccine.

36. The method of claim 20, wherein vaccination and/or immunization is for preventing a disease, wherein the disease is selected from the group consisting of dengue fever (DF), dengue hemorrhagic fever (DHF), dengue shock syndrome (DSS), dengue fever (DF) together with dengue shock syndrome (DSS), and dengue hemorrhagic fever (DHF) together with dengue shock syndrome (DSS).

37. The method of claim 20, wherein administration is selected from the group consisting of buccal, sublingual, rectal, topical, nasal, intramuscular, intradermal and subcutaneous.

38. The method of claim 20, wherein the vaccine is to be administered at a dose of between about 1×10² to 1×10⁶ pfu.

39. The method of claim 38, wherein the dose is about 1×10² pfu.

40. The method of claim 1, comprising at least two, at least three, at least four, at least five, at least six, at least seven, at least eight or more mutated flaviviruses.

41. A method of eliciting an immune response comprising administration of a mutated flavivirus comprising at least two mutations in a nucleic acid sequence encoding for a non-structural protein 5 of the flavivirus sequence, wherein the at least two mutations lead to the inactivation of 2′O-methyltransferase activity of the non-structural protein 5, and wherein the mutated flavivirus is a dengue virus.

42. A method of vaccination, comprising administration of at least one vaccine, which is a mutated flavivirus comprising at least two mutations in a nucleic acid sequence encoding for a non-structural protein 5 of the flavivirus sequence, wherein the at least two mutations lead to the inactivation of 2′O-methyltransferase activity of the non-structural protein 5, and wherein the mutated flavivirus is a dengue virus.
